SEQUENCE LISTING (2 sequences)

SEQ ID NO: 1 - DNA, 20 bp, Homo sapiens
  source 1..20: mol_type = genomic DNA
aagtgaccagctacagtcgg

SEQ ID NO: 2 - DNA, 20 bp, Homo sapiens
  source 1..20: mol_type = genomic DNA
agcaggtctgttgcatggag

(19) United States
(12) Patent Application Publication
(10) Pub. No.: US 2024/0307532 A1
(43) Pub. Date: Sep. 19, 2024

(54) Synergistic Inhibition of eIF5A and Notch Signaling in Intermediate Tregs

(71) Applicant: The University of Toledo, Toledo, OH (US)

(72) Inventors: Shahnawaz Imam, Toledo, OH (US); Shafiya Imtiaz Rafiqi, Toledo, OH (US)

(73) Assignee: The University of Toledo, Toledo, OH (US)

(21) Appl. No.: 18/670,112

(22) Filed: May 21, 2024

Related U.S. Application Data
(63) Continuation-in-part of application No. 18/353,183, filed on Jul. 17, 2023.
(60) Provisional application No. 63/389,391, filed on Jul. 15, 2022.

(51) Int. Cl. A61K 39/395; A61K 31/155; A61K 39/00; A61P 3/10

(52) U.S. Cl. CPC A61K 39/3955; A61K 31/155; A61P 3/10; A61K 2039/505

(57) ABSTRACT

Methods for inducing plasticity in effector T cells or intermediate Treg cells to exhibit a regulatory T cell phenotype, treating an autoimmune disease, enriching Treg cells, and preparing a subject for an organ transplant are described. The methods involve the synergstic inhibition of eIF5A and Notch signaling. Also described are compositions and kits including an eIF5A inhibitor and a Notch signaling inhibitor.

### RELATED APPLICATIONS

This is a continuation-in-part application of U.S. application Ser. No. 18/353,183 filed under 35 U.S.C. § 111(a) on Jul. 17, 2023; which claims priority to U.S. Provisional Application No. 63/389,391 filed under 35 U.S.C. § 111(b) on Jul. 15, 2022. The entire disclosure of each of the aforementioned applications is incorporated herein by reference for all purposes.

### STATEMENT REGARDING FEDERALLY SPONSORED RESEARCH

This invention was made with no government support. The government has no rights in this invention.

### SEQUENCE LISTING

The sequence listing in WIPO Standard ST.26XML format named 420_63493_Seq_Listing_D2022-28.xml created on Jul. 16, 2023 and 2,682 bytes in size is incorporated herein by reference.

### BACKGROUND

Type 1 diabetes (T1D) results from the destruction of pancreatic β-cells caused by an altered immune balance in the pancreatic microenvironment. The incidence of T1D continues to rise steadily, and the ever-increasing push for more intensive management is limited by rising costs and the unremitting demand for exogenous insulin. Despite extensive research, no effective therapy has been identified to protect the β-cells from immune-mediated destruction. In T1D, unfit/low Tregs lead to increased Th1, Th17, FoxP3+IL-17+IFN-γ+ cells that culminate in cytotoxic T cells (CTLs) destroying the β-cells. Therefore, the balance between FoxP3+ expressing Treg cells (anti-inflammatory), and proinflammatory T effector cells (FoxP3+IL-17+, FoxP3+IFN-γ+, FoxP3+IL-17+IFN-γ+, IL-17+IFN-γ+, Th1, Th17 cells, and CTLs) could be the determining factor for maintaining homeostasis or promoting inflammation. Despite extensive research, there is no effective therapy to protect or restore β-cells from immune-mediated destruction. There is a need in the art for methods of treating, preventing, or ameliorating T1D.

### SUMMARY

Provided is a method for inducing plasticity in effector T cells to exhibit a regulatory T cell phenotype, the method comprising administering an effective amount of an eIF5A inhibitor to a subject so as to inhibit eIF5A in the subject; and administering an effective amount of a Notch signaling inhibitor to the subject so as to inhibit Notch signaling in the subject; wherein eIF5A and Notch signaling in the subject are inhibited simultaneously so as to induce plasticity in effector T cells in the subject to exhibit a regulatory T cell phenotype.

In certain embodiments, the eIF5A inhibitor comprises GC7. In certain embodiments, the Notch signaling inhibitor comprises an anti-DLL4 antibody.

In certain embodiments, the eIF5A inhibitor and the Notch signaling inhibitor are administered sequentially. In certain embodiments, the eIF5A inhibitor and the Notch signaling inhibitor are administered simultaneously.

In certain embodiments, the method further comprises administering a treatment for type 1 diabetes to the subject while eIF5A and Notch signaling are inhibited in the subject. In particular embodiments, the eIF5A inhibitor comprises GC7 and the Notch signaling inhibitor comprises an anti-DLL4 antibody. In particular embodiments, the treatment comprises CAR-Tregs. In particular embodiments, the CAR-Tregs are GAD65-specific CAR-Tregs. In particular embodiments where the treatment comprises CAR-Tregs, the eIF5A inhibitor comprises GC7 and the Notch signaling inhibitor comprises an anti-DLL4 antibody. In particular embodiments where the treatment comprises GAD65-specific CAR-Tregs, the eIF5A inhibitor comprises GC7 and the Notch signaling inhibitor comprises an anti-DLL4 antibody.

Further provided is a pharmaceutical composition comprising an eIF5A inhibitor, a Notch signaling inhibitor, and a pharmaceutically acceptable carrier, diluent, or adjuvant. In certain embodiments, the eIF5A inhibitor comprises GC7, and the Notch signaling inhibitor comprises an anti-DLL4 antibody.

Further provided is a kit comprising a first container housing an eIF5A inhibitor; and a second container housing a Notch signaling inhibitor. In certain embodiments, the eIF5A inhibitor comprises GC7, and the Notch signaling inhibitor comprises an anti-DLL4 antibody. In certain embodiments, the kit further comprises a treatment for type 1 diabetes (T1D). In certain embodiments, the kit further comprises GAD65-specific CAR-Tregs.

Further provided is a method of treating an autoimmune disease, the method comprising inhibiting eIF5A in a subject having an autoimmune disease; simultaneously inhibiting Notch signaling in the subject; and subsequently, administering a treatment for the autoimmune disease to the subject; wherein the simultaneous inhibition of eIF5A and Notch signaling in the subject enriches Treg cells in the subject so as to prime the subject's immune system for the treatment. In certain embodiments, eIF5A is inhibited with GC7, and Notch signaling is inhibited with an anti-DLL4 antibody. In certain embodiments, the autoimmune disease is type 1 diabetes (T1D). In certain embodiments, the treatment comprises GAD65-specific CAR-Tregs.

Further provided is a method for enriching Treg cells in a subject, the method comprising simultaneously inhibiting eIF5A and Notch signaling in a subject to enrich Treg cells in the subject. In certain embodiments, eIF5A is inhibited with GC7. In certain embodiments, Notch signaling is inhibited with an anti-DLL4 antibody. In certain embodiments, the subject is being prepared for an organ transplant.

Further provided is a method for preparing a subject for an organ transplant, the method comprising simultaneously inhibiting eIF5A and Notch signaling in a subject to prepare the subject for an organ transplant. In certain embodiments, eIF5A is inhibited with GC7. In certain embodiments, Notch signaling is inhibited with an anti-DLL4 antibody.

Further provided is a method for inducing plasticity in intermediate Treg cells, the method comprising contacting intermediate Treg cells with an effective amount of an eIF5A inhibitor and an effective amount of a Notch signaling inhibitor so as to induce plasticity in the intermediate Treg cells to exhibit a regulatory T cell phenotype.

In certain embodiments, the eIF5A inhibitor comprises GC7. In certain embodiments, the Notch signaling inhibitor comprises an anti-DLL4 antibody. In certain embodiments, the eIF5A inhibitor comprises GC7 and the Notch signaling inhibitor comprises an anti-DLL4 antibody.

In certain embodiments, the intermediate Treg cells are CD4+IFNg+IL17+FOXP3+ T cells. In certain embodiments, the regulatory T cell phenotype is CD4+CD25+FOXP3+.

In certain embodiments, the eIF5A inhibitor and the Notch signaling inhibitor are administered simultaneously.

Further provided is a method for inducing plasticity in intermediate Treg cells to exhibit a regulatory T cell phenotype, the method comprising administering an effective amount of an eIF5A inhibitor to a subject so as to inhibit eIF5A in the subject; and administering an effective amount of a Notch signaling inhibitor to the subject so as to inhibit Notch signaling in the subject; wherein eIF5A and Notch signaling in the subject are inhibited simultaneously so as to induce plasticity in intermediate Treg cells in the subject to exhibit a regulatory T cell phenotype.

In certain embodiments, the eIF5A inhibitor comprises GC7. In certain embodiments, the IF5A inhibitor and the Notch signaling inhibitor are administered simultaneously. In certain embodiments, the eIF5A inhibitor comprises GC7 and the Notch signaling inhibitor comprises an anti-DLL4 antibody.

In certain embodiments, the intermediate Treg cells are CD4+IFNg+IL17+FOXP3+ T cells. In certain embodiments, the regulatory T cell phenotype is CD4+CD25+FOXP3+.

In certain embodiments, the method further comprises administering a treatment for type 1 diabetes to the subject while eIF5A and Notch signaling are inhibited in the subject.

Further provided is a method of treating an autoimmune disease, the method comprising inhibiting eIF5A in a subject having an autoimmune disease; simultaneously inhibiting Notch signaling in the subject; and subsequently, administering a treatment for the autoimmune disease to the subject; wherein the simultaneous inhibition of eIF5A and Notch signaling in the subject enriches intermediate Treg cells in the subject so as to prime the subject's immune system for the treatment, wherein the intermediate Treg cells are CD4+IFNg+IL17+FOXP3+ T cells.

In certain embodiments, the intermediate Treg cells are enriched to CD4+CD25+FOXP3+ Treg cells.

Further provided is a method of diagnosing a subject with T1D or LADA, the method comprising obtaining a blood sample from the subject; analyzing the blood sample to determine an amount of CD4+CD25−IFNg+IL17+FOXP3+ T cells present; comparing the determined amount of CD4+CD25−IFNg+IL17+FOXP3+ T cells present in the blood sample from the subject to a control amount of CD4+CD25−IFNg+IL17+FOXP3+ T cells present in a control sample from a donor without T1D or LADA; and diagnosing the subject as having T1D or LADA if the amount of CD4+CD25−IFNg+IL17+FOXP3+ T cells present in the blood sample is greater than the control amount. In certain embodiments, the method further comprises further comprises determining the subject does not have T1D or LADA if the amount of CD4+CD25−IFNg+IL17+FOXP3+ T cells present in the blood sample is less than the control amount. In certain embodiments, the blood sample comprises peripheral blood from the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B: Glycemic effects of anti-DLL4. FIG. 1A shows weekly fasting blood glucose in anti-DLL4 and control IgG treated groups (n=4 per group). FIG. 1B shows weekly body weight in anti-DLL4 and control IgG treated groups (n=4 per group). Statistical significance was determined at P<0.05(*), and means with different superscript (#) have an approaching significant difference (P=0.06 to P<0.1) between the groups.

FIGS. 2A-2D: Anti-DLL4 significantly reduces CD8 T cells and enriches the Treg population. Representative flow-cytometry dot plots of three anatomical sites are shown. FIG. 2A shows pancreas (PN), FIG. 2B shows pancreatic lymph nodes (PLN), and FIG. 2C shows spleen (SP) of mice. Single cell suspensions were stained with fluorochrome-conjugated antibodies. First, CD3 T cells were gated from the peripheral blood mononuclear cells (PBMCs) and subsequently gated for CD4, CD8, and (CD4+CD25 +FOXP3) Treg cells from all anatomical sites (n=4 per group). Data shown in histograms for CD3, CD4, CD8, and Treg cells were found in anti-DLL4 and Control IgG treated mice (FIG. 2D). Most remarkably, at PN and PLN, CD3 and CD8 T cells were significantly reduced in the anti-DLL4 treated group while Treg enrichment was recorded in PN, PLN, and SP of the anti-DLL4 treated group.

FIGS. 3A-3B: Anti-DLL4 significantly enriches the thymic Treg population. Representative flow-cytometry dot plots of the thymus of mice single cell suspensions stained with fluorochrome-conjugated antibodies are shown in FIG. 3A. Total leucocytes were gated from single cells and subsequently gated for CD3, CD4, and Treg cells (n=4 per group). Data shown in histograms for CD3, CD4, and Treg cells were seen in anti-DLL4 and control IgG treated mice (FIG. 3B). Most remarkably, significant Treg enrichment was observed in the anti-DLL4 treated group.

FIGS. 4A-4G: Anti-DLL4 improves glucose tolerance, protects islet architecture, and ultimately improves insulin secretion. Glucose tolerance test (GTT) readings pre-treatment and post 1 month treatment of anti-DLL4 (FIG. 4A) and control IgG treated mice (FIG. 4B) (n=4 mice per group) are shown. Simultaneously, glucose stimulated plasma insulin synthesis (GSIS) was measured at 0, 2, 10, and 30 min post glucose challenge in both anti-DLL4 and control IgG treated mice (4 mice per group) (FIG. 4C). Statistical significance was determined at P<0.05 (*), means with different superscript (#) have an approaching statistical difference (P=0.06 to P<0.1) between the group. FIG. 4D shows pancreases that were isolated from mice and fixed in 10% buffered formalin. Pancreas sections (2-mm thickness) were stained with hematoxylin and cosin for morphological analysis, insulitis score-degree For histological identification, localization of lymphocytic infiltration, and for classification of islets with disrupted architecture. Anti-DLL4 treated mice rescue the pancreatic islets (FIGS. 4E, 4F) in humanized T1D mice. In totality, anti-DLL4 treatment improves the serum insulin secretion (P<0.06) as compared to control IgG treated group (FIG. 4G).

FIGS. 5A-5C: Anti-DLL4 reduces antigen-specific autoantibodies. Post sacrifice of mice, sera were subjected to autoantibody analysis. Anti-GAD65 antibodies in serum were measured in anti-DLL4 and control IgG treated groups (n=4 per group) (FIG. 5A). Anti-insulin antibodies were also measured using mouse insulin autoantibody (IAA) ELISA in anti-DLL4 and control IgG treated groups (FIG. 5B) with a positive predictive value range of 0.16-10 ng/ml (n=4 per group). Anti-IA-2 autoantibodies were also measured (n=4 per group) using human IA-2 autoantibody (IA-2Ab) ELISA in anti-DLL4 and control IgG treated groups (FIG. 5C).

FIGS. 6A-6B: In vitro expression of CD25 and FOXP3 in Tregs after single/synergistic inhibition of eIF5A and/or Notch signaling using (GC7+anti-DLL4). FIG. 6A shows representative flow-cytometry dot plots and contour plots of in vitro stimulated T cells, isolated from PLN of T1D mice. Single-cell suspensions were stained with fluorochrome-conjugated antibodies. CD3 T cells were gated for CD4 and subsequently gated for Treg cells (CD25+FOXP3). Each sample was cultured in triplicate (n=8 animals per group). In vitro stimulation with anti-DLL4, GC7, GC7 +rhGAD65, anti-DLL4 +GC7 +rhGAD65 significantly enriched Treg population (FIG. 6B) by increasing the expression of CD25 and FOXP3 on CD4 T cells.

FIGS. 7A-7E: Replicative index of T cells after single/synergistic inhibition of eIF5A and/or Notch signaling using (GC7+anti-DLL4). Peripheral blood mononuclear cells (PBMCs) isolated from T1D mice peripancreatic lymph node were treated with anti-DLL4, GC7, GC7 +rhGAD65, and anti-DLL4 +GC7 +rhGAD65. All cell groups were incubated in CellTrace (CFSE-FITC, x-axis). Each sample was co-cultured in triplicate (n=8 animal per group). Thereafter purified CD4, CD8, and CD25 were isolated using mice CD4, CD8, and Regulatory T Cell Isolation Kit (#130-104-454). Histograms show replication index as analyzed by using FLOW JO_V10 proliferation assay software (FIG. 7A). Graph summarizes data of in vitro proliferation assay from above flow data. As shown, the proliferative capacity of T cells was determined by analyzing the replication of different cell types under different culture conditions. Replicative index of CD4, CD8, CD4+IFNg+IL17 and CD4+CD25+FOXP3 cells were calculated under in vitro conditions. Replicative index of Treg cells corresponded to the in vitro enrichment of Treg in anti-DLL4, GC7, GC7+rhGAD65, and anti-DLL4+GC7+rhGAD65 treated groups (FIG. 7B). The population of CD4 T cells showing plasticity toward Treg cells was further investigated. To achieve that, the proliferative index of CD4+IFNg+IL17 positive T cells was further analyzed using CFSE (FITC labeled) to track the individual proliferative cycle. The peak in the replicative index of CD4+IFNg+IL17 positive T cells in anti-DLL4+GC7+rhGAD65 treated group was significantly higher (FIG. 7C). Co-stimulation with anti-DLL4, GC7+rhGAD65, anti-DLL4+GC7+rhGAD6 significantly reduced the CD4 count as compared to conventional stimulation with anti-(CD3+CD28) (FIG. 7D). Most interestingly, it was also observed that co-stimulation with anti-DLL4+GC7+rhGAD65 significantly reduced the proliferation index of CD8 T cells (FIG. 7E).

FIG. 8: In vivo simultaneous inhibition with GC7 (4 mg/kg, 5 days a week) and Notch using anti-DLL4 (10 mg/kg, fortnightly) for 10 weeks significantly plasticized CD4+expressing FoxP3+IL-17+IFN-γ+ cells into Treg cells.

FIG. 9: Diagram depicting the believed mechanism of Treg enrichment, eIF5A and Notch signaling inhibition favors the reversal of the pro-inflammatory milieu, enriching the Treg cells in the pancreatic microenvironment (PLN and PN), and restraining the antigen-specific CD8-mediated destruction of β-cells. These interventions tip the pro-inflammatory balance toward regulation and protect/rescue T1D mouse islet β-cells in the humanized mouse model of T1D.

FIG. 10: In vitro expression of CD25 and FOXP3 in human Tregs after co-stimulation: Representative flow-cytometry (dot plots and zebra plots) of in vitro stimulated T cells, isolated from human blood having a recent onset of latent autoimmune diabetes of adult (LADA). PBMCs were isolated using FICOL gradient method. CD3 T cells were sorted using Human Pan T cell isolation kit (Cat #130-096-535). Isolated single cells were stimulated with anti-DLL4+GC7+rhGAD6 and compared to conventional stimulation with anti-(CD3+CD28). Increased expression of CD25 and FOXP3 on CD4 T cells as well as replicative index of Treg cells corresponded to the in vitro enrichment of Treg in anti-DLL4+GC7+rhGAD65 stimulated groups. It was further noticed that in the population of CD4 T cells showing plasticity towards Treg cells as well as in the same setting, plasticized Treg cells suppress the proliferation of CD4 T cells as depicted in anti-DLL4+GC7+rhGAD65 stimulated group (9.9%) compared to anti-(CD3+CD28) stimulated group (19.7%). The frequency of proliferating cells (Dye eFluor 670 (x-axis), histograms) were analyzed by using FLOW JO proliferation assay. This figure shows that anti-DLL4+GC7 induces plasticity by increasing the expression of CD25+FOXP3 in human CD4 T cells in recent onset LADA patients.

FIG. 11: Diagram of eIF5A/Notch inhibition mediated plasticity for rescuing islets and activation of CAR-T regs.

FIG. 12: Diagram of Treg cell-based therapy.

FIG. 13: Graph showing CD4+IL-17⁺IFN-γ⁺ increased in LADA (n=3-4). Data are mean±SEM. FIG. 13 shows CD4⁺ T cells expressing IL-17⁺IFN-γ⁺ in the PBMCs of LADA patients (n=3) and non-diabetic healthy donors (n=4) ex vivo. Patients showing a recent onset of LADA, who were positive for GAD65 auto-antigen, were recruited for study under the IRB-approved protocol, 10-20 cc blood was drawn and PBMCs were isolated using the FICOL gradient method. CD4⁺ T cells expressing IL-17⁺IFN-γ⁺ were found 10 times more prevalent in PBMCs of LADA patients.

FIGS. 14A-14B: Graph showing GC7+anti-DLL4 enriches CD4+IL-17⁺IFN-γ⁺ population (n=3-4). Data are mean±SEM. Since FoxP3⁺IL-17⁺IFN-γ⁺ are intracellular cytokines, a pool of Treg deficient CD4⁺ T cells (CD4⁺CD25) was created, and the lineage of IL-17⁺IFN-γ⁺ cells was traced. Next, the origin of plasticized Tregs was sought to be defined. A Treg-deficient environment was created by sorting out Treg (CD4⁺CD25⁺) cells from the CD4⁺ T cell population from LADA and non-diabetic samples using Miltenyi Biotec cell isolation kit. The ability of GC7+anti-DLL4 to push CD4⁺ T cells expressing IL-17⁺IFN-γ⁺ to adapt a Treg phenotype was then tested. The sorted Treg deficient CD4⁺ T cell population from both groups was cultured in media supplemented with GC7 (100 μM)+anti-DLL4 (10 μg/ml)+recombinant human(rh) GAD65 (4 μg/ml) and compared with conventional stimulation by anti-CD3/CD28 dyna beads for 7 days. A significantly increased number of IL-17⁺IFN-γ⁺ T cells in LADA patients that plasticized into Treg (CD4⁺CD25⁺FoxP3⁺) cells was observed (FIG. 14A). The percentage of plasticized CD4⁺CD25⁺FoxP3⁺ cells in the GC7+anti-DLL4+rhGAD65 treated group was significantly higher (P<0.05) compared to the conventional stimulation group (FIG. 14B).

FIG. 15: Dot plot showing GC7+ anti-DLL4 plasticizes enriched CD4+IL-17⁺IFN-γ⁺ into Tregs cells showing Treg signature (CD4⁺CD25⁺FoxP3⁺).

FIG. 16: GC7+ anti-DLL4 plasticized Treg deficient CD4⁺ T cells (CD4⁺CD25) into CD4⁺CD25⁺ cells by increasing their proliferation % (n=3-4). Data are mean±SEM. To quantify the degree of plasticity and proliferation percentage under GC7+anti-DLL4 influence, a Treg-deficient environment was again created by sorting out CD4⁺CD25⁺ cells from the CD4⁺ population. The Treg deficient cells were cultured in media supplemented with GC7+anti-DLL4+rhGAD65 and compared with conventional stimulation by anti-CD3/CD28 dyna beads and control media as absolute control. After 7 days of culture, the plasticized CD4⁺CD25⁺ cells compared to the total CD4⁺CD25⁻ cells (Treg deficient) were quantified using flow cytometry. It was observed that 30-40% of CD4⁺CD25⁻ cells plasticized into CD4⁺CD25⁺ cells in the GC7+anti-DLL4+rhGAD65 treated group (FIG. 16). The proliferation percentage of plasticized CD4⁺CD25⁺ cells in all groups was also quantified, and a significantly increased proliferation of CD4⁺CD25⁺ cells in GC7+anti-DLL4+rhGAD65 treated group was found (FIG. 16). This experiment clearly revealed that the plasticizing cells belong to CD4⁺CD25⁻ T cell lineage and show increased proliferation under the influence of (GC7+anti-DLL4+rhGAD65).

FIG. 17: Plasticized Tregs maintained regulatory phenotype and had suppressive potential (n=3-4). Data are mean±SEM. To determine the functional phenotype of plasticized Tregs (CD4⁺CD25⁺), a suppression assay was designed with plasticized CD4⁺CD25⁺ and freshly isolated Tresp (CD4⁺CD25⁻) cells from PBMCs obtained from the peripheral blood of the same LADA patient (n=3). The suppressive capacity of plasticized cells was evaluated by co-culturing them with freshly isolated autologous Tresp cells in Treg: Tresp ratios of 0:1, 1:1, 1:2, and 1:0 (FIG. 17). A flow cytometry analysis following the suppression assay (5 days post-co-culture) revealed that the plasticized cells (CD4⁺CD25⁺) expressed a classical Treg phenotype (CD4⁺CD25⁺FoxP3⁺) and suppressed the freshly isolated autologous T effector population.

FIG. 18: GC7 and anti-DLL4 had no adverse effect on the human CD4 T cells. Apoptotic, live, and dead cells in treated and control groups. Significant increase in dead cells posts 96 hrs (grey) and 7^{th} day (yellow) owing to plasticity and suppression of T effector cells (n=7-8). Data are mean±SEM. (*, #, ** statistically significant at least p<0.05.)

FIG. 19: Increased % of plasticized Tregs suppress Teffector cell post 96 hrs (n=6-8). Data are mean±SEM.

FIG. 20: Dot plot of control (left) and GC7+anti-DLL4 treated (right) CD4⁺CD25⁻ cells. The effect of suppression can be easily seen on the lymphocyte population in treated group (15.5% live lymphocytes) whereas in control without any suppression, the lymphocyte population is 55.3% (live lymphocytes), both from the same human sample.

FIG. 21: GC7+anti-DLL4 plasticized CD4+ deficient PBMCs into CD4⁺CD25⁺ cells by increasing their proliferation % (n=3-4). Data are mean±SEM.

FIG. 22: Plasticized Tregs from CD4+ deficient PBMCs maintained regulatory phenotype and had suppressive potential (n=3-4). Data are mean±SEM.

FIG. 23: GC7 and anti-DLL4 had no adverse effect on the human CD 4 deficient PBMCs (including B cells, macrophages, dendritic cells, NK cells, NKT cells, and CD8 cells). Apoptotic, live, and dead cells in treated and control groups. Significant increase in dead cells posts 96 hrs (grey) and 7th day (yellow) owing to plasticity and suppression of T effector cells (n=7-8). Data are mean±SEM. (Superscript †, ††, *, **, #, ζ shows statistically significant at least p<0.05 between the treated and control group.)

FIG. 24: Increased % of plasticized Tregs from CD 4 deficient PBMCs suppress Teffector cell post 96 hrs (n=6-8). Data are mean±SEM.

FIG. 25: Dot plot of control (left) and GC7+anti-DLL4 treated (right) CD4⁺CD25⁻ cells isolated from CD4 deficient PBMCs. The effect of suppression can be easily seen on the lymphocyte population in treated group (11.6%, 9.88% live lymphocytes) whereas in the control from the same human sample, the lymphocyte population is 48.8%, 32.6% (live lymphocytes) without any suppression.

FIGS. 26A-26C: FIG. 26A shows isolation of peripheral blood mononuclear cells (PBMCs) from peripheral blood of patients with T1D/LADA (n=6)/adult healthy controls (n=7) and column separation of CD4+CD25− and CD4+CD25+ T cells to quantify CD4+ IFNg+IL17+FOXP3+ intermediate Treg cell population. FIG. 26B shows significantly elevated CD4+IFNg+IL17+FOXP3+ intermediate Treg population in T1D/LADA patients. The intermediate T cell population was increased in T1D/LADA patients and belonged to the CD4+CD25− T cell subset. FIG. 26C shows dot plots of CD4+IFNg+IL17+FOXP3+ intermediate Tregs in CD4+CD25− and CD4+CD25+ T cell subsets from T1D/LADA patients and healthy adult controls representative of significantly increased intermediate CD4+CD25−IFNg+IL17+FOXP3+ Tregs in T1D/LADA patients. The statistical significance threshold was set at P≤0.05. Data are presented as the means±standard error of means (SEM).

FIGS. 27A-27G: GAD65+GC7+anti-DLL4 treatment plasticizes CD4+CD25− T cells into CD4+CD25+ T cells. FIG. 27A shows the workflow diagram of isolation of CD4+CD25− T cells from peripheral blood mononuclear cells (PBMCs) and culture with GAD65+GC7+anti-DLL4, anti-CD3/28/137 stimulation, and culture media (RPMI+20% FCS) to investigate the conversion of CD4+CD25− T cells into CD4+CD25+ T cells and their proliferation index. FIG. 27B shows a representative contour diagram and histogram of 3 independent experiments (n=4, in triplicate) of CD25+ and CD25− T cells sorted from CD4+ T cells and their proliferation assessed by CFSE (FITC) dye after 7 days of GAD65+GC7+anti-DLL4 treatment. The CD3+CD4+ cells from the left quadrant are re-gated for Tregs based on CD25+ (PE) and FOXP3+ (APC) to confirm all the CD25+ cells (77.4%) arc CD3+CD4+CD25+FOXP3+ (77.7%). Similarly, CD25−(22.2%) cells are CD3+CD4+CD25−FOXP3+ cells (18.1%). FIG. 27C shows the percentage of CD4+CD25− T cells plasticizing into CD4+CD25+ T cells under GAD65+GC7+anti-DLL4, anti-CD3/28/137 stimulation, and culture media (RPMI+20% FCS) conditions. FIG. 27D shows the proliferation percentage of CD4+CD25+ T cells and CD4+CD25− T cells under GAD65+GC7+anti-DLL4. anti-CD3/28/137 stimulation, and culture media (RPMI+20% FCS). CD25+ T cells that plasticized under the influence of antigen-specific GAD65+GC7+anti-DLL4 treatment had better proliferation capacity. FIG. 27E shows a dot plot of CD4+CD25− T cells at different stages of plasticization. transitioning into CD4+CD25+FOXP3+ Tregs cells. Four types of CD4+ cell populations are visible in GAD65+GC7+anti-DLL4 group viz. CD25−FOXP3− (IV), CD25−FOXP3+ (III), CD25+FOXP3+ (II), and CD25+highFOXP3+high(I). CD25−FOXP3− (IV. green) and CD25−FOXP3+ T cells (III. light green) transitioning into CD25+FOXP3+ (II. orange color) and CD25+highFOXP3+high T cells (I. red color). The CD25+highFOXP3+high (I. red color) is an entirely distinct population, showing high expression of CD25 and FOXP3, that has transdifferentiated from the intermediate Tregs (CD4+CD25−IFNg+IL17+FOXP3+). FIGS. 27F and 27G show dot plots of CD4+CD25− T cells plasticizing under anti-CD3/28/137 stimulation and culture media conditions. In the anti-CD3/28/137 stimulation and media group, only two cell populations are noticeable viz. CD25+FOXP3+ and CD25−FOXP3+ (FIGS. 27F, 27G). The statistical significance threshold was set at P≤0.05, n=4, in triplicate repeated thrice). Data are presented as the means±standard error of means (SEM).

FIGS. 28A-28F: GAD65+GC7+anti-DLL4 treatment plasticizes CD4 deficient peripheral blood mononuclear cells (PBMCs) into CD4+CD25+FOXP3+ Tregs withstanding proinflammatory milieu mimicking T1D/LADA. FIG. 28A shows the workflow diagram of isolation of CD4+ T cells from PBMCs and culture of CD4 deficient PBMCs with GAD65+GC7+anti-DLL4, anti-CD3/28/137 stimulation, and culture media (RPMI+20% FCS) to investigate the conversion of CD4 deficient PBMCs into CD4+CD25+FOXP3+ cells and their proliferation index. FIG. 28B shows a representative image of 3 independent experiments (n=4, in triplicate). Contour diagram of sorted CD4+ T cells from CD4 deficient PBMCs (left quadrant). CD25+ and CD25− cells were sorted from plasticized CD4 T cells (upper right quadrant), and their proliferation was assessed by CFSE (FITC) dye (histogram. lower right quadrant). FIG. 28C shows the percentage of CD4+ T cells plasticized from CD4 deficient PBMCs cultured for 7 days under GAD65+GC7+anti-DLL4, anti-CD3/28/137 stimulation, and culture media conditions (RPMI+20% FCS). FIG. 28D shows the percentage of CD4+CD25− and CD4+CD25+ Tcells from plasticized CD4+ T cells derived from GAD65+GC7+anti-DLL4, anti-CD3/28/137 stimulation, and culture media (PMI+20% FCS). A significant number of CD4+CD25− cells are converted into CD4+CD25+ cells in the GAD65+GC7+anti-DLL4 treatment group. FIG. 28E show proliferation percentage of CD4+CD25− T and CD4+CD25+ T cells derived from GAD65+GC7+anti-DLL4, anti-CD3/28/137 stimulation, and culture media (RPMI+20% FCS) conditions. Although there is a considerable proportion of plasticized CD4+CD25+ T cells in anti-CD3/28/137 stimulation and the culture media group as well, the CD4+CD25+ T cells in the GAD65+GC7+anti-DLL4 treatment group have significantly increased proliferation index. FIG. 28F shows representative dot plots and a contour diagram of 3 independent experiments of CD4 deficient PBMCs plasticizing first into CD4+ T cells followed by further transdifferentiation into CD4+CD25+FOXP3+ Tregs cells. The statistical significance threshold was set at P≤0.05. Data are presented as the means±standard error of means (SEM).

FIGS. 29A-29C: Plasticized CD4+CD25+FOXP3+ Tregs cells exhibited a functional regulatory phenotype. FIG. 29A shows the workflow diagram of isolation of plasticized CD4+CD25+ T cells derived from Treg deficient CD4+ cells cultured under GAD65+GC7+anti-DLL4, anti-CD3/28/137 stimulation, and media (RPMI+20% FCS) conditions followed by a suppression assay against freshly isolated T responsive (CD4+CD25−) cells in different ratios from the autologous patient. FIG. 29B shows the proliferation percentage of plasticized Tregs (isolated from GAD65+GC7+anti-DLL4, anti-CD3/28/137 stimulation, culture media group) and T responsive cells in different co-culture ratios in a suppression assay (n=4, in triplicate repeated 3 times). The plasticized Tregs were column purified from GAD65+GC7+anti-DLL4, anti-CD3/28/137 stimulation, culture media conditions, incubated with CFSE, and thoroughly washed before co-culture with autologous Tresp cells. FIG. 29C shows a representative image of the contour diagram and histogram of sorted CD25+ T cells and CD25− T cells cultured under GAD65+GC7+anti-DLL4 condition and their proliferation assessed by CFSE (FITC) dye post 5-day suppression assay. Importantly. Treg phenotype was maintained in the plasticized CD4+CD25+ T cells post suppression assay. The statistical significance threshold was set at P≤0.05. Data are presented as the means±standard error of means (SEM).

FIGS. 30A-30C: Plasticized CD4+CD25+FOXP3+ Tregs cells from CD4 deficient PBMCs also exhibited a functional regulatory phenotype. FIG. 30A shows the workflow diagram of isolation of plasticized CD4+CD25+ T cells from CD4 deficient PBMCs cells cultured under GAD+GC7+anti-DLL4, anti-CD3/28/137 stimulation, and culture media (RPMI+20%FCS) conditions followed by suppression assay against freshly isolated T responsive CD4+CD25− T cells in different co-culture ratios from the autologous patient. FIG. 30B shows the proliferation percentage of Tregs (derived from GAD65+GC7+anti-DLL4, anti-CD3/28/137 stimulation, culture media group) and T responsive cells in different ratios in a suppression assay (n=4, in triplicate repeated 3 times). The plasticized Tregs were column purified from GAD65+GC7+anti-DLL4, anti-CD3/28/137 stimulation, culture media conditions, incubated with CFSE, and thoroughly washed before co-culture with autologous Tresp cells. FIG. 30C shows a representative contour diagram and histogram of sorted CD25+ T cells and CD25− T cells and their proliferation assessed by CFSE (FITC) dye post 5-day suppression assay. The plasticized Tregs exhibited a suppressive phenotype and maintained their regulatory phenotype post-suppression assay. The statistical significance threshold was set at P≤0.05. Data are presented as the means±standard error of means (SEM).

FIGS. 31A-31E: No adverse effects of immunomodulatory GAD65+GC7+anti-DLL4 treatment on CD4+ T cells. FIG. 31A shows the workflow diagram of isolation of CD4+CD25− T cells from peripheral blood mononuclear cells (PBMCs) and culture with GAD65+GC7+anti-DLL4 and culture media (control) to investigate effects of GAD65+GC7+anti-DLL4 treatment on cell viability. FIG. 31B shows sorting strategies of lymphocytes in control and GC7+anti DLL4 treated group followed by gating strategy of live, dead, and apoptotic cells. FIG. 31C shows the percentage of live, dead, and apoptotic cells in culture media (control) and GC7+anti DLL4 treatment group at 24, 48, 96 hours, and 7 days (n=3 in triplicate, repeated twice). FIG. 31D shows Treg cell percentage on the 7^{th} day to determine the phenotype of live cells after 7 days in GAD65+GC7+anti-DLL4 treated group and culture media (control). FIG. 31E shows representative dot plot of autologous CD4+CD25− T cells cultured with GAD65+GC7+anti-DLL4 and culture media (control) showing significant live lymphocyte percentage in the control group versus GAD65+GC7+anti-DLL4 treated group. An absolute reduction in the live lymphocyte population observed in the treated group shows the GAD65+GC7+anti-DLL4 treatment plasticizing the CD4+CD25− T cells into Treg phenotype and consequently suppressing the autologous parent Tresp (CD4+CD25−) cell population. The statistical significance threshold was set at P≤0.05. Data are presented as the means±standard error of means (SEM), and bars with similar symbols are statistically significant.

FIGS. 32A-32D: No adverse effects on cell viability of CD8+ and other accessory cells in CD4 deficient PBMCs with immunomodulatory GAD65+GC7+anti -DLL4 treatment were observed. FIG. 32A shows the workflow diagram on the isolation of CD4 deficient PBMCs from peripheral blood mononuclear cells (PBMCs) and culture with GAD65+GC7+anti-DLL4 and culture media (control) to investigate effects of GAD65+GC7+anti-DLL4 treatment on immune cell viability. FIG. 32B shows the percentage of live, dead, and apoptotic cells in control and GAD65+GC7+anti-DLL4 treatment group at 24, 48, 96 hours and 7 days (n=3 in triplicate, repeated twice). FIG. 32C shows the Treg cell percentage on the 7^{th} day to determine the phenotype of live cells after 7-day treatment with GAD65+GC7+anti-DLL4 and culture media (control). FIG. 32D shows a dot plot of autologous CD4 deficient PBMCs cultured with GAD65+GC7+anti-DLL4 and culture media (control) showing significantly live lymphocyte percentage in control vs. GAD65+GC7+anti-DLL4 treated group. An absolute reduction in the live lymphocyte population observed in the treated group shows the GAD65+GC7+anti-DLL4 treatment plasticizing the CD4 deficient PBMCs into CD4+ T cells followed by further transdifferentiation into Treg phenotype and consequently suppressing the autologous parent Tresp (CD4+CD25−) cell population. The statistical significance threshold was set at P≤0.05. Data are presented as the means±standard error of means (SEM), and bars with similar symbols are statistically significant.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

In accordance with the present disclosure, the role of a synergistic combination of immunomodulators in inducing plasticity in T effector (FoxP3+IL-17+, FoxP3+IFN-γ+, FoxP3+IL-17+IFN-γ+, IL-17+IFN-γ+, Th1, Th17, cells and CTLs) cells has been discovered. Two cell-signaling pathways are targeted with inhibitors: the eIF5A pathway, the inhibition of which influences T helper cell dynamics toward the noninflammatory phenotype, and the Notch signaling pathway, the inhibition of which enriches Tregs and targets autoreactive CTLs. It is shown in the examples herein that the synergistic inhibition of eIF5A and Notch signaling mediates suppression of diabetogenic T cells by inducing plasticity in CD4 T cells co-expressing IL-17 and IFNgamma (IL-17+IFNg+) toward the Treg cells phenotype. This helps, for example, to enrich the combination therapy for priming the immune system before adoptively transferring CAR-Treg cells as part of a combination treatment for autoimmune diseases such as type 1 diabetes.

In accordance with the present disclosure, simultaneously inhibiting eIF5A and Notch signaling synergistically induces plasticity in effector T cells to exhibit a Treg phenotype. It has also been found that simultaneously inhibiting eIF5A and Notch signaling induces plasticity in intermediate Treg cells present in T1D or LADA patients to exhibit a Treg phenotype. These intermediate Treg cells are CD4+IFNg+IL17+FOXP3+ Treg cells. The amount of these CD4+IFNg+IL17+FOXP3+ Treg cells in a blood sample from a subject may also be used to diagnose a subject as having T1D or LADA by comparing it to the amount of CD4+IFNg+IL17+FOXP3+ Treg cells in a control sample from a healthy donor. An increased amount of CD4+IFNg+IL17+FOXP3+ Treg cells in the blood compared to a healthy (i.e., non-T1D/LADA) control indicates that the subject has T1D or LADA.

The simultaneous inhibition of eIF5A and Notch signaling can be achieved through a variety of ways, including through non-simultaneous administration of two inhibitors or prodrugs which work to cause the inhibition of eIF5A and Notch signaling simultaneously.

eIF5A, or eukaryotic translation initiation factor 5A, is a protein in humans encoded by the EIF5A gene. eIF5A is a 17 kDA highly conserved protein expressed only in actively dividing (5%) mammalian cells (lymphocytes). eIF5A is believed to catalyze peptide bond formation and help resolve ribosomal stalls, making it an elongation factor despite the “initiation factor” name. eIF5A also regulates the protein translation processes associated with tumor proliferation. eIF5A is overexpressed in diabetes. Hypusinated eIF5A is involved in immune cell differentiation and maturation of dendritic cells (DCs). Hypusinated eIF5A is significantly overexpressed in diabetogenic CD4 T cells; inhibiting hypusinated eIF5A leads to the enrichment of Treg cells. Deoxyhypusine synthase (DHS) is known to catalyze hypusination of eIF5A. The spermidine analogue N1-guanyl-1,7-diaminoheptane (also known as GC7 or N1-carbamimidoyl-1,7-diamineoheptane) is the most potent DHS inhibitor. GC7 inhibits overexpression of eIF5A, without affecting the basal expression, and results in improved glucose tolerance, greater insulin secretion, decreased immune infiltration of islets, and delay of diabetes onset/amelioration in NOD mice, and humanized T1D mice. eIF5A inhibitors other than GC7 include, but are not limited to, anti-eIF5A neutralizing antibodies, L-mimosine, ciclopirox (also known as CPX or Batrafen), deferiprone (also known as DEF), and combinations thereof. As one non-limiting example, GC7 has the following structure:

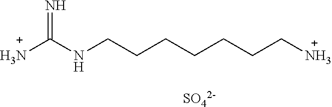

As another non-limiting example, CPX has the following structure:

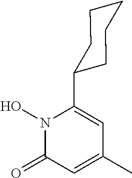

As another non-limiting example, DEF has the following structure:

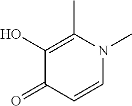

The second pathway, Notch signaling, is a cell signaling system present in most mammals. Notch signaling plays a role in many processes, such as stem cell maintenance, neuronal function and development, neurogenesis, angiogenesis, and embryonic development. The Notch signaling pathway regulates immune cell maturation and the activation and differentiation of naive CD8⁺ T cells into cytotoxic T-cells (CTLs). In T1D, β-cells are specifically killed by CTLs.

Notch signaling inhibitors include, but are not limited to, gamma-secretase inhibitors (GSIs), alpha-secretase inhibitors, delta-like protein inhibitors, jagged protein inhibitors, small molecule blockers, endosomal acidification inhibitors, blocking or negative regulatory region antibodies, stapled peptides, Notch inhibiting genes, or Notch inhibiting siRNAs, shRNAs, or microRNAs, or combinations thereof. Non-limiting examples of Notch signaling inhibitors include anti-DLL4 antibodies; anti-DLL1 antibodies; MK-0752 (also known as cis-4-[(4-chlorophenyl)sulfonyl]-4-(2,5-difluorophenyl)cyclohexanepropanoic acid); N-[N-(3,5-difluorophenylacetyl-L-alanyl)]-S-phenylglycine t-butyl ester (also known as DAPT); L685,458 (also known as (5S)-(t-butoxycarbonylamino)-6-phenyl-(4R)hydroxy-(2R)benzylhexanoyl)-L-leu-L-phe-amide); (S)-2-(2-(3,5-difluorophenyl)acetamido)-N-((S)-1-methyl-2-oxo-5-phenyl-2.3-dihydro-1H-benzo[e][1,4]diazepin-3-yl)propanamide (also known as Compound E or (s,s)-2-[2-(3,5-difluorophenyl)-acetylamino]-n-(1-methyl-2-oxo-5-phenyl-2.3-dihydro-1h-benzo[e][1,4]diazepin-3-yl)-propionamide); dibenzazepine (also known as DBZ); 7-amino-4-chloro-3-methoxyisocoumarin (also known as JLK6); [11-endo]-N-(5,6,7,8,9,10-hexahydro-6,9-methano benzo[9][8]annulen-11-yl)-thiophene-2-sulfonamide; LY 2886721 hydrochloride (also known as N-[3-[(4aS,7aS)-2-Amino-4a,5-dihydro-4H-furo[3,4-d][1.3]thiazin-7a(7H)-yl]-4-fluorophenyl]-5-fluoro-2-pyridinecarboxamide hydrochloride); and combinations thereof.

As one non-limiting example Notch signaling inhibitor, anti-DLL4 antibodies are antibodies directed against Delta-like ligand-4 (DLL4), which is one of many Notch ligands. Anti-DLL4 antibodies specifically bind to Notch receptors and inhibit Notch signaling. The anti-DLL4 antibodies may be murine or humanized antibodies. Inhibition of Notch signaling using alpha-secretase inhibitors or soluble DLL4-Fc reduces the expansion of antigen-specific CTLs. Anti-DLL4 mAbs during the induction phase of experimental autoimmune encephalomyelitis in C57BL/6 mice significantly increase the pool of CD4⁺FOXP3³⁰ Treg cells in the periphery and in the CNS. Anti-DLL4 treatment also promotes the intrathymic development of immature dendritic cells, which helps to enrich antigen-specific Treg cells and enhanced glucose-stimulated insulin secretion from islets. Subcutaneous/intraperitoneal administration of anti-DLL4@10 mg/kg body wt has been shown to have a beneficial effect in both NOD mice and the T1D mouse model used in the examples herein.

As another non-limiting example, MK-0752 has the following structure:

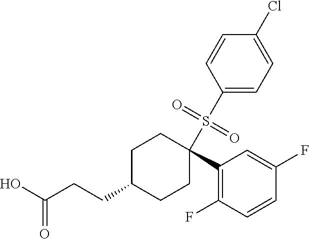 [SMILES: O=C(O)CC[C@H]1CC[C@@](c2cc(F)ccc2F)(S(=O)(=O)c2ccc(Cl)cc2)CC1]

As another non-limiting example, L685,458 has the following structure:

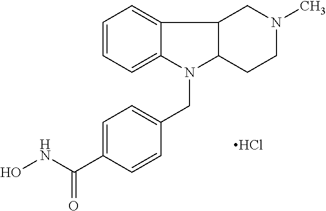 [SMILES: CN1CCC2C(C1)c1ccccc1N2Cc1ccc(C(=O)NO)cc1 ; Cl]

In some embodiments, the eIF5A inhibitor is GC7 while the Notch signaling inhibitor is anti-DLL4. Neither GC7 nor anti-DLL4 induces adverse effects in experimental models, as shown in the examples herein.

The eIF5A inhibition and Notch signaling inhibition can be achieved in many different ways, such as by administering to a subject a first agent and a second agent at the same time, where the first agent is an eIF5A inhibitor and the second agent is a Notch signaling inhibitor. Alternatively, the first agent and the second agent can be administered to the subject at different times in such a manner that causes simultaneous inhibition of eIF5A and Notch signaling. In some embodiments, the eIF5A inhibitor is GC7, and the Notch signaling inhibitor is anti-DLL4. In one non-limiting example, GC7 and anti-DLL4 are administered to the subject on the same day. In another non-limiting example, GC7 is administered to the subject every 5 days, and anti-DLL4 is administered to the subject every 14 days. However, many other combinations of inhibitors and administration schedules are possible and encompassed within the scope of the present disclosure.

The eIF5A inhibitor and the Notch signaling inhibitor can be administered together or separately. When together, the eIF5A inhibitor and the Notch signaling inhibitor can be formulated in the same pharmaceutical composition. Pharmaceutical compositions of the present disclosure may include an effective amount of an eIF5A inhibitor and an effective amount of a Notch signaling inhibitor, and/or additional agents, dissolved or dispersed in a pharmaceutically acceptable carrier. The phrases “pharmaceutical” or “pharmacologically acceptable” refer to molecular entities and compositions that produce no adverse, allergic, or other untoward reaction when administered to an animal, such as, for example, a human. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, the severity of the condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of each active ingredient (i.e., the eIF5A inhibitor and the Notch signaling inhibitor). In other embodiments, each active ingredient may comprise between about 2% to about 75% of the weight of the unit, or between about 25% to about 60%, for example, and any range derivable therein. Naturally, the amount of each active ingredient(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard-or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515; and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form should be sterile and should be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In some cases, it may be desirable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, and intraperitoneal administration. In this regard, sterile aqueous media that can be employed are known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15^{th} Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.), and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and laurocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a patch. For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation is composed of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age and weight, as well as the severity and response of the symptoms.

In particular embodiments, the compositions described herein are useful for priming the immune system in preparation for undergoing a treatment for an autoimmune disease such as T1D. Accordingly, the compositions may be used in combination therapies. That is, the compositions may be administered concurrently with or prior to one or more other desired therapeutic or medical procedures or drugs, such as a treatment for an autoimmune disease. The particular combination of therapies and procedures in the combination regimen will take into account compatibility of the therapies and/or procedures and the desired therapeutic effect to be achieved. Combination therapies include sequential, simultaneous, and separate administration of the active ingredient in a way that the therapeutic effects of the first administered procedure or drug has not entirely disappeared when the subsequent procedure or drug is administered. By way of a non-limiting example of a combination therapy, the compositions described herein can be administered in combination with one or more suitable T1D treatments such as CAR Tregs/GAD65-specific CAR Tregs. A non-limiting example CAR-Treg treatment for T1D is that described in U.S. patent application Ser. No. 17/291,853 (published as US 2022/0008522 A1) or U.S. patent application Ser. No. 17/320,663 (published as US 2022/0362294 A1), both of which are expressly incorporated herein by reference for all purposes. The combined inhibition of eIF5A and Notch signaling is also useful for priming the immune system before an organ transplant, to keep the transplanted organ alive. Therefore, another non-limiting example of a combination therapy includes the administration of a drug to prevent graft-vs-host disease, such as abatacept, in addition to the administration of an eIF5A inhibitor and a Notch signaling inhibitor.

In other embodiment, the eIF5A inhibition and Notch signaling inhibition is accomplished with separately administered agents. Thus, the compositions and methods described herein may also be made available via a kit containing one or more key components. A non-limiting example of such a kit comprises an eIF5A inhibitor and a Notch signaling inhibitor in separate containers, or a first pharmaceutical composition comprising an eIF5A inhibitor and a second pharmaceutical composition comprising a Notch signaling inhibitor in separate containers, where the containers may or may not be present in a combined configuration. Many other kits are possible, such as kits comprising a treatment for T1D, such as CAR-Tregs/GAD65-specific CAR-Tregs. The kits typically further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

T-cell receptor mediated stimulation involves low antigen expression (˜200 molecules/target cell), but studies on CAR effector T cells indicate that the density of antigen should be high on the target cell (˜2000 molecules/target cell) and there should be low expression on normal tissues in order to trigger CAR activation. Previously, insulin-specific CAR-Tregs were developed, but were unable to prevent/cure spontaneous diabetes in NOD/Ltj females, and this failure was possibly due to the low antigenic expression on islets, resulting in a failure of insulin specific CAR Tregs activation. In the pathophysiology of adult type 1 diabetes/later stages of T1D in T1D mice, islet with disrupted architecture/very few islets are viable, which may result in less activated CAR-Tregs. The islets can be rescued to produce sufficient autoantigen for activating CAR Tregs efficiently, and this can be facilitated by immunomodulation. Therefore, simultaneous inhibition of eIF5A and Notch signaling induces plasticity in CD4 T cells co-expressing IL-17 and IFN-gamma towards Treg cell phenotype, and rescues the pancreatic islet β-cells to produce endogenous autoantigen for activation of autoantigen-specific CAR-Treg cells, such as the CAR-N/M-Treg cells described in U.S. patent application Ser. No. 17/291,853 or U.S. patent application Ser. No. 17/320,663.

In the humanized mouse model of T1D, the synergistic combination of eIF5A inhibition and Notch signaling inhibition can delay the onset of T1D by a few weeks. In vivo enrichment of the Treg cells changes the dynamics, giving time for the revival of the organ. This can be an important step for subsequent CAR-Treg therapy. The combined eIF5A inhibition and Notch signaling inhibition can prime the organ for rescue.

The effects from the combined inhibition of eIF5A and Notch signaling may be transient, lasting for up to the lifetime of T cells, which is about 120 days. In the humanized T1D mouse model, rescue of the islets has been observed for up to 1 month following the combined inhibition of eIF5A and Notch signaling. The combined inhibition of eIF5A and Notch signaling described herein is useful for priming the immune system before treatment for an autoimmune disease such as T1D. The resulting inducement of T cell plasticity allows for the recovery of function in the T1D pancreas, and creates a window of opportunity for other T1D treatments. The combined inhibition of eIF5A and Notch signaling may be useful any time during the course of an autoimmune disease to temporarily ameliorate the effects of the autoimmune disease. The combined inhibition of eIF5A and Notch signaling enriches Treg cells in vivo, in some embodiments by 3-4 fold, which is beneficial for any autoimmune disease.

### EXAMPLES

### Example I

### Immune Cell Plasticity Allows for Resetting of Phenotype from Effector to Regulator with Combined Inhibition of Notch/eIF5A Pathways

Refitting of immune cells toward the non-inflammatory phenotype in the pancreas may represent a way to prevent or treat T1D. There has been developed a unique spontaneous humanized mouse model of type 1 diabetes, wherein mouse MHC-II molecules were replaced by human DQ8, and β-cells were made to express human glutamic acid decarboxylase (GAD) 65 auto-antigen. (The transgenic mice are described in U.S. application Ser. No. 16/530,452, incorporated herein by reference for all purposes.) The mice spontaneously developed T1D resembling the human disease. Humanized T1D mice showed hyperglycemic (250-300 mg/dl) symptoms by the 4th week of life. The diabetogenic T cells (CD4, CD8) present in this mouse model are GAD65 antigen-specific in nature. Intermolecular antigen spreading recorded during 3rd-6th week of age is like that observed in the human preclinical period of T1D. In these examples, whether refitting of immune cells toward the non-flammatory phenotype in the pancreas may prevent or treat T1D was tested in the humanized T1D mouse model. Two cell-signaling pathways and their inhibitions were targeted: eIF5A pathway inhibition influences T helper cell dynamics toward the noninflammatory phenotype and Notch signaling inhibition enriches Tregs, targets autoreactive CTLs, rescues the pancreatic islet structure, and increases the functionality of β-cells in terms of insulin production. The results show that inhibition of (eIF5A+Notch) signaling mediates suppression of diabetogenic T cells by inducing plasticity in CD4+ T cells co-expressing IL-17 and IFNg (IL-17⁺ IFNg⁺) toward the Treg cells phenotype.

Treg cells constitute 5-10% of the total peripheral T cells in mice as well as in humans. CD4+ Tregs have a role in maintaining immune homeostasis and preventing autoimmune reactivity. Treg cells also regulate other effector T cell functions. The majority of Treg cells are generated in the medullary region of the thymus gland as single positive CD4 T (CD4-SP) cells. Medullary thymocytes expressing higher affinity interactions with different transgene-encoded antigens are required for the development of Treg cells while lower affinity TCR do not have the ability to differentiate into Treg cells.

For proper development and function of Treg cells. Tregs are crucially dependent on the forkhead box transcription factor FOXP3; loss of Foxp3 function in humans and rodents results in devastating autoimmunity. A vast majority of Foxp3⁺ Tregs are generated during T cell development in the thymus.

Type 1 diabetes is characterized by immune-mediated destruction of pancreatic β-cells, causing lifelong dependency on exogenous insulin. Autoimmunity is an outcome of an imbalance between anti-inflammatory/pro-inflammatory immune cell ratios. These ratios decide the fate of the progression of the disease which has been well established in human T1D.

Without wishing to be bound by theory, it is believed that Treg cells in diabetic patients turn off their FOXP3 expression once they have migrated to the pancreas. This leads to a defective control of Th17 cell population, which expands and causes the destruction of pancreatic β-cells by the release of IL-17 cytokines. There is a case of a diabetic patient who had preserved fasting C-peptide levels 9 years after disease onset. The lymphocytes from the peri-pancreatic lymph node of the reported T1D patient showed IL-17 production upon GAD65 stimulation and displayed a very limited Treg suppressive ability in polyclonal assays.

Previously, the Treg/Th17 and Treg/Th1 ratios were correlated with the functionality of β-cells' insulin synthesis in a T1D mouse model. Plasticity of T helper cells has been well documented, and especially, Th17 cells acquire Th1 phenotypes. Some of the Th17 cells present in Crohn's disease are able to produce both IL-17 and IFNg, and it is believed that these proinflammatory Th17 cells may act like the Th1 type as well. Previously, the effect of eIF5A inhibition on CD4+ T cells co-expressing IL-17 and IFNg (IL-17+IFNg+) was evaluated. CD4 T cells co-expressing IL-17 and IFNg (IL-17+IFNg+) are strongly associated with plasticity of T effector toward Treg cells and have a capacity to tip the balance toward T-cell regulation. The increase in the Treg/Teffector cell (Th17 or Th1) ratio significantly increases the total pancreatic insulin content in humanized T1D mice. These findings show that there is a strong association between the Treg/Th17 and Treg/Th1 ratios and the functionality of islet β-cells in the humanized T1D mouse model; however, the increase of these ratios did not reduce cytotoxic CD8 T cells in the islets. Therefore, interventions solely targeting CD4 T cell subsets (T helper and Treg) may not be able to revert T1D, at least in the humanized T1D mouse model.

Other modulators which regulate Treg differentiation as well as ameliorate the cytotoxic T lymphocyte (CTL) function simultaneously have also been looked at Notch signaling mediates peripheral tolerance via FOXP3-dependent mechanisms as well as regulates maturation, activation, and differentiation of naive CD8 T cells into CTLs. Inhibition of Notch signaling has been done using anti-DLL4 during the induction phase of experimental autoimmune encephalomyelitis in C57BL/6 mice, by increasing the pool of regulatory T cells (Tregs) in the periphery and in the CNS. The inhibition of Notch signaling in a NOD mice model has also been done using alpha-secretase inhibitors or soluble DLL4-Fc, which reduces the expansion of antigen-specific CTLs in pancreatic β-cells. Anti-DLL4 treatment also promotes intrathymic immature dendritic cell development, which helps in the enrichment of antigen-specific Treg cells by a mechanism that requires MHCII expression on DCs and enhances glucose-stimulated insulin secretion shown to improve islet function.

Interventions using eIF5A inhibition with GC7 of CD4 T cell subsets (T helper and Treg) resulted in amelioration of T1D but was not able to revert T1D, at least in the humanized T1D mouse model, until interventions like anti-DLL4 restrained autoreactive CTLs in the islet microenvironment. Furthermore, in the examples herein, the simultaneous blockade of Notch and eIF5A signaling using anti-DLL4 and GC7 is shown, which enriches the antigen-specific Treg cell subset collectively and depletes the CD8 T cell subset in the pancreatic microenvironment.

### Materials and Methods

### Mice

C57BL/6-BTBR congenic mice carrying RIP-hGAD65-deficient murine MHC-class II molecules (mII-) were generated with the HLA-DQA1_0301/DQB1_0302 (DQ8) transgenic line that expresses HLA-DQ8 class II in the absence of endogenous murine MHC class II molecules in APCs and hGAD65 in pancreatic beta-cells. Transgenes were verified by fluorescence-activated cell sorter (FACS) and PCR. Congenic-transgenic mice were selectively in-crossed based on high fasting blood glucose for >30 generations to produce a mouse that develops diabetes spontaneously.

### Genotyping of DQ8 MHC II Haplotype by Fluorescence-Activated Cell Sorter

Peripheral blood mononuclear cells (PBMCs) were isolated from the tail vein of T1D mice, and the FACS was used for sorting a heterogeneous mixture of PBMCs. PBMC pellets were suspended in staining buffer containing anti-HLA-DQ8 (leu-10) conjugated with FITC and anti-murine MHC class II conjugated with phycoerythrin (PE). Homozygosity HLA-DQ8 was determined simultaneously for the presence of DQ8 expression and absence of mII antigens using FACS Canto (BD Biosciences) and analyzed by FLOWJO software (Tree Star Inc.).

Genotyping of RIP and hGAD65 Transgene by PCR

Genomic DNA was isolated from the tail tip of T1D mice using a ChargeSwitch™ gDNA Mini Tissue Kit (Thermo Fisher Scientific) for genotyping of homologous RIP and hGAD65 genes. The RIP-hGAD65 gene was amplified using PCR 50 primer from the 50 untranslated sequences of RIP (AAGTGACCAGCTACAGTCGG) (SEQ ID NO: 1) and a 30 primer from the coding region of the human GAD65 gene (AGCA GGTCTGTTGCATGGAG) (SEQ ID NO: 2). The amplified product (400 bp) was resolved on a 1.5% agarose gel.

### Administration of Anti-DLL4

DLL4 (delta-like 4) Armenian hamster anti-mouse, functional grade, clone: HMD4-1 (Cat#16594885, Invitrogen) and control Armenian hamster isotype control IgG (Cat#16488885, Invitrogen) were intraperitoneally administered at a dose of 10 mg/kg body wt. once a week for 2 weeks. Anti-DLL4 and control IgG-treated mice were sacrificed after 30 days of the second treatment.

### Fasting Blood Glucose, Glucose Tolerance Test (GTT), Glucose-Stimulated Insulin Secretion (GSIS), and Serum Insulin

Fasting blood glucoses were measured weekly by tail vein nicking. Mice were also subjected to the glucose tolerance test (GTT) where animals fasted for 8-10 h before being administered an intraperitoneal injection of glucose (2 g/kg body weight). Blood glucose concentrations were measured at 0, 20, 30, 60, 90, 120, 150, 180, and 210 min using the tail vein nicking technique, and blood glucose was measured with an Ascensia Breeze Glucometer (Bayer). Simultaneously, at glucose challenge, serum insulin concentrations (GSIS) were measured at 0, 2, 10, and 30 min. The insulin concentration was measured by a mouse ultrasensitive insulin ELISA kit (Crystal Chem, Inc.).

### Anti-GAD65, Anti-IA2, and Anti-Insulin Autoantibody Measurement

Anti-GAD65, anti-IA2, and anti-insulin autoantibodies were measured in mice serum using an Anti-GAD65 ELISA kit (Kronus, Star, ID) according to the manufacturer's instructions. Mice anti-insulin antibodies were measured using a mouse insulin autoantibody (IAA) ELISA kit (Abbexa, catalog#abx053161. Abbexa LLC, Houston, TX, United States; with a positive predictive value range between 0.16 and 10 ng/ml). Anti-IA2 autoantibodies were also measured using a human IA2 autoantibody (IA2Ab) ELISA kit (Kronus, Star, ID) following the manufacturers' manual.

### Procurement of Organs

After 30 days of the second dose, anti-DLL4/IgG control mice were sacrificed. Sera were saved for autoantibodies and insulin assays. Pancreases were saved for histochemistry and islet scoring. Pancreases, spleens, and peri-pancreatic lymph nodes (PLN) were isolated and processed for flow cytometric analysis.

### Immune Cell Profiling

In all flow cytometry studies, the SP, PLN, and PN cells were isolated by the mechanical method to form single cell suspensions. Cell surface staining was performed by incubating 5×10⁶ cells with fluorochrome-conjugated antibodies against mouse CD3 (clone 145-2C11, APC, APCCy7), CD4 (clone H129.19, PECy5), CD8 (clone 53-6.7, PECy7), CD25 (clone PC61, PE), or isotype controls for 20 min on ice, and were subsequently washed with buffer. A subset of T cells was permeabilized with cytofix/cytoperm fixation and permeabilization solution (BD Biosciences). Intracellular staining was performed with fluorochrome-conjugated antibodies against mouse IL-17 (clone 559502, PE), IFNg (clone 554413, APC), and forkhead box P3 (FOXP3) (clone MF23, Alexa Fluor 488, Alexa Fluor 647). Hoechst 33342 (10 mg/ml) staining was done to gate live cells containing 2n-4n cellular DNA. A BD FACSAria Ilu/FACS Canto flow cytometer (BD Biosciences) was used to acquire the cells. The data were analyzed using FLOWJO software (BD Biosciences).

### Morphological Analysis of Pancreatic Islets and Insulitis Score-Degree Classification

Pancreases were fixed in 10% buffered formalin and embedded into paraffin. Pancreas sections (2-μm thickness) were deparaffinized and stained with hematoxylin and eosin. Hematoxylin/eosin (H&E) slides were analyzed by an optical microscope for histological identification, localization of lymphocytic infiltration, and for classification of islets with disturbed architecture as previously described. Insulitis scores were determined using the grading scheme: grade 1: no islet-associated mononuclear cell infiltrates; grade 2: peri-insulitis affecting <50% of the circumference of the islet without evidence of islet invasion; grade 3: peri-insulitis affecting >50% of the circumference of the islet without evidence of islet invasion; grade 4: islet invasion. An insulitis score was obtained by dividing the total score for each pancreas by the number of islets examined. Approximately 15-20 islets/pancreas were evaluated, data were represented as mean insulitis score±SEM.

### Autoantigen Specific Proliferation of Diabetogenic T Cells

Purified CD4, CD8, and CD25 cells were isolated from T1D mice using the mice CD4 T Cell Isolation Kit (#130-104-454), CD8a+T Cell Isolation Kit II (#130-095-236), and CD4+CD25+ Regulatory T Cell Isolation Kit (Cat no: 130-091-041) following standard protocol. Briefly, after sacrificing the T1D mice, pancreatic lymph nodes (PLN) were isolated and single cell suspensions were prepared. Non-CD4 T cells and non-CD8 T cells were isolated using magnetically labeled microbeads. Non-CD4 T cells were retained in the MACS column and nontouch enriched CD4 and CD8 cells were eluted from the column. Simultaneously CD25 positive cells were separated from the CD4 clute with anti-CD25−PE microbeads with more than 90% purity. Single cell suspensions of CD4, CD8, and CD25 T cells were stained with carboxyfluorescein succinimidyl ester (CFSE) to track the induced proliferation. Single cell suspensions of purified CD4, CD8, and CD25 T cells (CFSE-labeled) were co-stimulated with recombinant human GAD65 (rGAD65) protein (4 mg/ml), GC7 (100 mM), anti-DLL4 (10 mg/ml), rGAD65+GC7, rGAD65+GC7+anti-DLL4, or CD3+CD28 stimulated for 4 days (n=7). CD4 T cells (CFSE-labeled) were further stained with fluorochrome-conjugated antibodies against mouse IL-17 (clone 559502, PE), IFNg (clone 554413, APC), and forkhead box P3 (Foxp3) (clone MF23, Alexa Fluor@488, Alexa Fluor@647). In vitro proliferation assays were analyzed by FLOWJO V10 Beta software using fix ratio, fix CV, and fix background from unstimulated cells.

### General Statistical Analysis

For glucose and insulin concentrations, anti-GAD65, anti-IA2, anti-insulin, flow cytometric data, and GTT analyses were done separately for male and female mice with a two-way ANOVA for main effects of group interactions. The significant main effects were further tested to locate the difference in means by a least significant difference test (for differences among time points in GTT for example). Data were statistically analyzed by the SAS MIXED procedure (version 9.3, SAS Institute, Inc.). The statistical significance threshold was set at P≤ 0.05. Probabilities between P>0.05 and P≤ 0.10 were regarded as approaching significance. Data are presented as the mean±SEM.

### Results

### Spontaneous Type 1 Diabetes Development in Humanized Transgenic Mice

A spontaneous humanized mouse model of T1D was generated. GAD65-specific immune cells attack and destroy the pancreatic β-cells which ultimately causes type 1 diabetes. All known stages of human T1D are recapitulated in the humanized mouse model. Moreover, the mice model develops all the classic complications of diabetes like retinopathy, nephropathy, and neuropathy. First, congenic C57BL6 and BTBR mice with compromised β-cell neogenesis/regeneration were developed. The congenic mice were made null for murine MHC-class II molecules (mII-) and were transduced with human HLA-DQ8 and GAD65 genes separately. After selective breeding of the congenic colony of mice carrying double-transgenes (DQ8-hGAD65+/+), animals with compromised β-cell function were produced. For quality control, homozygosity of DQ8 and hGAD65 was continuously monitored using FACS and PCR. Congenic mice with two human transgenes (HLADQ8 and GAD65) were subsequently crossed based on highest fasting blood glucose. After selective breeding of more than 30 generations, a founder animal was developed with spontaneous diabetes with a blood glucose of 350 mg/dl while other littermates had normal blood glucose. Spontaneous T1D mice develop diabetes spontaneously as early as the 4^{th} week of age. Most importantly, both sexes develop T1D in the spontaneous mouse model almost equally (as humans do).

### Administration of Anti-DLL4

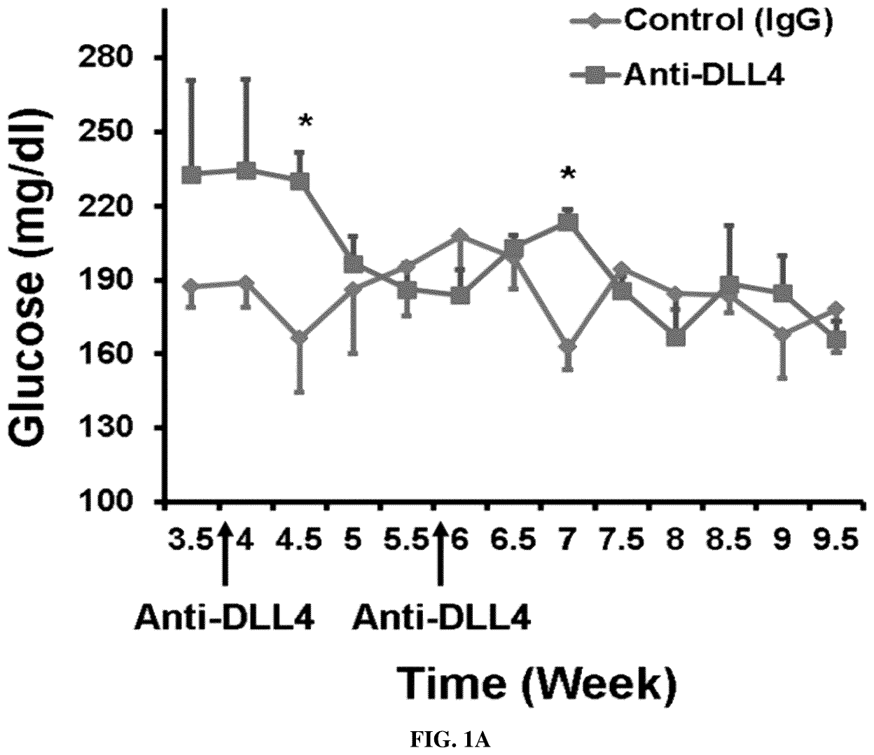

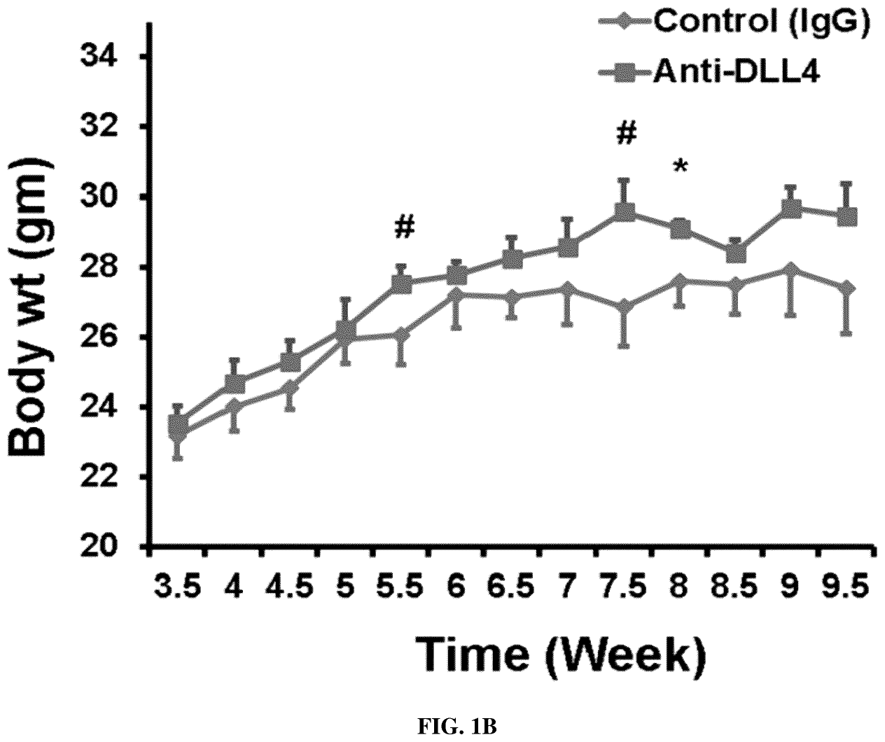

Two intra-peritoneal injections of anti-DLL4 were given at the dose rate of 10 mg/kg body weight once in 2 weeks to the T1D mouse model. Weekly blood glucose data revealed that blood glucose was reduced significantly in the anti-DLL4-treated group after the first and second treatment. Reduction in weekly glucose was maintained until the 10^{th} week with a slight fluctuation (FIG. 1A), while there were hardly any effects on body weight (FIG. 1B), although the anti-DLL4-treated group had a comparatively higher body weight.

### Administration of Anti-DLL4 Significantly Reduces CD8 T Cells and Enriches the Treg Population

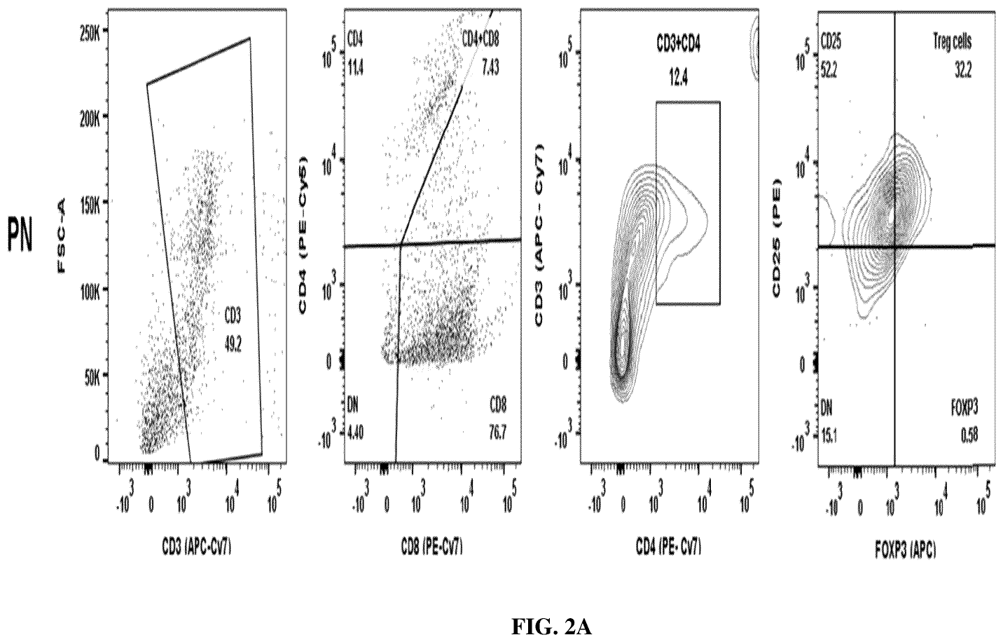

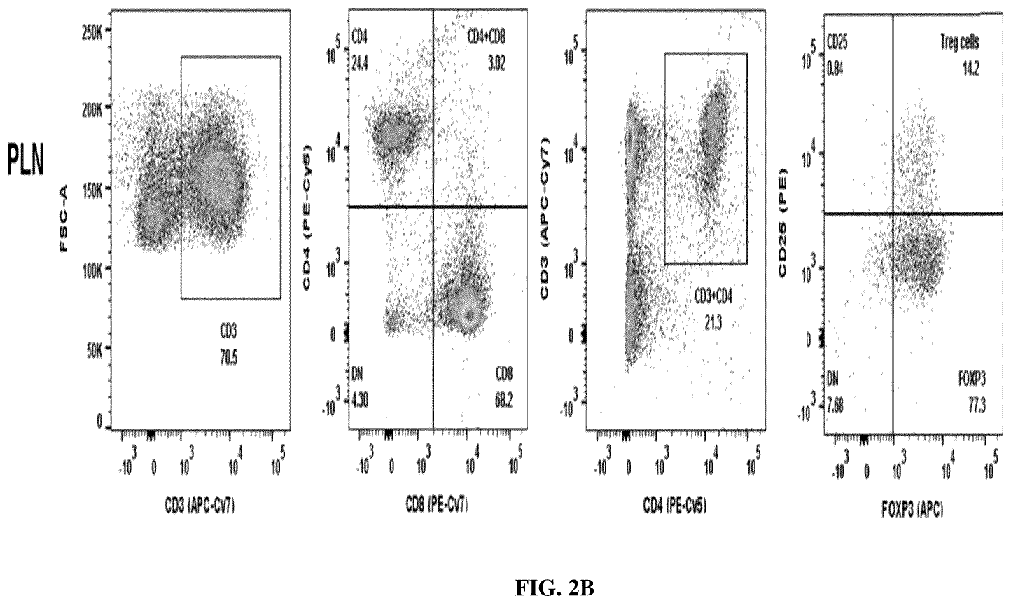

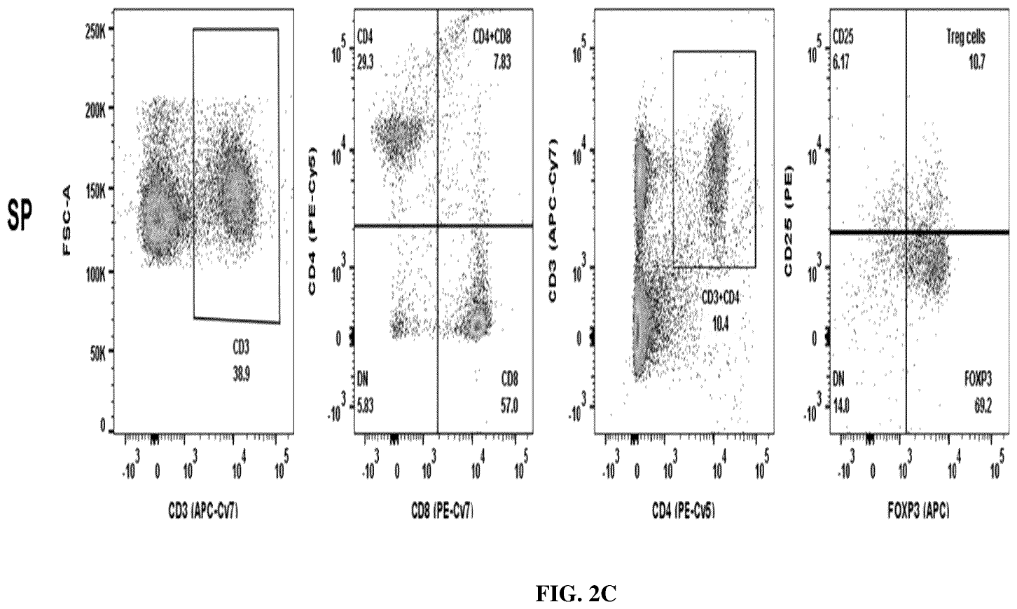

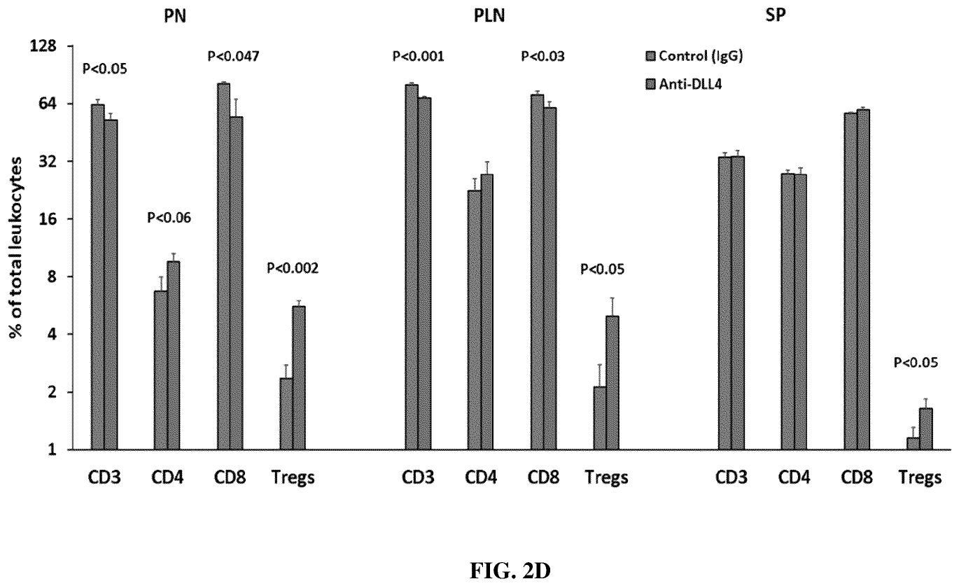

The data showed that inhibition of Notch signaling using anti-DLL4 significantly reduced the CD3 subset in the pancreatic microenvironment (PN and PLN). Reduction of CD3s was followed by reduction in CD8 T cells in the same organs (PN and PLN). The reduction in CD3s was investigated, and it was found that the reduction was actually of CD8s, which led to a reciprocal increment of CD4 Treg cells. Consecutively, inhibition of Notch signaling significantly enriched the Treg population at PN (FIG. 2A), PLN (FIG. 2B), and SP (FIG. 2C). Most interestingly, it was observed that depletion of CD8 was at the expense of enrichment of the Tregs phenotype (CD3+CD4+CD25+FOXP3+) (FIG. 2D) and (CD3+CD4+CD25+). An overall increase in CD25 expression in the CD4 T cell subsets was also observed. Administration of Anti-DLL4 Significantly Enriches the Thymic Treg Population

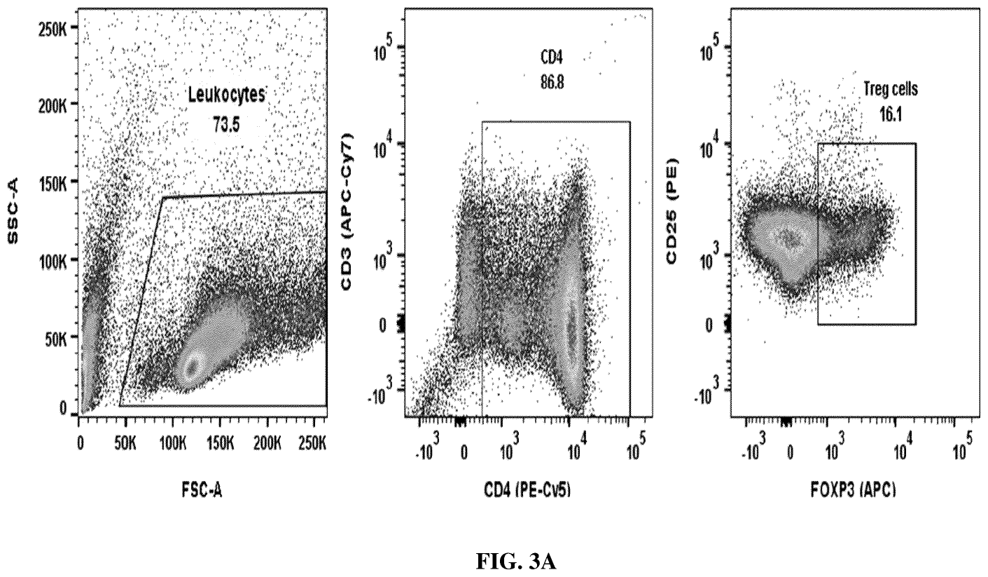

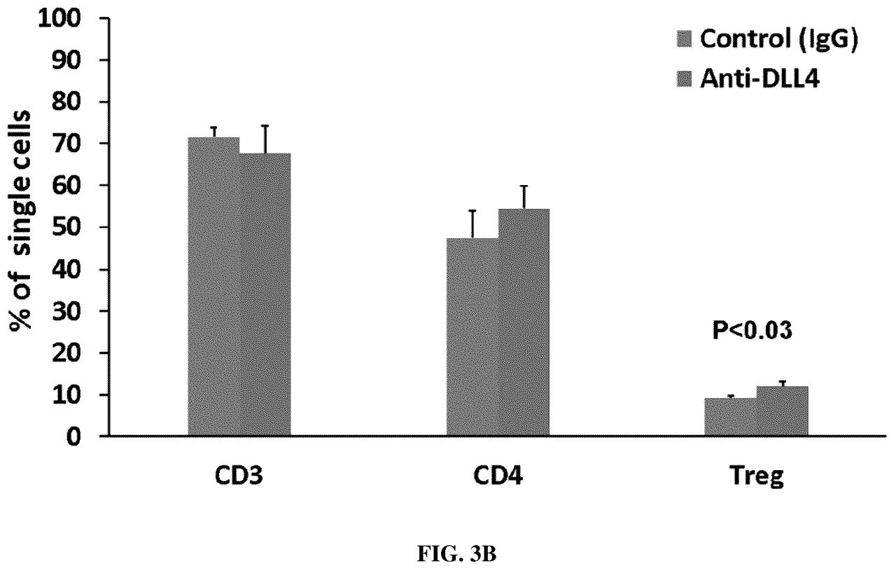

The majority of conventional Treg cells are generated in the thymus. Thymic Tregs are permanent Tregs and inhibition of Notch signaling using anti-DLL4 significantly enriched the thymic Treg populations followed by enrichment of the thymic CD4 T cell population (FIGS. 3A-3B). Although anti-DLL4 treatment enriched the CD4 positive population, it could not obtain the level of significance (P<0.22).

### Administration of Anti-DLL4 Significantly Protects the Islet Architecture and Improves Glucose Tolerance

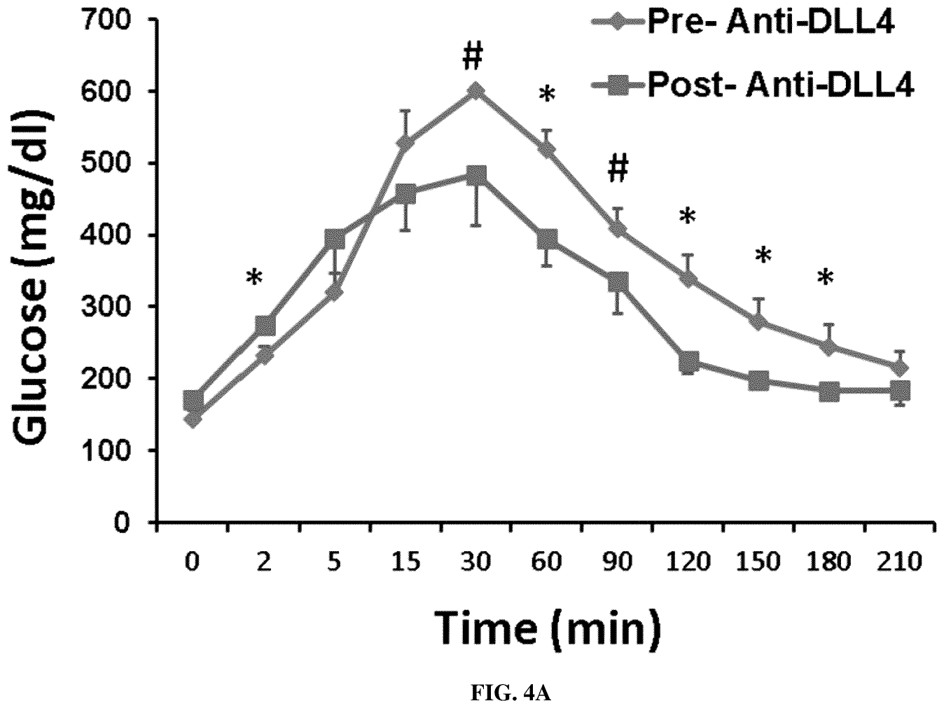

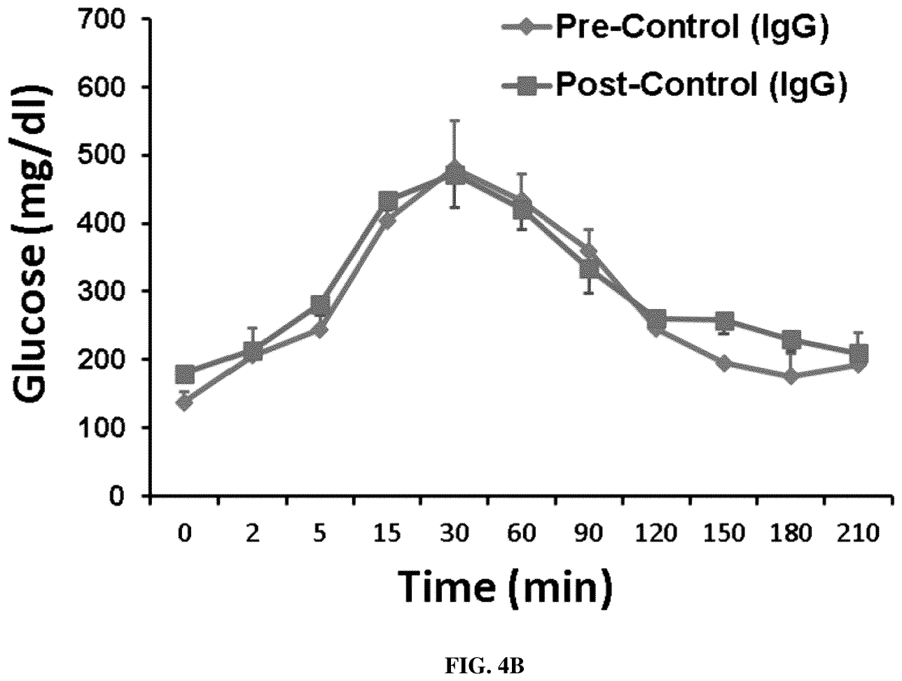

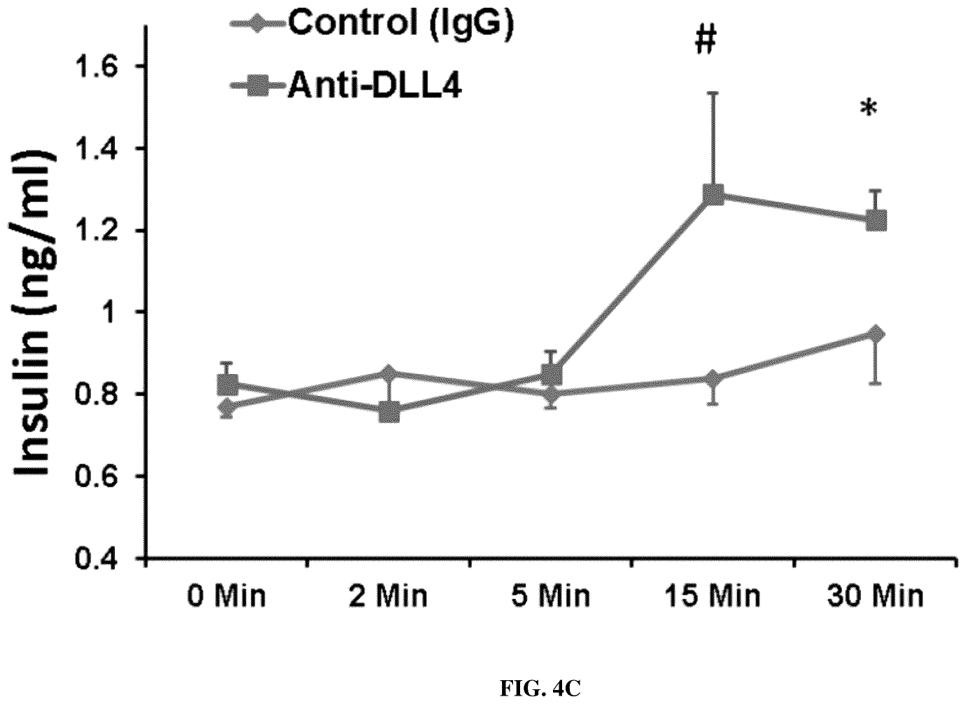

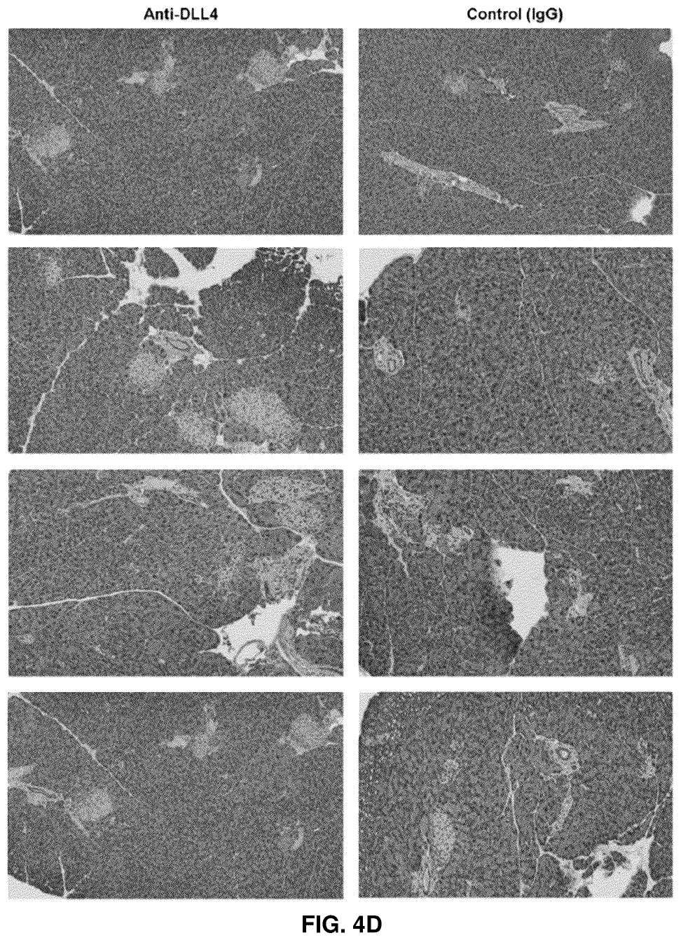

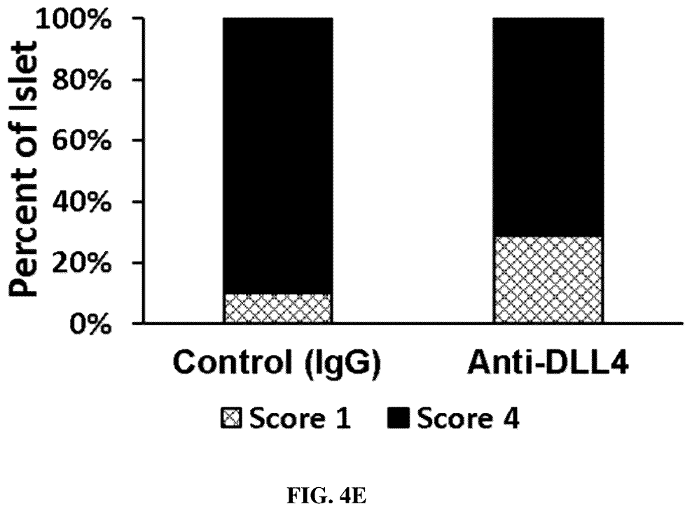

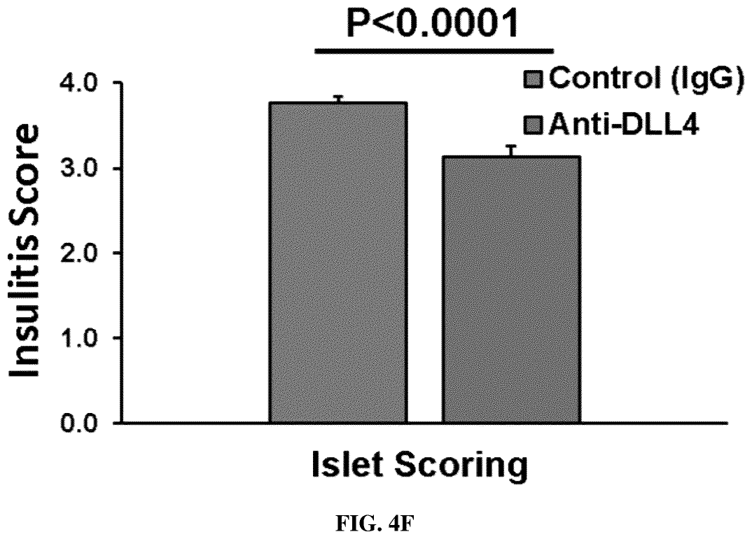

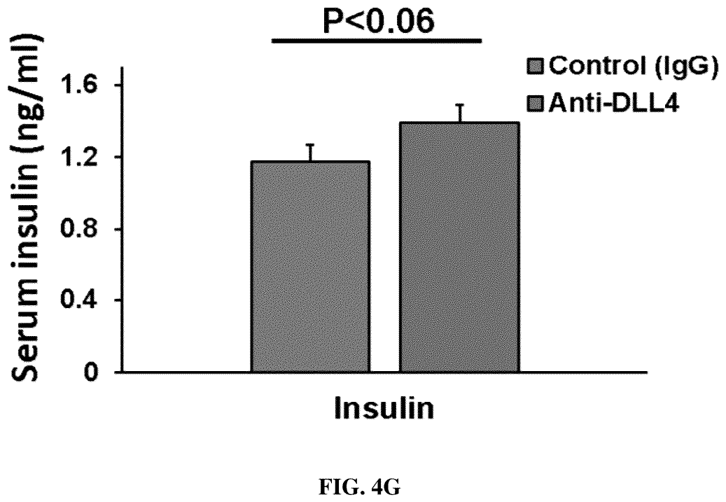

The effects of anti-DLL4 treatment on glucose tolerance (GTT) pre and post anti-DLL4 administration were compared. Intraperitoneal administration of anti-DLL4 increased the glucose tolerance at 30, 60, 90, 120, 150, and 180 min after glucose challenge (2 g/kg body wt.). The effect of anti-DLL4 treatment was significant (P≤0.05) at 60, 120, 150, and 180 min while the effect was approaching significant (P≤0.06-0.1) at 30 and 90 min as compared to pre vs. post anti-DLL4 treatment (FIG. 4A), whereas, in control (IgG) pre-and post-treatment, no significant differences were recorded (FIG. 4B). Glucose-stimulated insulin secretion (GSIS) also followed the pattern of GTT; insulin secretion increased after 5 min of glucose challenge (GTT), and increased secretion was recorded up to 30 min post glucose challenge in the anti-DLL4-treated group (FIG. 4C). The islet architecture in anti-DLL4 and control (IgG)-treated pancreases was further investigated. Anti-DLL4 treatment improved the islet architecture as well as increased the number of islets per pancreas (FIG. 4D). The islet infiltration was scored on the criteria given in the methods; anti-DLL4/control (IgG)-treated mice showed measurable insulitis. The insulitis scores were significantly reduced as compared to their control (IgG)-treated counterparts (P≥0.0001, FIGS. 4E-4F). At the end of the experiment, in total, inhibition of Notch signaling altered the pathophysiology of T1D in the humanized mouse model by improving serum insulin secretion (P<0.06) as compared to the control IgG-treated group (FIG. 4G).

### Administration of Anti-DLL4 Reduces Antigen-Specific Autoantibodies

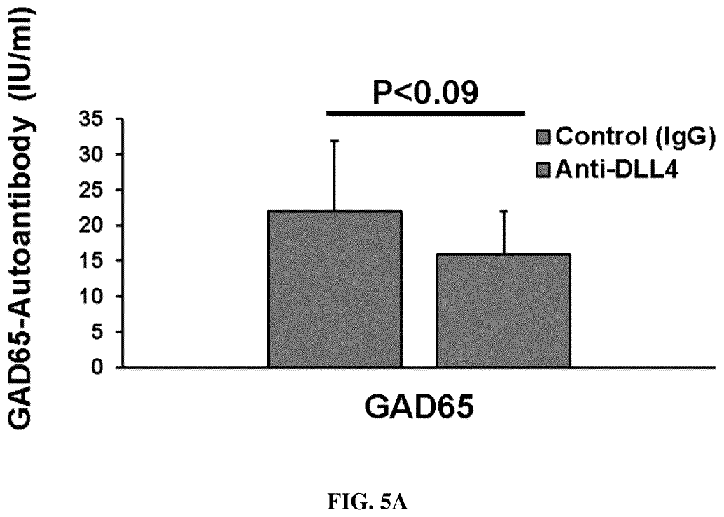

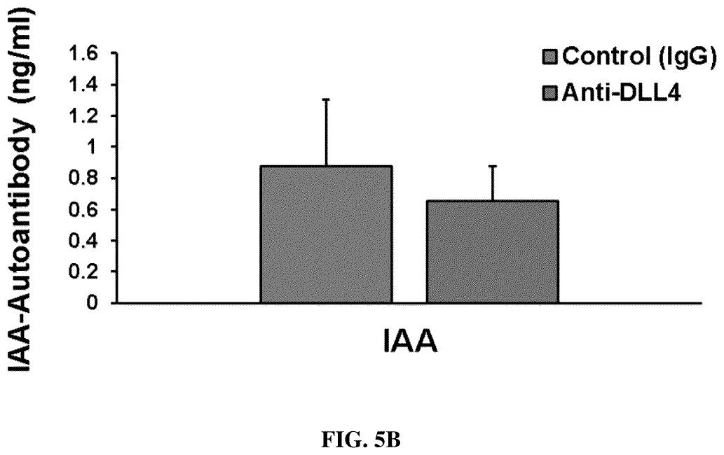

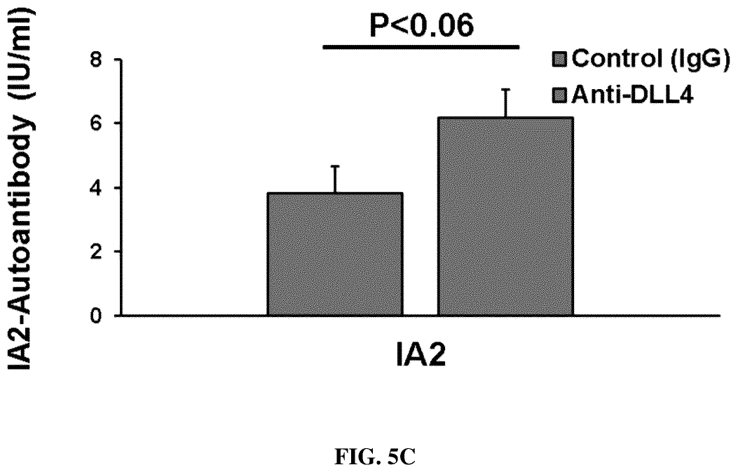

The effect of anti-DLL4 treatment on autoantibodies was further investigated by measuring the serum GAD65, IAA, and IA2 antibodies in both treated and control groups. Administration of anti-DLL4 reduced the GAD65 (P≤0.09) (FIG. 5A) and insulin autoantibodies (IAA) (FIG. 5B), while anti-DLL4 treatment increased the IA2 (P≤0.09) autoantibodies (FIG. 5C).

In vitro Expression of CD25 and FOXP3 in Tregs After Co-stimulation

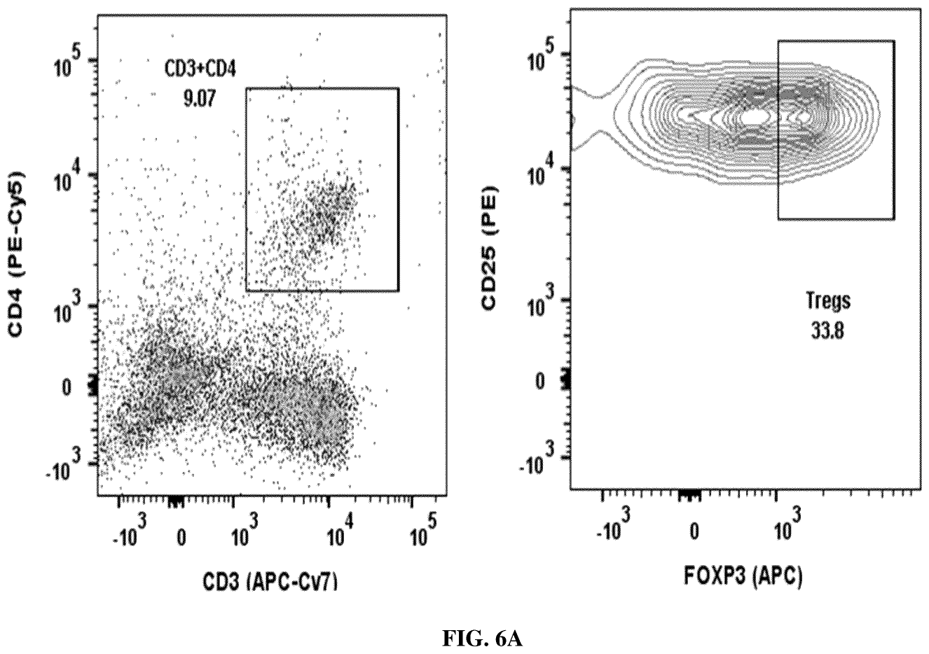

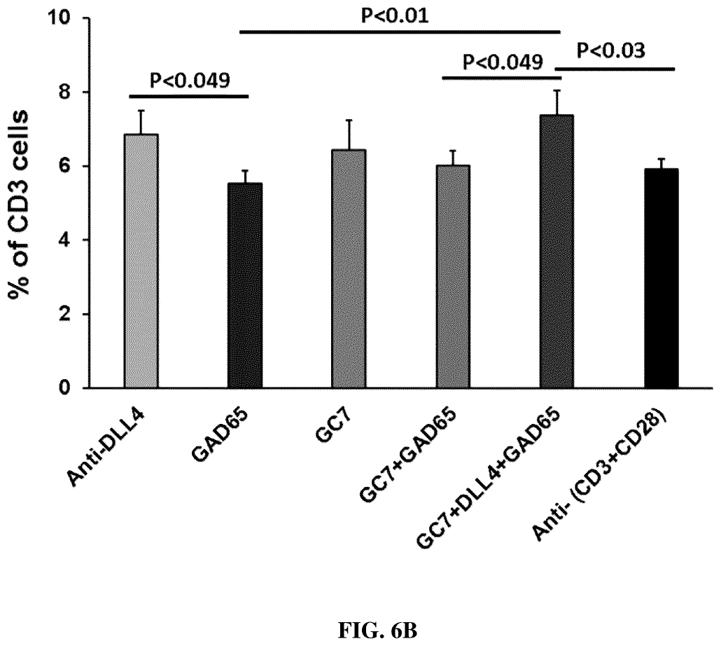

The enrichment of the Treg population was investigated upon treatment with GC7 and/or anti-DLL4, in the presence of GAD65 autoantigen in an autoantigen-specific manner or conventionally by the use of anti-(CD3+CD28). In vitro stimulation with anti-DLL4, GC7, GC7+rhGAD65, or anti-DLL4+GC7+rhGAD65 specifically and significantly enriched the Treg population (FIGS. 6A-6B) by increasing the expression of CD25 and FOXP3 (FIG. 6A) on CD3+CD4 T cells.

Replicative Index of T Cells after in Vitro Co-Stimulation

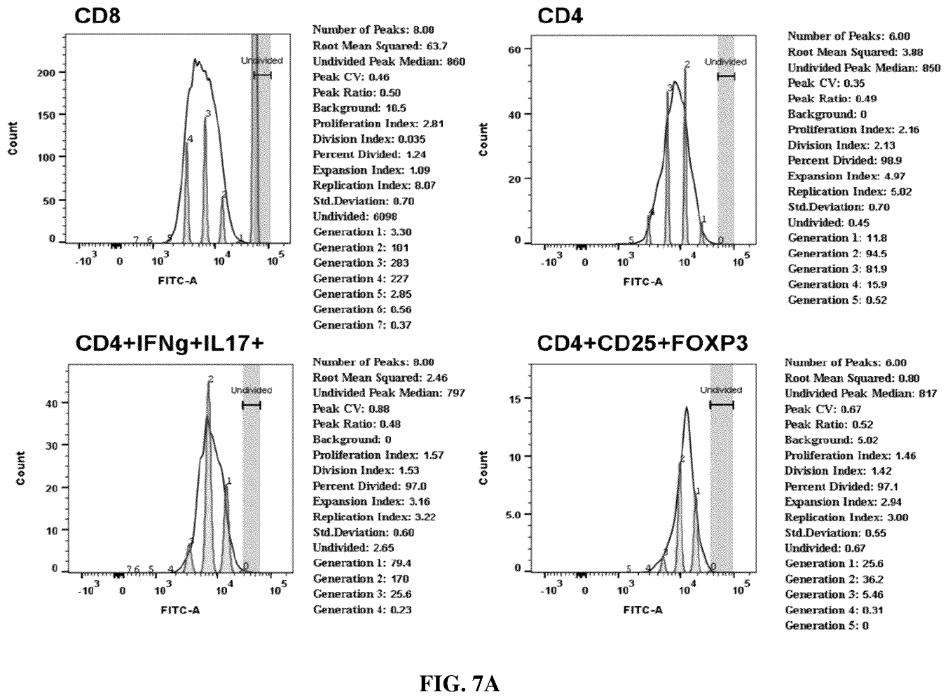

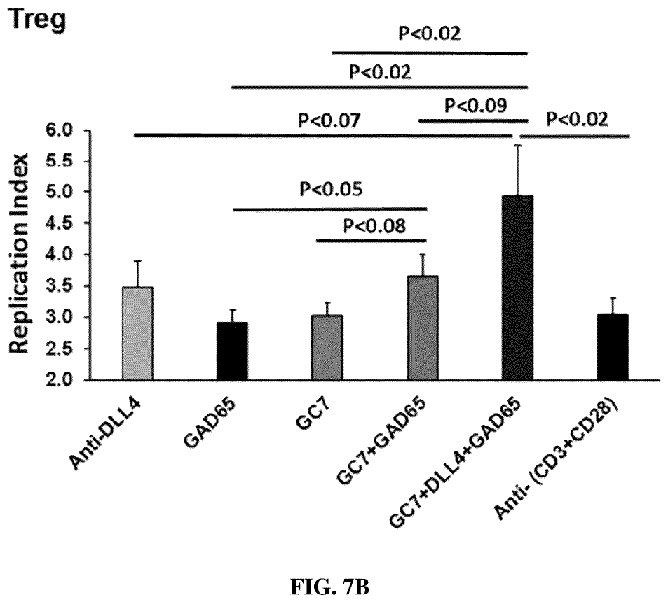

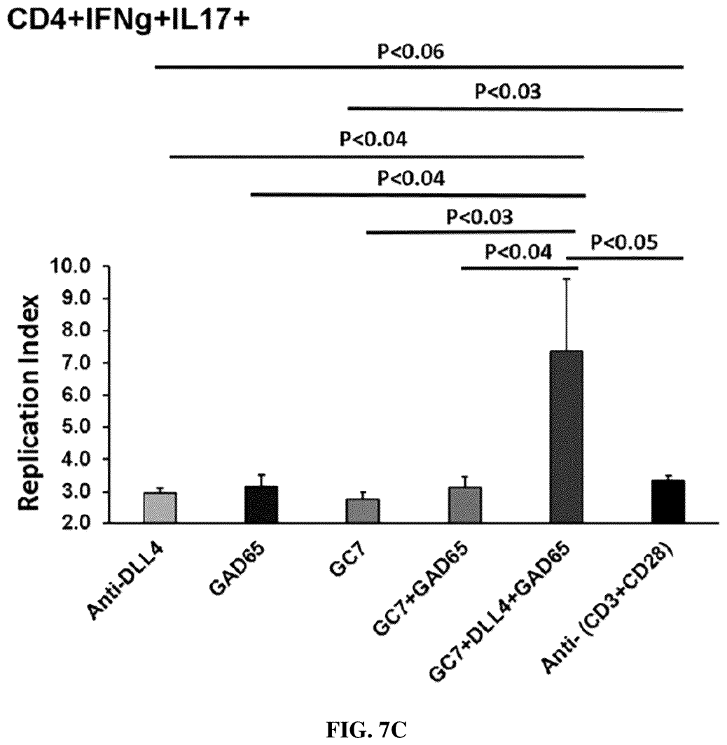

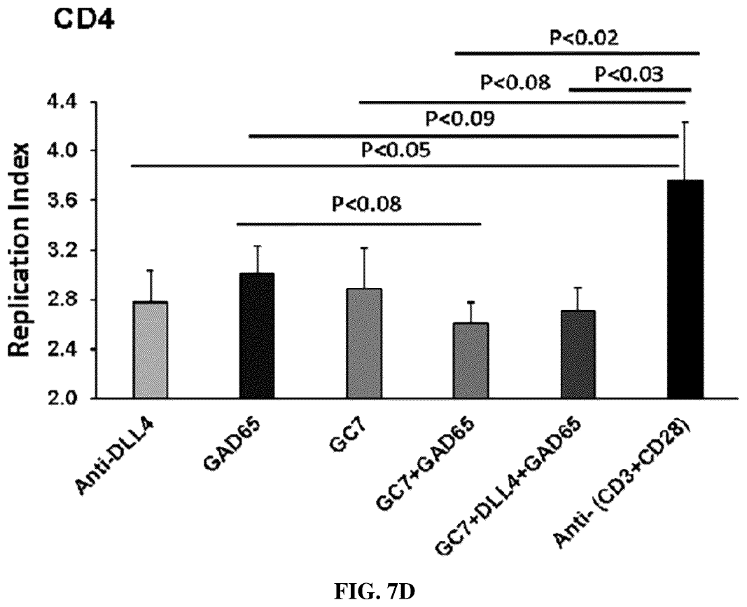

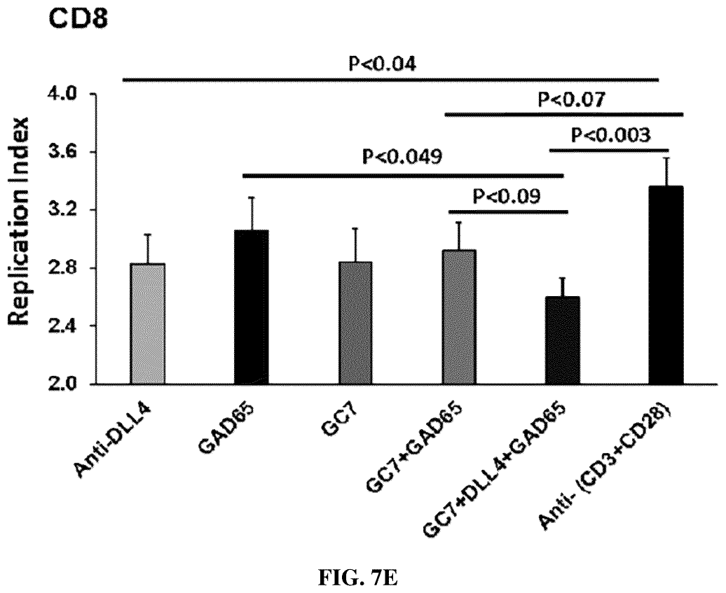

A significant Treg peak in anti-DLL4, GC7, GC7+rhGAD65, and anti-DLL4+GC7+rhGAD65-treated groups was observed. The replicative index of Treg cells under in vitro co-stimulation conditions was analyzed (FIG. 7A). Replicative index of Treg cells corresponded to the in vitro enrichment of Tregs in anti-DLL4, GC7, GC7 +rhGAD65, and anti-DLL4+GC7+rhGAD65-treated groups (FIG. 7B). Moreover, the population of CD4 T cells showing plasticity toward Treg cells was investigated. The CD4+IFNg+IL-17 positive T cells were sorted and the proliferative index was analyzed using CFSE (FITC-labeled) to track the individual proliferative cycles. The results revealed that there was a peak in the replicative index of CD4+IFNg+IL-17 positive T cells in the anti-DLL4 +GC7 +rhGAD65-treated group and was significantly higher as compared to other treated groups (FIG. 7C). Finally, the effect of co-stimulation (anti-DLL4, GC7, rhGAD65, GC7+rhGAD65, anti-DLL4+GC7+rhGAD6) on CD4 and CD8 T cells was investigated. The results show that co-stimulation with anti-DLL4, GC7+rhGAD65, and anti-DLL4+GC7+rhGAD6 significantly reduced the CD4 count as compared to conventional stimulation with anti-(CD3+CD28) (FIG. 7D). Most interestingly, co-stimulation with anti-DLL4+GC7+rhGAD6 significantly reduced the proliferation index of CD8 T cells as compared to conventional stimulation with anti-(CD3+CD28)/rhGAD65 (FIG. 7E). The reduced proliferative index of CD8 T cells was not significant in the single (anti-DLL4/GC7)-treated group. This shows that synergistic inhibition of Notch signaling and eIF5A using anti-DLL4 and GC7 can reduce (GAD65) antigen-specific CD8 T cell proliferation (FIG. 7E).

Administration of Anti-DLL4 and GC7 in T1D mice

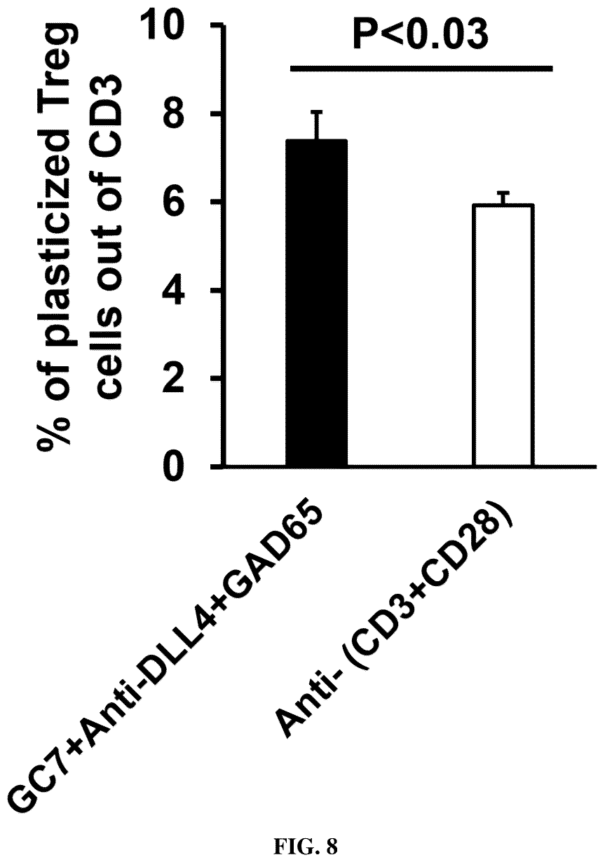

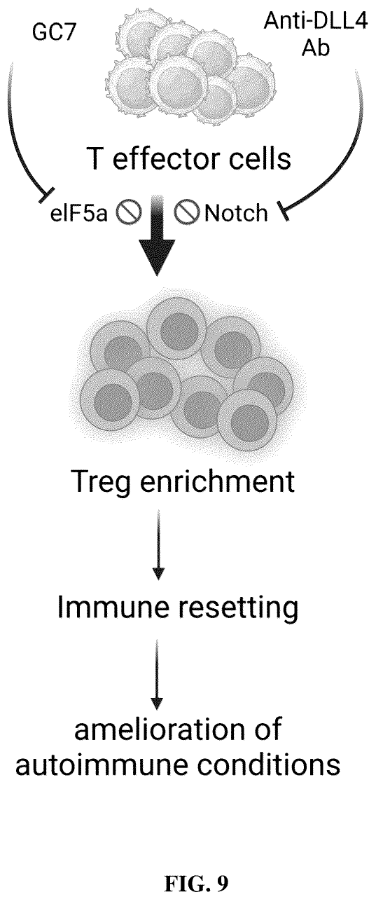

Similarly, simultaneous inhibition of eIF5A signaling using GC7 (4 mg/kg, 5 days a week) and Notch using anti-DLL4 (10 mg/kg, fortnightly) for 10 weeks intraperitoneally in T1D mice significantly plasticized CD4⁺ expressing FoxP3⁺IL-17⁺IFN-γ⁺ cells into Treg cells (FIG. 8). The Treg enrichment altered the pathophysiology of T1D by improving fasting blood glucose, glucose tolerance (GTT), and glucose-stimulated insulin secretion (GSIS) without affecting body weight. These effects were mediated by improved islet architecture as well as an increased number of islets per pancreas. Moreover, treatment with GC7 or anti-DLL4 significantly reduced the GAD65 antibody titer (humoral immune responses).

### Discussion

This example underlines the impact on antigen-specific regulation of Teffector cells and balance the composition of the Treg cell subset in the suppression of autoreactive immunity. Moreover, this example uncovers a way of switching immune cell phenotypes from effector to regulator.

Treg cells are associated with immune tolerance and constitute 5-10% of peripheral CD4 T cells in mice and humans. Tregs inhibit auto-aggressive/reactive effector T cells and simultaneously permit efficient defense against microbes preventing immune exacerbation and autoreactivity, which is known as the split effect. The split effect of Tregs implies that Treg activity is controlled in an antigen-specific manner. The specificity of Tregs is achieved by (i) formation of an antigen-specific Treg repertoire during their development in the thymus, and by (ii) the activation of the peripheral tolerance by the Treg system. In the case of autoimmunity, autoantigen reactive Treg-mediated suppression operates in an antigen-specific manner that requires engagement of TCR-antigen-MHC-II to achieve significant suppressive effect on peripheral Teffector cells. Tregs, once activated in an antigen-specific manner via their TCR, can suppress other antigen-specific Teffector cells in a bystander manner as well.

Low/unfit Treg cells in T1D patients participate in the development of T1D as compared to healthy controls, and enrichment of Treg cells is an important step to fix the Treg/Teffector imbalance for suppressing autoimmunity. Treatment with the GC7 and anti-DLL4 in spontaneous humanized T1D mice helps in amplifying antigen-specific Treg cell proliferation in the thymus, peri-pancreatic lymph nodes, pancreases, and spleen, which consecutively enrich peripheral GAD65-antigen-specific Treg cell population. Administration of GC7 and anti-DLL4 Ab in T1D mice controls hyperglycemia over time and improves the glucose tolerance test (GTT). Furthermore, it is shown herein that eIF5A and Notch inhibition can rescue pancreatic islets and confer protection to islet integrity in spontaneous humanized T1D mice and as an overall effect, increase insulin secretion.

Next, the mechanism behind the Treg enrichment post anti-DLL4 antibody treatment was investigated. Most interestingly, enrichment of CD4 and the Treg phenotype (CD3+CD4+CD25+FOXP3+) (FIG. 2D) and (CD3+CD4+CD25+) was observed. The enrichment of Treg cells was mediated through an increase in the replicative index of Treg cells (FIG. 7B). It is also shown that treatment with anti-DLL4 has a complementary effect with GC7. Synergistic inhibition of Notch and eIF5A signaling enriches Treg cells, which may be a downstream effect mediated through a two-fold increased replicative index of CD4+IFNg+IL-17+ cells (FIG. 7C) followed by an increased replicative index of Treg cells with similar intensity (FIG. 7B).

The mechanisms by which blockage of eIF5A and Notch signaling ameliorates autoimmunity are not fully understood yet. This may be mediated through impaired T helper (Th1 or Th17) immune responses or impaired/reduced antigenspecific CD4+/CD8+ T cells to the targeted organ, and/or promotion of regulatory T cell development (FIG. 2D).

Without wishing to be bound by theory, it is believed that Notch signaling upregulates the APC-mediated T helper cell responses, and engagement of Delta-like Notch ligands favors their development, whereas the data in this example revealed that blocking Notch signaling using anti-DLL4 increases the CD4 T cell count and the increment is mediated through increased CD4+CD25+FoxP3 (Treg) count. The data are in concordance with a glucose challenge in anti-DLL4-treated mice consequently leading to better second phase insulin release. The results are also in line with experiments where Notch signaling was inhibited with g-secretase inhibitors, which consecutively reduced the effects of experimental autoimmune encephalomyelitis (EAE) in a mouse model. Also, inhibition of eIF5A resets the pro-inflammatory bias in the pancreatic microenvironment by reducing Th1/Th17 cells, increase in Tregs, decrease in serum IL17 and IL21 cytokines, lowering of anti-GAD65 antibodies, and consequent ablation of the ER stress that improved functionality of the β-cells.

This example revealed that treatment with anti-DLL4 and GC7 helped enrich the peripheral and thymic Treg population which leads to preservation of islet architecture and improved the islet infiltration scoring in terms of healthy islet count per pancreas as well as serum insulin level. It also indicates improved immune tolerance. Furthermore, previously, blockage of DLL1/4 signaling has been sufficient to confer CD4+ protection against T cell-mediated rejection during allogenic bone marrow transplantation. Thus, the combined inhibition of eIF5A and Notch signaling is also useful for priming the immune system before an organ transplant, to keep the transplanted organ alive.

Notch signaling is also associated with the upregulation of the transcriptional regulator comesodermin (Eomes) which regulates the expression of perform and granzyme B in naive CD8+ T cells and helps differentiate T cells into cytotoxic T lymphocytes (CTLs). Notch 1 antisense transgene and GSI-mediated inhibition of Notch signaling attenuates CTL function by decreasing the expression of Eomes, perform, and granzyme B in mice, and reduces cytotoxic T cell activity in a transplant mouse model. Notch signaling blockage on splenic CD8+ T cells changes cytokine secretory patterns, decreases IFNg production, and increases the production of IL-10.

Taken together, this example shows that Notch signaling participates in regulating genes necessary for CTL cytotoxicity, differentiation, and function. Therefore, treatment with anti-DLL4 ameliorates the differentiation of CTLs and its cytotoxic function. A similar observation has been recorded in this example where anti-DLL4 alone or in combination with GC7 significantly reduced the CD8 T cell population in PN and PLN in vivo (FIG. 2D) as well as in vitro by reducing the replicating index of antigen-specific CD8 T cells (FIG. 7E).

Inhibition of Notch signaling prevents allograft rejection in a lung transplant mouse model by enhancing Treg survival, proliferation, and suppressive functions. It has also been demonstrated that expansion of Tregs is attributable to decreased apoptosis of peripheral Tregs as well as increased Treg proliferation. In this example, an in vitro co-stimulation experiment revealed that synergistic stimulation with GC7+anti-DLL4 enriches the antigen-specific Treg population by increasing the expression of CD25 and FOXP3 (FIG. 6B) and decreasing the proliferative index of CD8 (FIG. 7E). This is likely because of antigen-specific IL-17+IFNg+ producing T-helper cell elasticity within the T-helper subset.

Autoantigens presented by antigen-presenting cells lead to differentiation of naive CD4+ T cells into different subsets of T helper (Th) cells (Th1, Th2, Th17, and iTreg cells), and these differentiations are cytokines milieu-dependent. For example, T-bet is required for differentiation of Th1 cells, RORgt for Th17 cells, and Foxp3 for iTreg cells. Plasticity between Th1, iTreg, and Th17 cells has been reported under certain cytokine milieu conditions. iTregs can convert to IL-17-producing cells upon stimulation with IL-6 and IL-21, whereas Th17 cells may also reprogram into IFN-g-producing Th1 cells under stimulation with IL-12. Mechanisms behind the IL-17+IFNg+-producing CD4 cells' plasticity have been documented but not defined. Herein, the plasticity of IL-17+IFNg+-producing CD4 cells toward Treg as proportional to the increased proliferative efficacy of IL-17+IFNg+-producing CD4 cells is shown (FIGS. 7A-7E, 8). Therefore, antigen-specific-mediated co-stimulation with GC7+anti-DLL4 induces plasticity in T helper subsets toward Tregs as it is well established that Th1 and Th17 cells are microenvironment cytokine milieu-dependent.

The flexibility of Treg and Th17 cell differentiation provides a model system where the plasticity and unstable phenotypes of Tregs, Th1, Th17, and Th17+IFNg+cells, FoxP3+IL-17+, FoxP3+IFN-γ+, FoxP3+IL-17+IFN-≡+, IL-17+IFN-γ+ have important biological implications for designing therapeutic regimens to control autoimmunity. Simultaneous inhibition with GC7+anti-DLL4 suppresses diabetogenic T cells by inducing plasticity in CD4⁺ expressing FoxP3+IL-17+IFN-γ⁺ towards the Treg phenotype. Enriching the Treg population in the pancreatic microenvironment restrains the CTL-mediated destruction of β-cells. Since diabetogenic T cells express profuse amounts of cytokines (IFNg, IFNa, and IL-17, etc.), and cause significant oxidative stress in the pancreatic microenvironment, their suppression reduces oxidative stress by inducing anergy in diabetogenic T cells. Reduction of auto-antibody production and pancreatic microenvironment realignment helps to improve the functionality of β-cells in terms of insulin release and reduction of ER stress. Simultaneous inhibition using (GC7+anti-DLL4) leads to an improvement of β-cell mass in the late stages of diabetes when the limited numbers of islets, often with disrupted architecture, are left (FIG. 8).

### Example II

This example describes the impact of antigen-specific regulation of Teffector cells and balance composition of the Treg cell subset in the suppression of autoreactive immunity. Moreover, this example uncovers a way of switching immune cell phenotypes from effector to regulator. This example demonstrates the synergism in the use of certain immunomodulators for inducing plasticity in T effector cells toward Tregs.

As described in Example I, an in vitro co-stimulation experiment revealed that synergistic stimulation with CG7+anti-DLL4 enriches the antigen specific Treg population by increasing the expression of CD25 and FOXP3 on CD3+CD4+ T cells (FIG. 6A) and decreasing the proliferative index of CD4 and CD8 T cells (FIG. 6B).

Enrichment of Treg Cells in Human CD4 T Cells after 7 Days of GC7+Anti-DLL4+GAD65 Treatment

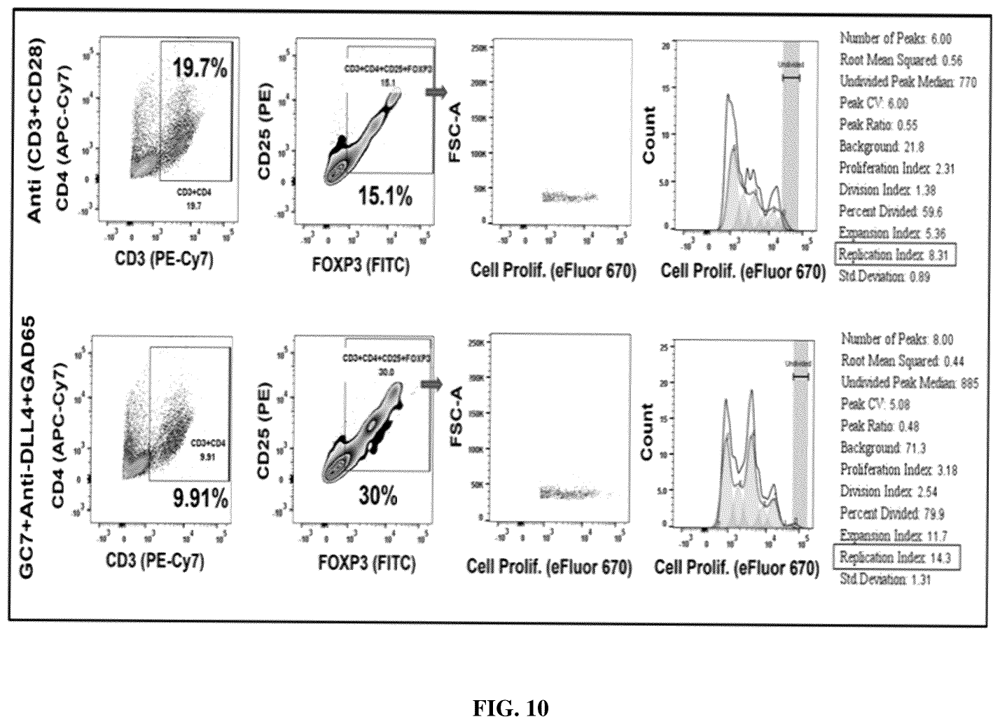

Enrichment of Treg cells with consequent delay in diabetes onset in the T1D mice indicates that CD4+IFNg+IL-17+ T cell type is primordial in disease development and progression. The investigation on human samples revealed in vitro stimulation of human CD4 T cells with anti-DLL4 (10 μg/ml)+GC7 (100 μM)+rhGAD65 (4 μg/ml) also increases the expression of CD25+FOXP3+. FIG. 10 shows representative flow-cytometry (dot plots and zebra plots) of in vitro stimulated T cells isolated from human blood having recent onset of latent autoimmune diabetes in adults (LADA) with GAD65 autoantibody titer. CD3 T cells were sorted using Human Pan T cell isolation kit. Isolated single cells were costimulated with DLL4+GC7+rhGAD6 and compared to conventional stimulation with anti-(CD3+CD28). Increased expression of CD25 and FOXP3 on CD4 T cells as well as replicative index of Treg cells corresponded to the in vitro enrichment of Treg in anti-DLL4+GC7+rhGAD65 costimulated groups. The population of CD4 T cells showed plasticity towards Treg cells; in the same setting Treg cells suppressed the proliferation of CD4 T cells in the anti-DLL4+GC7+rhGAD65 stimulated group (9.9%) compared to anti-(CD3+CD28) stimulated group (19.7). The frequency of proliferating cells (Dye eFluor 670, x-axis histograms) was analyzed by using a FLOW JO proliferation assay. In sum, in vitro studies using dual inhibition found an enriched replicative index of Tregs cells corresponding to the in vitro enrichment of Tregs in anti-DLL4+GC7+rhGAD65-treated groups.

### Use of Inducing Plasticity in Adoptive Cellular Therapy

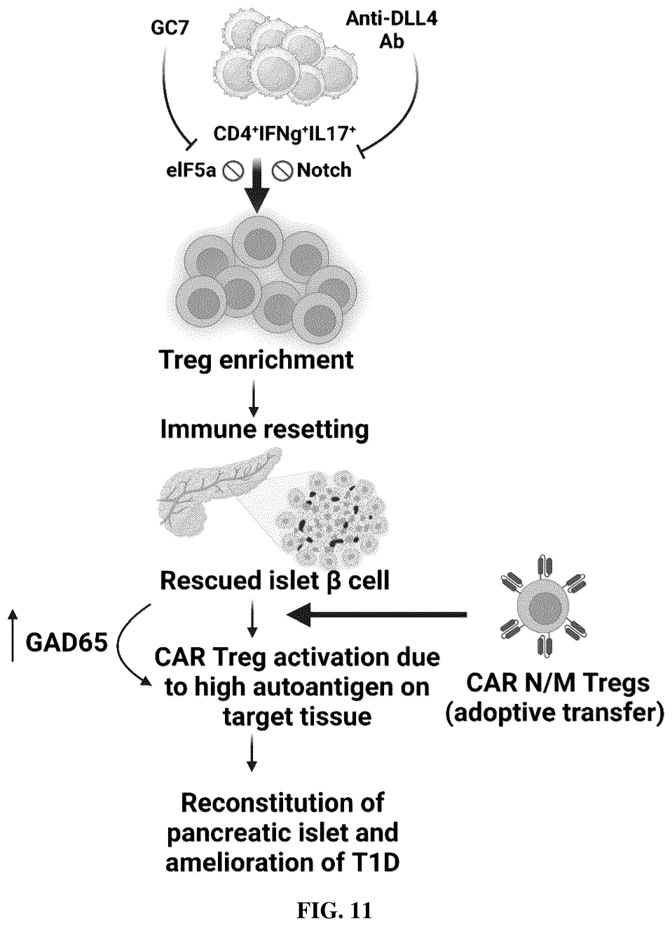

Synergistic inhibition of eIF5A and Notch using (GC7+anti-DLL4) mediates suppression of diabetogenic T cells by inducing plasticity in CD4 T cells co-expressing (IL-17+IFNg+) toward the Treg cells phenotype (refitting of Treg cells). Enrichment of Treg cells reduces the Th1 and Th17 cell bias, blocking the CTL responses. Autoreactive T cells (Th1, Th17, and CTLs) express a significant amount of cytokines (IFNg, IFNa, and IL-17, etc), inducing significant oxidative stress in the pancreatic microenvironment. Synergistic inhibition will also reduce the cytokine mediated ER stress and improve the functionality of β-cells in terms of preservation of islet architecture and insulin secretion (FIG. 11). The combination therapy based on synergistic inhibition of (Notch+eIF5A) signaling followed by adoptive transfer of GAD65 specific CAR-Tregs will first rescue the pancreatic islet cells by enriching the antigen specific Treg cells, increasing the availability of endogenous autoantigen, and ultimately activating CAR Treg cells with high potency and efficacy. This treatment approach will activate the CAR-Tregs, inducing complete anergy to activated CTLs and reconstitution of pancreatic islet. Furthermore, the eIF5A and Notch signaling need not be conducted more than once with CAR-Treg therapy, because the CAR-Tregs proliferate well once activated. This combination therapy can be immensely advantageous in adult type 1 diabetes where islets with disrupted architecture (or having very few islets being viable) can be rescued and reconstituted.

Comparison of Different Cellular Therapies in Autoimmune Diseases and Superiority of Combination Therapy by First Rescuing Organs (Islets) by Inducing Plasticity in Teffector Cells Towards Treg Cells than Adoptively Transferring the CAR-Tregs

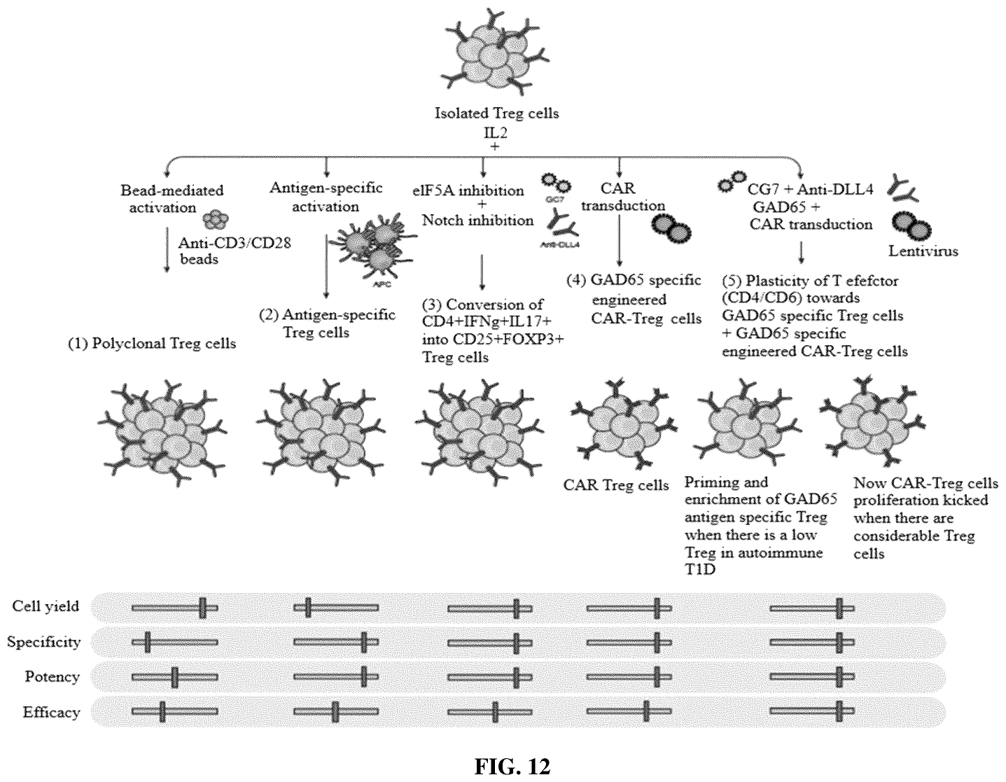

FIG. 12 depicts Treg cell-based therapy. There are five main regulatory T (Treg) cells developed for adoptive cell therapy (ACT). The first approach (1) is expanded polyclonal Treg cells that were isolated from peripheral blood/spleen and expanded in vitro using high-dose IL-2 and anti-CD3/CD28 beads to generate a high number of Treg cells. The second approach (2) is the use of antigen-presenting cells (APCs) to specifically stimulate alloreactive Treg cells from the recipient in vitro. These GAD65 antigen-specific Treg cells have been proven to be more potent than polyclonal Treg cells. However, the cell yield is relatively low after expansion and this approach owing to the low precursor frequencies of GAD65-specific-reactive Treg cells. The third approach (3) is the induced plasticity in GAD65 antigen specific Teff (CD4/CD8) cells towards GAD65 antigen-specific Treg cells using low dose immune modulator (GC7 and anti-DLL4) in the presence of GAD65-antigen. This approach involves the higher precursor frequencies of GAD65-specific autoantigen-reactive Treg cells, which may be a source for the expansion of GAD65-specific Treg cells to transduce, genetically engineered to express a synthetic receptor (chimeric antigen receptor (CAR)) that recognizes a target GAD65 autoantigen. With this approach (4), a high number of GAD65-specific CAR-Treg cells can be obtained after expansion, but this approach requires a higher number of GAD65 antigen-specific Treg cells for making an engineered CAR-Treg cells. The fifth approach (5) is the combination therapy having a synergistic effect, involving first the induction of T cell plasticity (GAD65 antigen specific Teff (CD4/CD8) cells towards GAD65 antigen specific Treg cells) after having an efficient number of GAD65-antigen-specific Treg cells. These plasticized Treg cells are used for developing engineered CAR-Treg cells with high potency and high efficacy.

The synergistic inhibition of Notch and eIF5A can be used to rescue islets by inducing plasticity in Teffector cells towards Treg cells, and thereby prime the immune system for a T1D treatment, such as with GAD65-specific CAR-Tregs.

### Example III

This example demonstrates the resetting of immune imbalance in the pancreatic microenvironment by eIF5A and Notch inhibition (immunoediting). This provides a pathway to an immunotherapy to cure, rather than simply cope with, T1D.

### Exploiting the Plasticity of CD4+ Teffector Cells

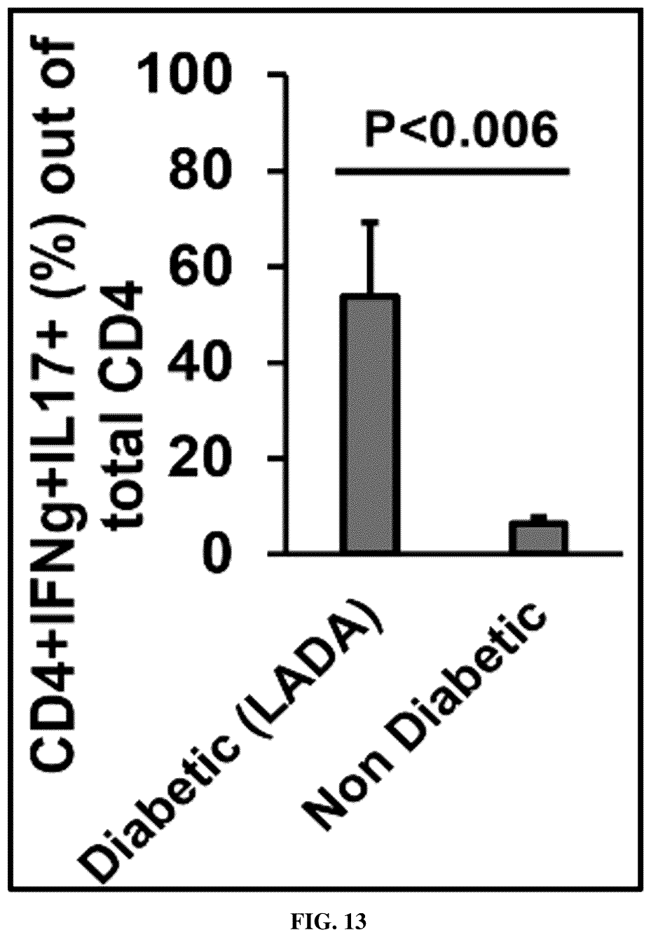

Plasticity is the ability of a single CD4+ T cell to take on characteristics of many T cell subsets simultaneously or at different times during its life cycle. Likewise in autoimmunity, T cells adopt alternative transcriptional lineages that generate functionally distinct subsets that modulate localized or specific inflammatory sites. The balance between Foxp3⁺ expressing Treg cells (anti-inflammatory), and proinflammatory cells (Foxp3+IL-17⁺, Foxp3⁺IFN-γ⁺, Foxp3⁺IL-17⁺IFN-γ⁺, effector T cells) may be the determining factor for maintaining homeostasis or promoting inflammation. An exponential increase in Foxp3⁺IL-17⁺IFN-γ⁺ T cells (an intermediate Treg subset) has been identified in diabetic (latent autoimmune diabetes in adults (LADA)) patients (n=3-4) (FIG. 13). By exploiting the transdifferentiation phenomenon, these cells can be pushed to adapt a Treg phenotype and suppress diabetogenic T cells. In the process, two key pathways that induce the differentiation of Treg from intermediate Treg subsets have been identified. Hypusinated eIF5A is involved in immune cell differentiation and maturation of dendritic cells (DCs); the outcome of inhibition of hypusinated eIF5A is the enrichment of Treg cells. The second pathway. Notch signaling, regulates immune cell maturation and the activation and differentiation of naive CD8⁺ T cells into cytotoxic T-cells (CTLs). Inhibition of Notch signaling reduces the expansion of antigen-specific CTLs and enriches the thymic Treg cells. Notch can be activated in T cells by interacting with antigen-presenting cells. However, it is believed that CD4+ T cells express notch ligands on their surfaces. It has been found that inhibiting both eIF5a and Notch signaling induces Treg differentiation. Specifically, in vitro, simultaneous inhibition of (eIF5A+Notch) signaling using (GC7+anti-DLL4) pushes the IL-17⁺IFN-γ⁺ T (proinflammatory) subset towards a Treg phenotype (anti-inflammatory) in LADA patients (FIGS. 13-15). Further, it has been shown that eIF5A and Notch inhibition significantly reduces diabetogenic Th1 cells in the pancreas (PN) and pancreatic lymph nodes (PLN), thereby transiently improving islet dysfunction and hyperglycemia of NOD and T1D mice. This exciting achievement was accomplished by plasticizing diabetogenic CD3⁺CD4⁺+IL-17⁺IFN-γ⁺ T cells into CD3⁺CD4⁺CD25⁺FOXP3⁺ Tregs. As a result, an anti-inflammatory cells/cytokine bias was created in the pancreatic microenvironment, depleting CTLs, reducing ER stress in islet β-cells, and resulting in the improvement of type 1 diabetes. This approach provides a significant advance for the field because simultaneous GC7+anti-DLL4 administration induces plasticity and enriches Treg cells. GC7+anti-DLL4 treatment induces plasticity and may be a useful therapeutic regimen for controlling autoimmunity.

Simultaneous Inhibition of eIF5A and Notch Signaling Induced Plasticity in Teffector to Tregs in Human Ex Vivo System

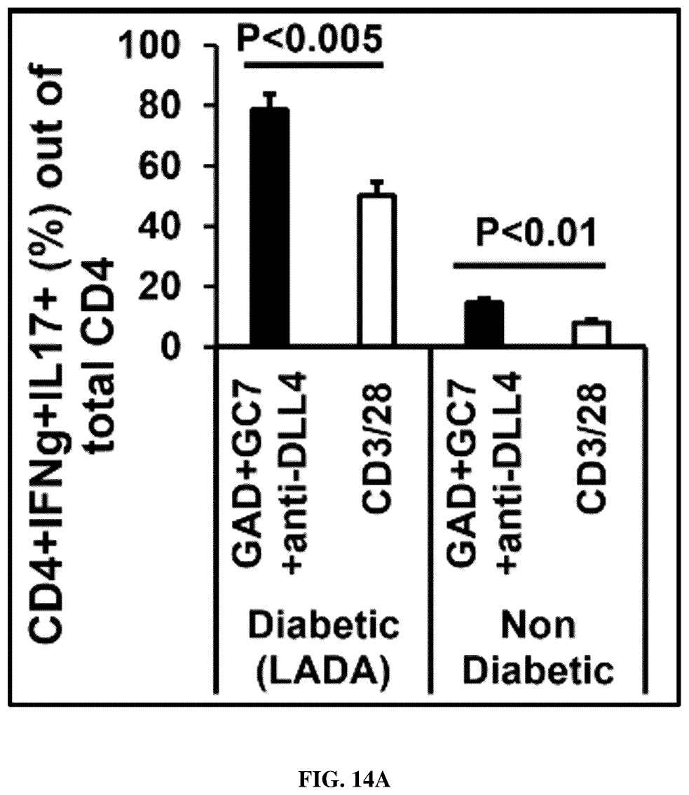

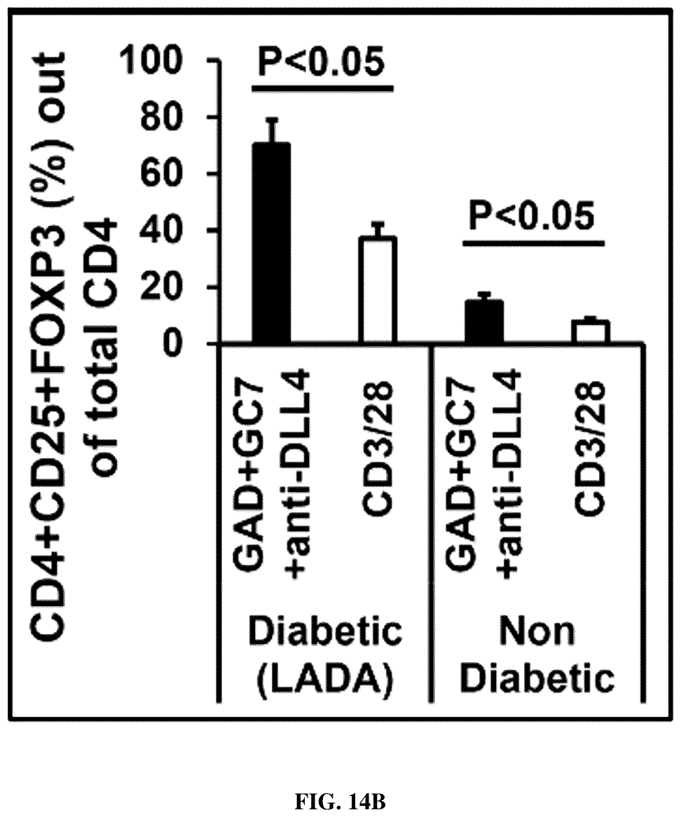

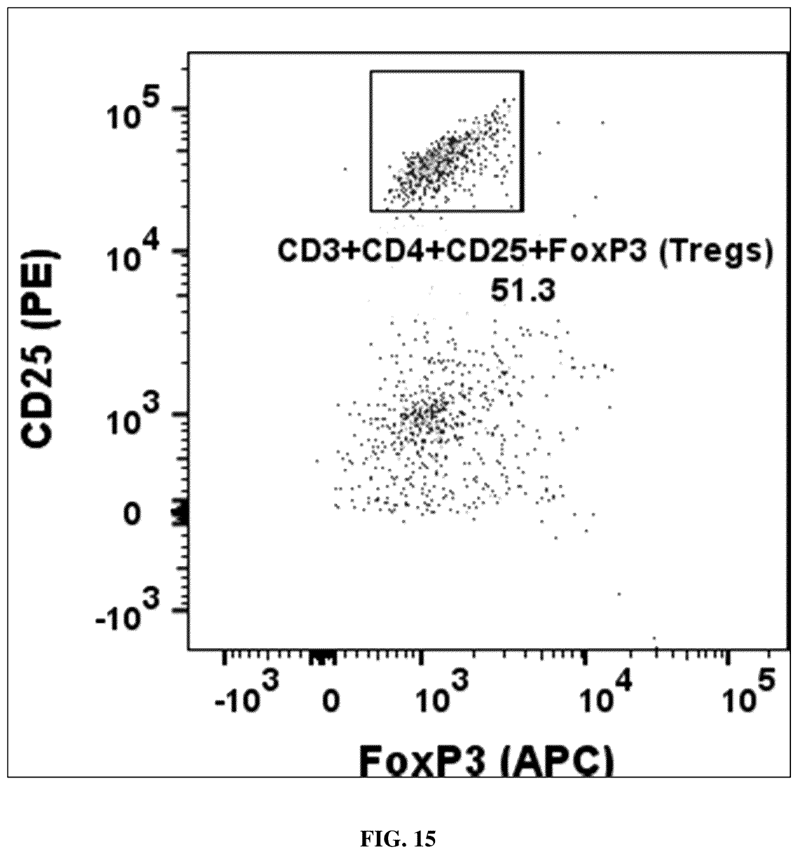

Treg stability and plasticity are regulated not only by the signals they receive during their generation but also by their microenvironment. The plasticity of CD4⁺ and CD4 deficient PBMCs (PBMCs-CD4) isolated from LADA patients (n=3) and non-diabetic healthy donors (n=4) was investigated ex vivo. Patients showing a recent onset of LADA who were positive for GAD65 auto-antigen were recruited under the IRB-approved protocol. 10-20 cc blood was drawn and PBMCs were isolated using the FICOL gradient method. The CD4 T cells expressing Foxp3⁺IL-17⁺IFN-γ⁺ in these samples were compared, and it was found that these cells were 10 times more prevalent in LADA patients (FIG. 13). Since Foxp3⁺IL-17⁺IFN-γ⁺ are intracellular cytokines, a pool of Treg deficient CD4⁺ T cells (CD4⁺CD25⁻) was created and the lineage of Foxp3⁺IL-17⁺IFN-≡⁺ cells was traced. To do so, a Treg-deficient environment was created by sorting out CD4+CD25+cells from the CD4+ population from LADA and non-diabetic samples using Miltenyi Biotec cell isolation kit. The ability of GC7+anti-DLL4 to push CD4 T cells expressing IL-17⁺IFN-γ⁺ to adapt a Treg phenotype was then tested. The sorted Treg deficient CD4⁺ population from both groups was cultured in media supplemented with GC7 (100 μM)+anti-DLL4 (10 μg/ml)+rhGAD65 (4 μg/ml) and compared with conventional stimulation by anti-CD3/CD28 dyna beads for 7 days. A significantly increased number of IL-17⁺IFN-γ⁺ T cells was observed in LADA patients that plasticized into Tregs (CD4⁺CD25⁺FoxP3⁺) (FIGS. 14A-14B). The percentage of plasticized CD4⁺CD25⁺FoxP3 cells in the GC7+anti-DLL4+rhGAD65 treated group was significantly higher (P<0.05) compared to the conventional stimulation group (FIG. 15). Next, defining the origin of plasticized Tregs was attempted.

(CD4+CD25−) T Cells are Plasticized into (CD4+CD25+) Tregs Cells

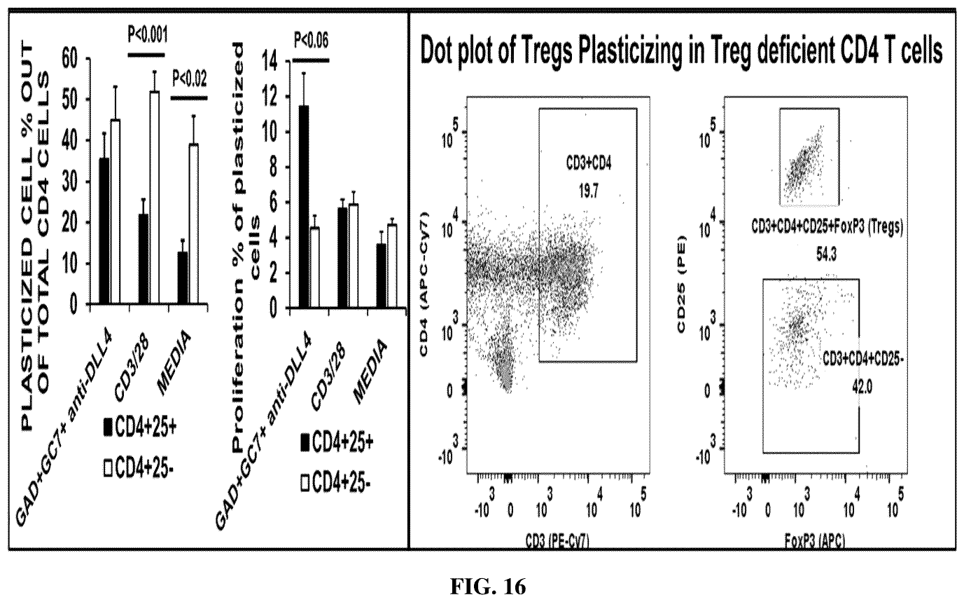

To evaluate the cell phenotype plasticizing under the influence of GC7+anti-DLL4, a Treg-deficient environment was created by sorting out CD4+CD25+cells from the CD4+ T cells using Miltenyi Biotec cell isolation kit. The Treg deficient CD4 T cell population were cultured in media supplemented with GC7+anti-DLL4+rhGAD65 and compared with conventional stimulation by anti-CD3/CD28 dyna beads and control media. After 7 days of culture, the plasticized CD4⁺CD25⁺ cells were quantified compared to the total CD4⁺CD25⁻ cells (Treg deficient) using flow cytometry. It was observed that 30-40% of CD4⁺CD25⁻ T cells plasticized into CD4⁺CD25⁺ cells (FIG. 16), in the GC7+anti-DLL4+rhGAD65 treated group. The proliferation % of plasticized CD4⁺CD25⁺ cells was also quantified in all treated groups and a significantly increased proliferation of CD4⁺CD25⁺ cells was found in the GC7+anti-DLL4+rhGAD65 treated group (FIG. 16). This experiment clearly revealed that the plasticizing cells belong to the CD4⁺CD25⁻ T cell lineage (FIG. 16).

Plasticized CD4⁺CD25⁺ Cells Expressed Treg Functional Phenotype and Maintained their Suppressive Capacity

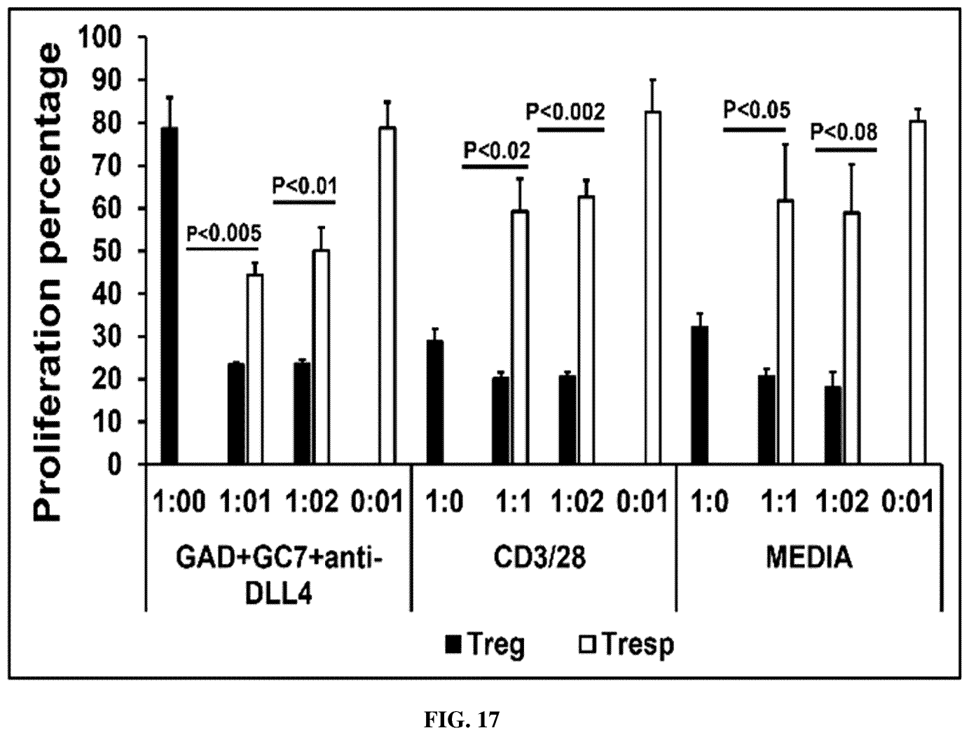

Next, to determine the functional phenotype of plasticized Tregs (CD4⁺CD25⁺), a suppression assay was designed with plasticized CD4⁺CD25⁺ and freshly isolated Tresp (CD4⁺CD25⁻) cells from PBMCs obtained from the peripheral blood of a LADA patient. The suppressive capacity of plasticized cells (CD4⁺CD25⁺) was evaluated by co-culturing them with freshly isolated autologous Tresp cells in Treg:Tresp ratios of 0:1, 1:1, 1:2 and 1:0 (FIG. 17). A flow cytometry analysis following the suppression assay (5 days post-co-culture) revealed that the plasticized cells (CD4⁺CD25⁺) expressed classical a Treg phenotype (CD4+CD25+Foxp3⁺) and suppressed the freshly isolated autologous T effector population (FIG. 17).

### No Adverse Effects of Immunomodulatory Treatment on Human CD4 Cells in Ex Vivo Experimentation

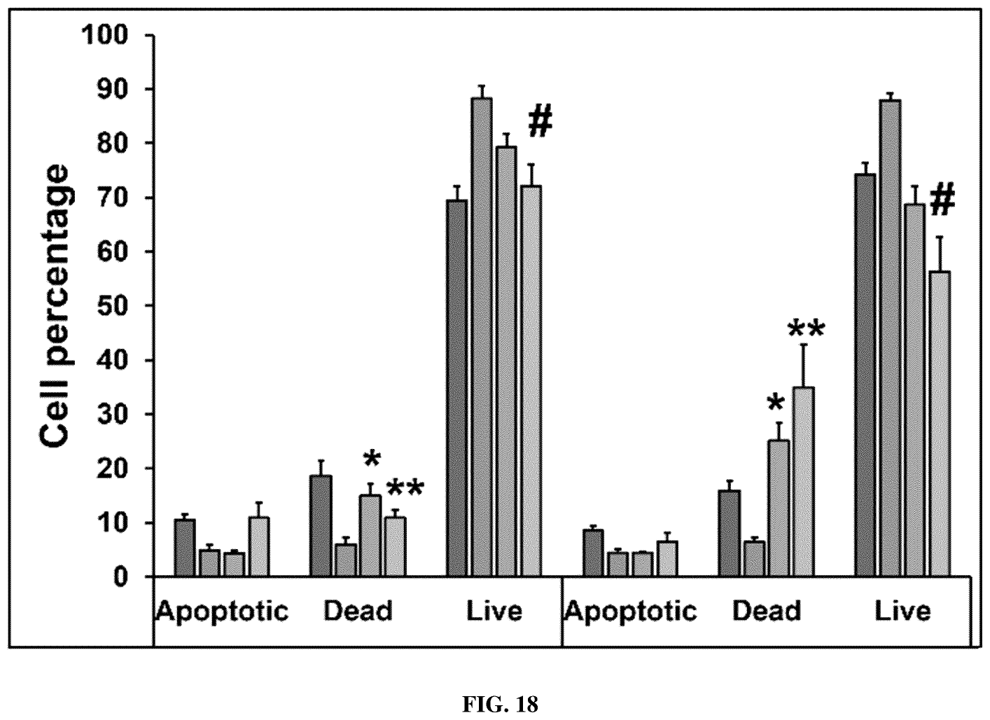

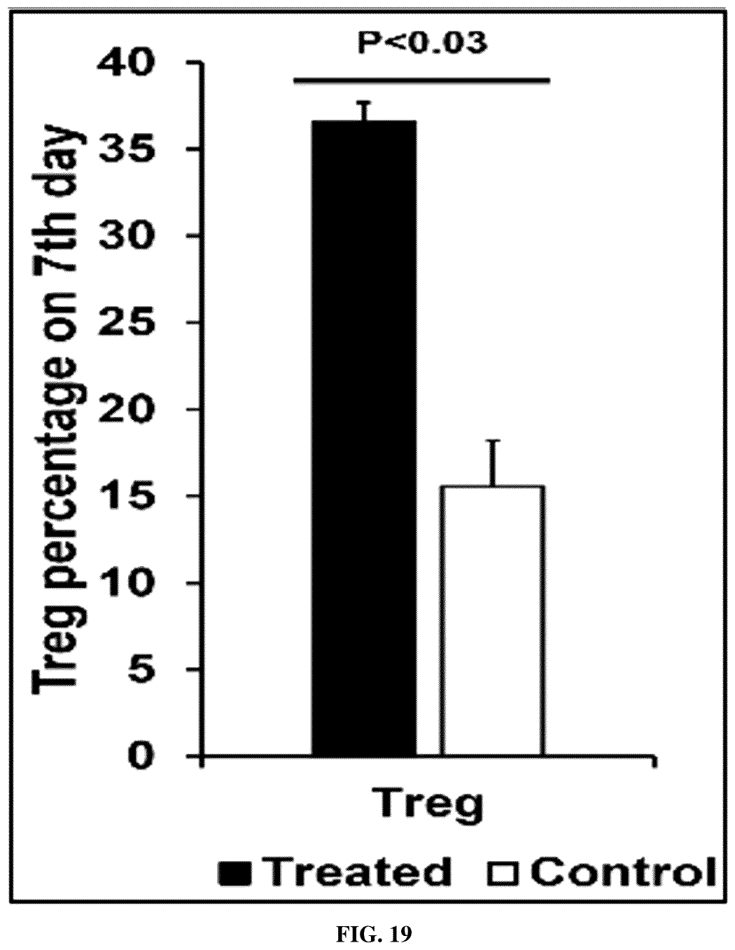

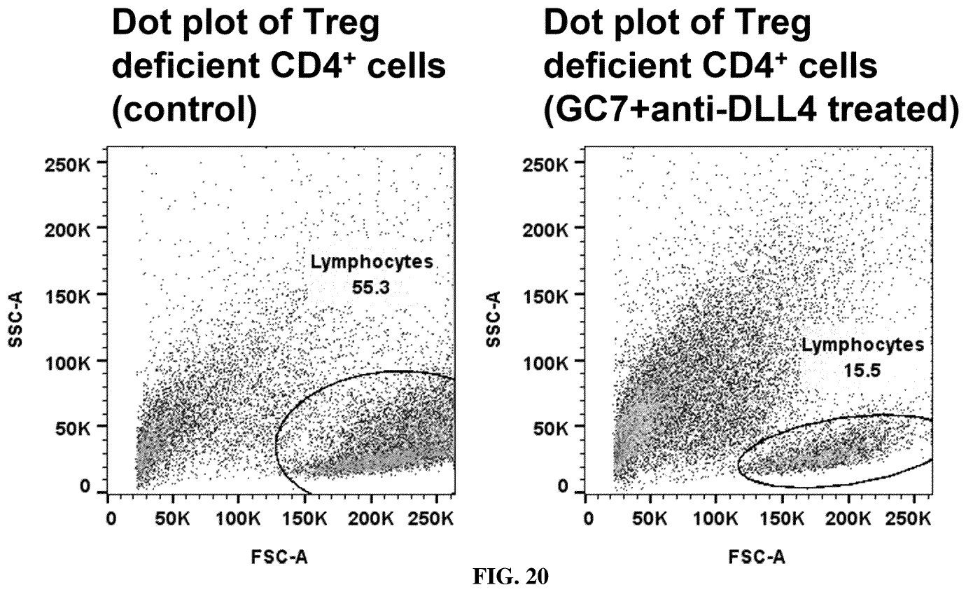

An experiment was designed to evaluate the feasibility of simultaneous inhibition in human studies and any adversaries. CD4⁺CD25⁻ cells were cultured in media/GC7+anti-DLL4+rhGAD65 as described in the plasticity experiment, and the live, dead, and apoptotic cells were quantified at 24 hr, 48 hr, 96 hr, and 7-day intervals. This longitudinal study provided a time-based assessment of cell viability and any apoptotic/off-target effects caused by simultaneous inhibition. There was no significant difference between treatment and control groups in terms of apoptotic, live, and dead cell counts in the first 24-48 hrs. However, an increase in dead cells post 96 hrs (grey) was observed in the treatment group from the conversion of CD4⁺CD25⁻ T cells into CD4⁺CD25⁺Tregs cells (plasticized cells), which suppressed the CD4⁺CD25⁻ T cell population (FIG. 18). This increase became even more pronounced on the 7^{th} day (yellow), owing to the continuation of plasticity and consequent suppression of CD4⁺CD25⁻ T cells. The signature of cells was further analyzed on the 7^{th} day using flow cytometry and verified an increased Treg (CD4⁺CD25⁺Foxp3⁺) population in the treated group due to the conversion of CD4⁺CD25⁻ cells to Tregs (FIG. 19). The effect of suppression can be easily seen on the lymphocyte population in treated group (15.5% live lymphocytes) whereas in control without any suppression, the lymphocyte population is 55.3% (live lymphocytes), both from the same human sample (FIG. 20).

Simultaneous Inhibition of eIF5A and Notch Signaling Induced Plasticity in CD4 Deficient PBMCs in Human Ex Vivo System

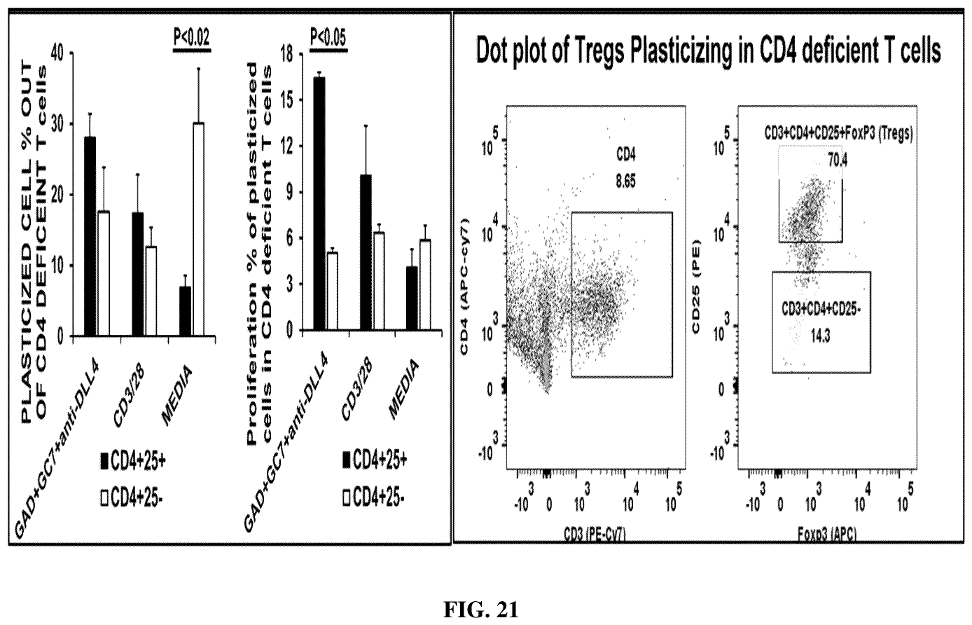

The plasticity of CD4 deficient PBMCs (PBMCs-CD4) isolated from LADA patients (n=3) and non-diabetic healthy donors (n=4) was investigated ex vivo. Patients showing a recent onset of LADA who were positive for GAD65 auto-antigen were recruited under the IRB-approved protocol. 10-20 cc blood was drawn and PBMCs were isolated using the FICOL gradient method. A CD4+ deficient environment was created by sorting out CD4+ T cells from PBMCs using Miltenyi Biotec cell isolation kit. The CD4+ deficient PBMCs were cultured in media supplemented with GC7+anti-DLL4+rhGAD65 and compared with conventional stimulation by anti-CD3/CD28 dyna beads and control media. After 7 days of culture, the plasticized CD4⁺CD25⁺ cells were quantified compared to the total CD4⁺CD25⁻ cells (Treg deficient) using flow cytometry. It was observed that 20-30% of CD4+ deficient PBMCs plasticized into CD4⁺CD25⁺ cells (FIG. 21) in the GC7+anti-DLL4+rhGAD65 treated group. The proliferation % of plasticized CD4⁺CD25⁺ cells was also quantified in all treated groups and a significantly increased proliferation of CD4⁺CD25⁺ cells was found in the GC7+anti-DLL4+rhGAD65 treated group (FIG. 21).

Plasticized CD4⁺CD25⁺ cells expressed Treg functional phenotype and maintained their suppressive capacity

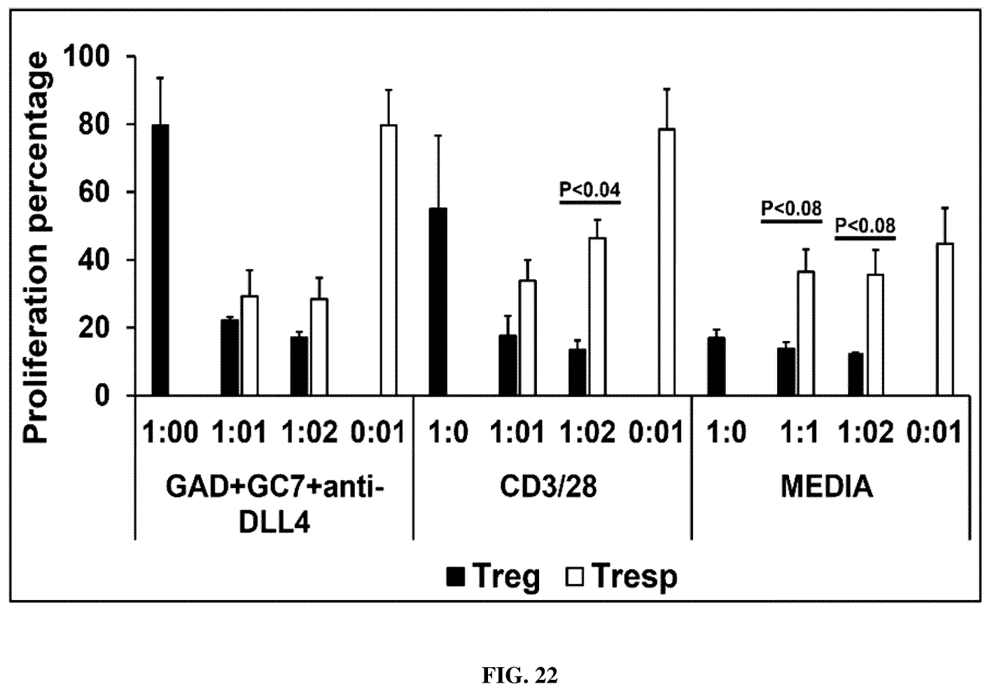

Next, to determine the functional phenotype of plasticized Tregs (CD4⁺CD25⁺) from CD4 deficient PBMCs, a suppression assay was designed with plasticized CD4⁺ CD25⁺ and freshly isolated Tresp (CD4⁺CD25⁻) cells from PBMCs obtained from the peripheral blood of same LADA patient. The suppressive capacity of plasticized cells (CD4⁺CD25⁺) was evaluated by co-culturing them with freshly isolated autologous Tresp cells in Treg:Tresp ratios of 0:1. 1:1. 1:2, and 1:0 (FIG. 22). A flow cytometry analysis following the suppression assay (5 days post-co-culture) revealed that the plasticized cells (CD4⁺CD25⁺) expressed classical a Treg phenotype (CD4+CD25+Foxp3⁺) and suppressed the freshly isolated autologous T effector population (FIG. 22).

No Adverse Effects of Immunomodulatory Treatment on Human CD4 Deficient PBMCs Cells (Including B Cells, Macropahges, Dendritic Cells, NK Cells, NKT Cells, CD8 Cells) in ex vivo experimentation

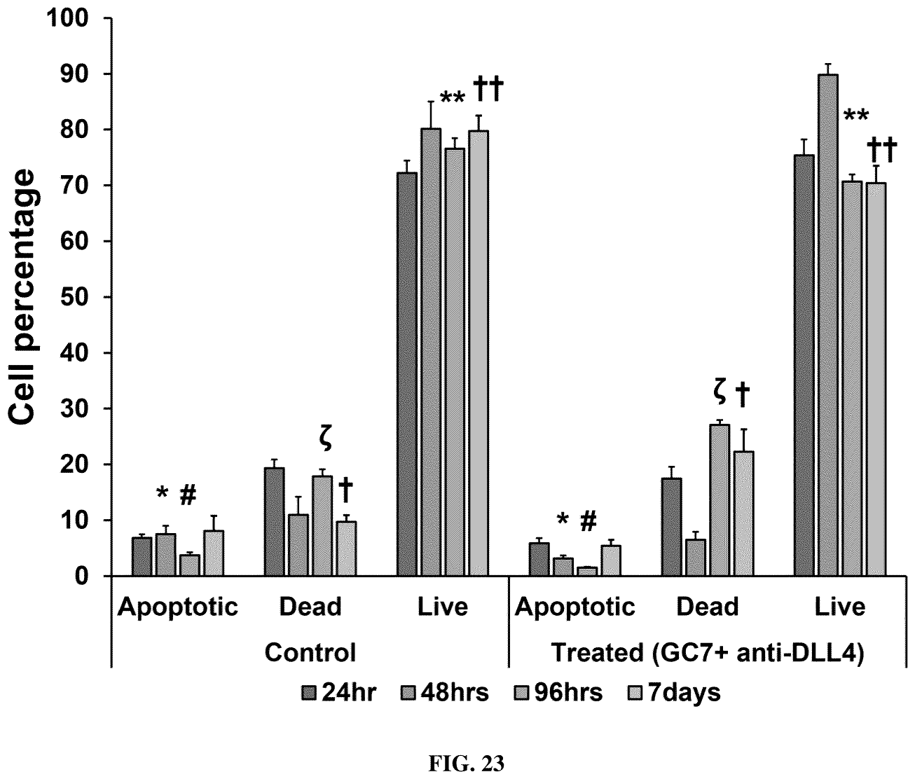

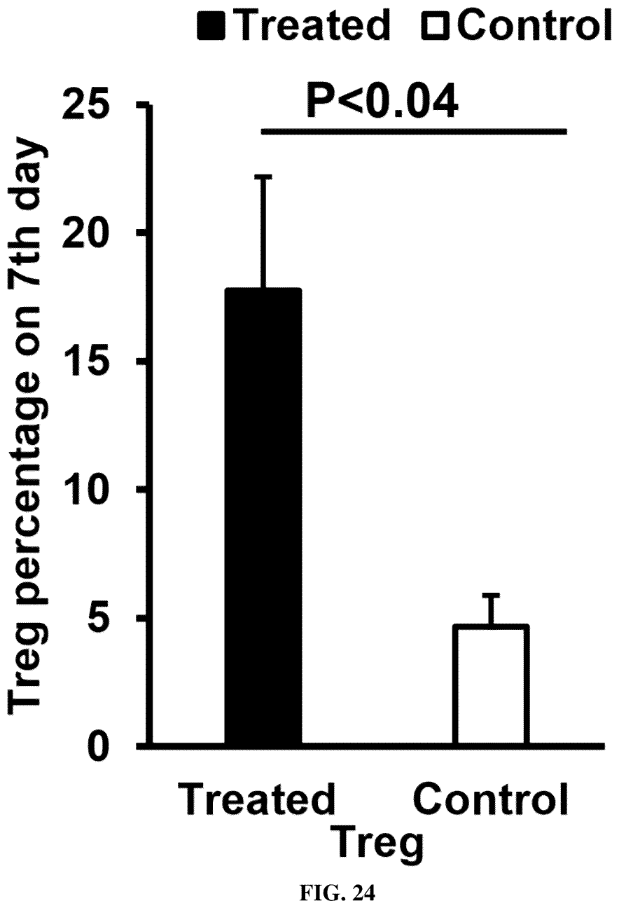

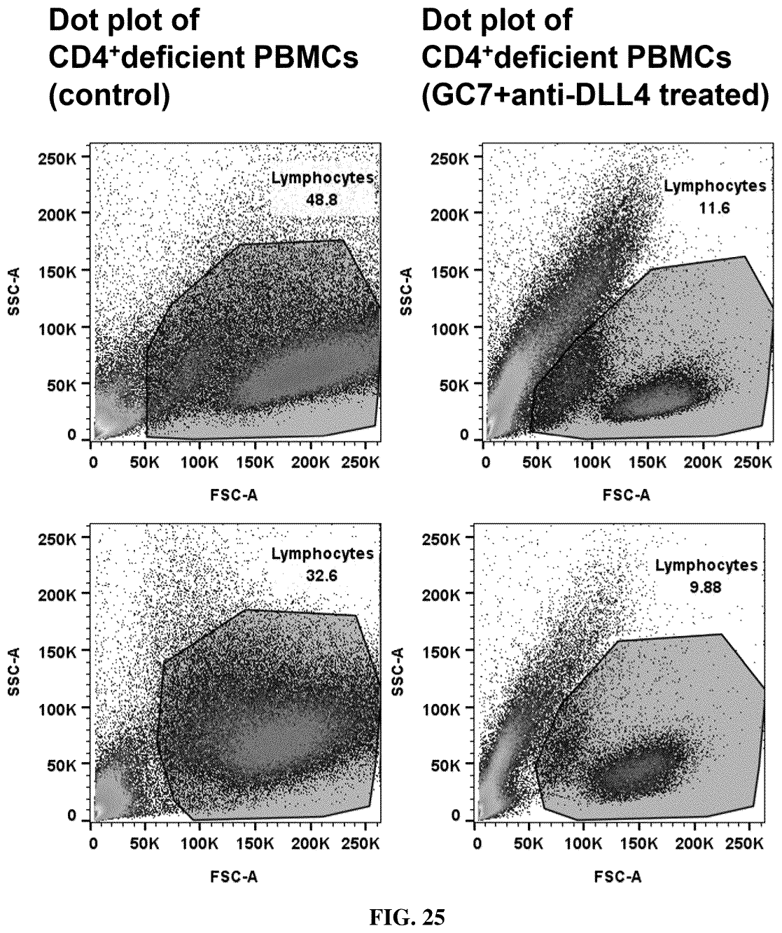

An experiment was designed to evaluate the feasibility of simultaneous inhibition in human studies and any adversaries. Similarly, CD4+ deficient PBMCs were cultured in media/GC7+anti-DLL4+rhGAD65 as described in the plasticity experiment and quantified the live, dead, and apoptotic cells at 24 hr, 48 hr, 96 hr, and 7 days intervalse. A significant increase in dead cells post 96 hrs was observed, similar to an earlier experiment with CD4+CD25− cells treated with GC7+anti-DLL4+rhGAD65 (FIG. 23). A significantly enriched population of Tregs on the 7th day of the experiment was also observed, indicating that these Tregs expressed a regulatory phenotype and therefore suppressed the Teffectors in the treated group, as evidenced by data analyzed in flow cytometry on the 7th day (FIG. 24). Also, the effect of suppression can be easily seen on the lymphocyte population in GC7+anti-DLL4+rhGAD65 treated group (11.6%, 9.88% live lymphocytes) whereas in the control from the same human sample, the lymphocyte population is 48.8%, 32.6% (live lymphocytes) without any suppression (FIG. 25). Therefore, it can be concluded that the dead cell number found in the control group is due to physiological cell death whereas the significantly increased dead cell count in the GC7+anti-DLL4+rhGAD65 treated group post 96 hr culture is due to enrichment of Tregs and consequent suppression of (CD4⁺ deficient PBMCs including CD8 T cells).

### Example IV

### Unconventional Intermediate Tregs in Autoimmune Diseases

Immune cell plasticity is the ability of immune cells to switch between functional states in response to the cytokine milieu. T cells change from their initial lineage commitment to different functional phenotypes in response to signals from the microenvironment, including cytokines, metabolites, hormones, toxins, and nutrients. The advancement in flow cytometry and single-cell sequencing led to the discovery and improved understanding of intermediate T-cell subsets. Indeed, in addition to conventional T effector cells and T regulatory cells. T cells expressing a plethora of transcription factors alone or in combination, viz. FOXP3+, FOXP3+IL-17+, FOXP3+IFNg+, and FOXP3+IL-17+IFNg+, are encountered at the site of inflammation. Overall, it is a fine balance of pro-inflammatory and anti-inflammatory cells, which, under autoimmune pathologic conditions, creates a cytokine milieu that promotes the conversion of Tregs into intermediate subsets (pro-inflammatory type). In autoimmune conditions, Tregs exhibit abnormality in number, function, and expression profile, and transdifferentiate into a proinflammatory phenotype. In type 1 diabetes (T1D), Tregs undergo increased apoptosis, exhibit an unstable FOXP3 expression, and an increase in the frequency of intermediate Tregs that produce proinflammatory cytokines, such as IFNg and IL-17. Pathological conversion of Tregs that secrete IFNg or T helper (Th)17 (intermediate state) establishes the importance of intermediate Treg cell types and the plastic fate of Tregs in autoimmune diabetes, experimental autoimmune encephalitis (EAE), and autoimmune arthritis.

In this example, an intermediate Treg population expressing proinflammatory cytokines in T1D/LADA patients is described. This highly plastic intermediate subset originates from CD4+CD25− T cells. Further, the conversion of these cells into_is described. This is a possible therapeutic intervention in T1D/LADA, where diabetogenic proinflammatory Treg (intermediate) cell subset can be driven to a regulatory T cell phenotype (CD3⁺CD4⁺CD25⁺FOXP3⁺) by simultaneous inhibition of eIF5a and Notch pathways.

Peripheral blood (10-20 ml) was collected in heparinized tubes, and mononuclear cells (PBMCs) were isolated with the Ficoll density gradient method. CD4+ T cells were isolated from PBMCs using a CD4+ T cell isolation kit (Miltenyi Biotech, USA, #130-096-533). Briefly, untouched CD4+ T cells were column purified using a cocktail of biotin-conjugated antibodies against non-CD4+ T cells. Thus, all non-CD4+ T cells, i.e., CD8+ T cells, monocytes, neutrophils, dendritic cells, NK cells etc., were retained in the magnetic column while untouch ed purified CD4+ T cells were collected in flowthrough. Column-purified CD4+ T cells were further subjected to MACS column sorting based on CD25+ positivity using a Treg isolation kit (Miltenyi Biotech, USA). The retained cells in the column containing CD4+CD25+ T cells and flowthrough containing CD4+CD25− T cells were eluted. A portion of the eluted cells from column and flowthrough was stained with APC-Cy7-CD4 (cat #557871) and PE-CD25 (cat #555432) (BD Biosciences) and acquired at flow cytometry and analyzed by Flow Jo (BD Biosciences) software for confirmation. CD4+CD25+ T cells and CD4+CD25− T cells were permeabilized with cytofix/cytoperm fixation and permeabilization solution (BD Biosciences) for intracellular staining. Intracellular staining was performed with fluorochrome-conjugated antibodies against human PE-IL17 (cat #560486), APC-IFNg (cat #25723.11) and Alexa Fluor 488/Alexa Fluor 647-forkhead box P3 (FOXP3) (cat #560047,560045). CD4+CD25+ T cells and CD4+CD25− T cells were analyzed by flow cytometry. The data were analyzed using Flow Jo (BD Biosciences) software, and CD4+IFNg+IL17+FOXP3+ were quantified from CD4+CD25+ and CD4+CD25− T cell subsets.

### Methods

Peripheral blood from patients with T1D/LADA (n=6) and healthy adult controls (n=7) was used to isolate the CD25 deficient CD4+ (CD4+CD25−) T cell population and CD4 deficient peripheral blood mononuclear cells (PBMCs). Cells were subjected to immunomodulatory treatment for 7 days with GAD65+GC7+anti-DLL4 and compared with conventional anti-CD3/CD28/CD137 stimulation for conversion into regulatory T cell phenotype (CD4+CD25+FOXP3+). The newly plasticized T regulatory cells (Tregs) from the CD4+CD25− T cell population and CD4 deficient PBMCs were assessed for their suppressive potential against freshly isolated autologous responder T cells (Tresp, CD4+CD25−). Also, live, dead, and apoptotic cell counts were performed to evaluate the adverse effects of immunomodulatory treatment on immune cells.

### Results

Patients with T1D/LADA had a significantly increased number of intermediate proinflammatory CD4+CD25−IFNg+IL17+FOXP3+ Treg cells that proliferated and plasticized into stable Tregs (CD4+CD25+FOXP3+) on immunomodulatory treatment. Here, it is shown that simultaneous inhibition of eIF5a and Notch plasticized Treg deficient CD4+ (CD4+CD25−) T cells and CD4 deficient PBMCs into CD4+CD25+FOXP3+ Tregs, withstanding a proinflammatory milieu mimicking T1D/LADA. The newly plasticized Tregs had a stable and suppressive functional phenotype. Further, the immunomodulatory treatment had no adverse effect on the immune cells.

This example identified a population of CD4+CD25−IFNg+IL17+FOXP3+ Treg cells in patients with T1D/LADA and successfully reverted it to functionally active Treg cells with GAD65+GC7+anti-DLL4 treatment, capable of suppressing the diabetogenic T effector cells. The present approach is a multipronged approach involving inhibition of eIF5a and Notch pathways that address the upregulation of immune tolerance, differentiation, and proliferation of cytotoxic T cells.

T1D/LADA is an autoimmune disorder in which Tregs are either too low in number or are unstable, hence insufficient to contain the immune assault of cytotoxic T lymphocytes on islet beta cells. Current immunotherapies, including polyclonal expansion of Tregs and the use of low-dose IL-2, TGF-β therapy, have inherent limitations of inadequate number of Tregs in patients for in vitro expansion, or bystander paradoxical response, and inability to induce Tregs proliferation in proinflammatory cytokine milieu.

Patients with T1D/LADA have a significantly increased number of proinflammatory cytokineexpressing Treg cells (CD4+CD25−IFNg+IL17+FOXP3+). This intermediate cell population successfully reverted to the stable Treg phenotype using GAD65+GC7+anti-DLL4 immunomodulatory treatment.

The current treatment strategies for T1D/LADA are based on insulin replacement; however, there is an unmet need to address the underlying immune imbalance and enrich the Tregs. In the present example, the Tregs were induced in a proinflammatory milieu that mimicked T1D/LADA, and the newly plasticized Tregs had a functional regulatory phenotype capable of suppressing the diabetogenic T effector cell population. This urges further investigation into the applicability of this therapeutic intervention for future strategies in the immunotherapy of T1D/LADA and other autoimmune disorders.

### Introduction

Immune cell plasticity is the ability of immune cells to switch between functional states in response to the cytokine milieu. An ample body of literature exists providing empirical support to the idea that T cells change from their initial lineage commitment to different functional phenotypes in response to signals from the microenvironment, including cytokines, metabolites, hormones, toxins, and nutrients. The advancement in flow cytometry and single-cell sequencing led to the discovery and improved understanding of intermediate T-cell subsets. Indeed, in addition to conventional T effector cells and T regulatory cells, T cells expressing a plethora of transcription factors alone or in combination, viz. FOXP3+, FOXP3+IL-17+, FOXP3+IFNg+, and FOXP3+IL-17+IFNg+, are encountered at the site of inflammation. Overall, it is a fine balance of proinflammatory and anti-inflammatory cells, which, under autoimmune pathologic conditions, creates a cytokine milieu that promotes the conversion of Tregs into intermediate subsets (proinflammatory type). In autoimmune conditions, Tregs exhibit abnormality in number, function, and expression profile, and transdifferentiate into the proinflammatory phenotype. In T1D. Tregs undergo increased apoptosis, and exhibit an unstable FOXP3 expression and an increase in the frequency of intermediate Tregs that produce proinflammatory cytokines, such as IFNg and IL-17. Pathological conversion of Tregs that secrete IFNg or T helper (Th) 17 (intermediate state) establishes the importance of intermediate Treg cell types and the plastic fate of Tregs in autoimmune diabetes, experimental autoimmune encephalitis (EAE), and autoimmune arthritis. In the present example, this flexible behavior exhibited by Tregs in autoimmune conditions was exploited to revert them to a nonpathogenic stable Treg state using the immunomodulators N1-guanyl-1,7-diaminoheptane (GC7) and anti-Delta-Like Ligand 4 (DLL4).

GC7 is the most potent inhibitor of deoxyhypusine synthase (DHS), an enzyme responsible for the hypusination (post-translational addition of amino acid hypusine) and functional activation of eukaryotic initiation factor 5a (eIF5a), eIF5a is a 17 kDa conserved protein initially identified as a translational factor. However, its function is context-dependent and is responsible for nucleocytoplasmic shuttling of specific proteins and mRNAs primarily associated with proinflammatory markers, e.g., iNOS, IFNg, dendritic cell maturation marker CD83, M1 macrophage hallmarks. Interestingly, the gene encoding eIF5a is found in diabetes susceptibility loci in mice as well as in humans. Hypusinated eIF5a (hyp-eIF5a) is overexpressed in T1D, contributing to the proinflammatory cytokine milieu and exacerbating endoplasmic reticulum (ER) stress. Hyp-eIF5a inhibition using GC7 has previously confirmed the impairment of proinflammatory polarization of Th1 immune cells and cytokinemediated dysfunction in NOD mice, the spontaneous humanized mice model of T1D, and human islets in vitro. Notch is a conserved cell-to-cell signaling network that has a prominent role in the maturation, activation, and differentiation of T cells. Notch ligand DLL4 inhibits the JAK3/STATS activation pathway necessary for FOXP3 expression and maintenance. Inhibition of Notch signaling with anti-DLL4 results in alternative differentiation and expansion of Tregs in NOD and humanized T1D mice as well as in EAE. Further, Notch signaling has a critical role in Treg differentiation, independent of the thymus, and is an essential regulator of RORγt and IL-23r genes, leading to differentiation and activation of Th17 cells. Thus, Notch and eIF5a support a proinflammatory phenotype, and therapeutic interventions in inhibiting this cascade could reset the immune imbalance in T1D/LADA and other autoimmune diseases.

In this example, the significant presence of a CD4+CD25−IFNg+IL17+FOXP3+ intermediate Treg cell subset in patients with recent onset of T1D/LADA is described. These cells were enriched and plasticized into Tregs by GC7 and anti-DLL4 treatment. The newly plasticized Tregs had a stable CD25+FOXP3+ expression and suppressed autologous T effector cells. Moreover, GC7 and anti-DLL4 treatment had no adverse effects on the immune cells, demonstrating the efficacy of simultaneous inhibition of Notch and eIF5a for restoring immune imbalance in T1D/LADA as well as in other autoimmune disorders.

### Methods

### Patient Recruitment

Healthy adults (n=7) and patients with T1D/LADA (n=6) were enrolled in this study approved by the Institutional Review Board of the University of Toledo, College of Medicine, and Life Sciences (#301804-UT). Minors (age <18 years of age) and patients with a history of T1D/LADA>5 years were excluded from the study. All subjects signed informed consent to participate in the present protocol. The study procedures included recording the history of their disease, as per the time of onset, the baseline HbAlc measurement and serum titers of GAD65 autoantibody upon diagnosis, and the mean daily insulin dosing requirements. In addition, height and weight measurements were performed, and the body mass index (BMI) was calculated based on the following formula: BMI=Weight/(Height)².

Characterization of Intermediate Treg Cells from Patients with T1D/LADA and Healthy Adult Donors

Peripheral blood (10-20 ml) was collected in heparinized tubes, and mononuclear cells (PBMCs) were isolated with the Ficoll density gradient method. CD4+ T cells were isolated from PBMCs using a CD4+ T cell isolation kit (Miltenyi Biotech, USA, #130-096-533). Briefly, untouched CD4+ T cells were column purified using a cocktail of biotin-conjugated antibodies against non-CD4+ T cells. Thus, all non-CD4+ T cells, i.e., CD8+ T cells, monocytes, neutrophils, dendritic cells, NK cells, etc., were retained in the magnetic column and were eluted from the column and considered as CD4 deficient PBMCs while untouched purified CD4+ T cells were collected in flowthrough. Column-purified CD4+ T cells were further subjected to MACS column sorting based on CD25+ positivity using a Treg isolation kit (Miltenyi Biotech, USA). The retained cells in the column containing CD4+CD25+ T cells and flowthrough containing CD4+CD25− T cells were eluted. A portion of the eluted cells from column and flowthrough was stained with APC-Cy7-CD4 (cat#557871) and PE-CD25 (cat#555432) (BD Biosciences) and acquired at flow cytometry and analyzed by Flow Jo (BD Biosciences) software for confirmation. CD4+CD25+ T cells and CD4+CD25− T cells were permeabilized with cytofix/cytoperm fixation and permeabilization solution (BD Biosciences) for intracellular staining. Intracellular staining was performed with fluorochrome-conjugated antibodies against human PE-IL17 (cat#560486), APC-IFNg (cat#25723.11) and Alexa Fluor 488/Alexa Fluor 647-forkhead box P3 (FOXP3) (cat#560047,560045). CD4+CD25+ T cells and CD4+CD25− T cells were analyzed by flow cytometry. The data were analyzed using Flow Jo (BD Biosciences) software, and CD4+IFNg+IL17+FOXP3+ were quantified from CD4+CD25+ and CD4+CD25− T cell subsets.

Plasticization of Treg deficient CD4+ (CD4+CD25−) T cells and CD4 deficient PBMCs

The isolated CD4+CD25− T cells were incubated in Celltrace CFSE dye (Thermo Fischer Scientific, cat #C1157) for 20 minutes to trace proliferation during the immunomodulatory treatment (n=4, in triplicate repeated thrice). After thorough washing, CD4+CD25− T cells were separated into batches for individual treatments. One batch of cells was suspended in RPMI 1640 medium (Gibco, cat #11875093) supplemented with 20% Fetal calf serum (FCS, Thermo Fischer Scientific), GAD65 (Kronus, star, ID, 4 μg/ml), GC7 (Chemcruz SC 396111, 100 μM/ml), and anti-DLL4 (Invitrogen, MHD4-46, 10 μg/ml). The second batch was suspended in RPMI 1640 medium supplemented with 20% FCS and stimulated by conventional T activator CD3/CD28/137 beads (Gibco cat #11162D). The third batch of cells was suspended in an RPMI 1640 medium supplemented with 20% FCS and used as a control (media group). The CD4+CD25− T cells were maintained at 37° C. and 5% CO₂ for 7 days and investigated for T cell plasticity. After 7 days of culture, cells in each group were quantified by flow cytometry for the presence of CD4+CD25− and CD4+CD25+ T cells and their respective proliferation was quantified on Flow Jo (BD Biosciences) software.

Similarly, CD4 deficient PBMCs were also incubated in Celltrace CFSE dye for 20 minutes, washed thoroughly, and later divided into 3 groups supplemented with RPMI+20% FCS. One batch of cells was treated with GAD65(4 μg/ml), GC7 (100 μM/ml), and anti-DLL4 (10 μg/ml), another batch was stimulated by conventional T cell activator CD3/CD28/CD137 beads, while control was suspended in RPMI+20% FCS only. The cells were maintained at 37° C. and 5% CO₂ for 7 days and investigated for T cell plasticity. After 7 days, CD4 deficient PBMC cultures were analyzed by flow cytometry. Cells in each group were first gated for CD4+ T cells; later, CD4+ T cells were gated for CD4+CD25− and CD4+CD25+ T cells. The proliferation of CD4+CD25− and CD4+CD25+ T cells was quantified on Flow Jo (BD Biosciences) software.

### Suppression Assay of Plasticized Tregs vs Autologous Naïve T Responsive Cells

Plasticized CD4+CD25+ Treg cells were MACS column purified from the cells cultured for 7 days under the influence of GAD65+GC7+anti-DLL4, conventional anti-CD3/CD28/CD137 stimulation, and culture media (RPMI+20% FCS), using a CD4+CD25+ Treg isolation kit as previously described. At the same time, CD4+CD25− T cells (Tresp cells) were isolated from autologous patients' freshly withdrawn peripheral blood, as described earlier. A suppression assay (n=4, in triplicate repeated thrice) was undertaken using 1:0, 1:1, 1:2, and 0:1 ratios of plasticized CD4+CD25+ cells (Tregs) from all three treatment groups: naïve/fresh CD4+CD25− (Tresp) cells. Before co-culture, the CD4+CD25+ cells (Tregs) and CD4+CD25− T cells (Tresp cells) were incubated with CFSE to trace the proliferation of the cells. The cells were thoroughly washed and cultured in different ratios for 5 days in RPMI+20% FCS culture media. After 5 days, cells were stained with anti-human CD4 and CD25 immunofluorescent antibodies, and gated Treg (CD4+CD25+) and Tresp (CD4+CD25−) cell populations were traced by CellTrace (CFSE-FITC, xaxis) for proliferation using Flow Jo (BD Biosciences) software. Similarly, CD4+CD25+ Treg cells that plasticized from CD4 deficient PBMCs cultured for 7 days under the influence of GAD65+GC7+anti-DLL4, conventional anti-CD3/CD28/CD137 stimulation, and culture media were also column purified using CD4+CD25+ Treg isolation kit. An in vitro suppression assay (n=4, in triplicate repeated thrice) was performed to investigate the suppressive phenotype of newly plasticized CD4+CD25+ Treg cells. Here also, column-purified plasticized Tregs from GAD65+GC7+anti-DLL4, conventional anti-CD3/CD28/CD137 stimulation, and culture media (control) groups were co-cultured with freshly isolated CD4+CD25− Tresp cells from autologous patients in different Treg: Tresp ratios of 1:0, 1:1, 1:2 and 0:1. Before co-culture, the CD4+CD25+ cells (Tregs) and CD4+CD25− T cells (Tresp cells) were incubated with CFSE to trace the proliferation of cells. The cells were thoroughly washed and cultured in different ratios for 5 days in RPMI+20% FCS culture media. After 5 days of co-culture, cell proliferation was traced by CFSE dye, and the Treg phenotype was confirmed using flow cytometry as described earlier.

Assessment of Live, Dead, and Apoptotic Cell Populations after Immunomodulatory Treatment

Treg deficient CD4+ (CD4+CD25−) cells and CD4 deficient PBMCs (n=3 in triplicate, repeated twice) were assessed for live, dead, and apoptotic cells after immunomodulatory treatment. One batch of cells was suspended in RPMI 1640 medium supplemented with 20% FCS, GAD65 (4 μg/ml), GC7 (100 μM/ml), and anti-DLL4 (10 μg/ml), while another batch was suspended in RPMI 1640 culture medium supplemented with 20%FCS and served as untreated control. Cells were cultured for 7 days and then harvested to quantify live, dead, and apoptotic cells. Live, dead, and apoptotic cell populations were quantified using PO-PRO-1 and 7 amino actinomycin D (Invitrogen cat #V35123) as described in the protocol. The cells were quantified by flow cytometry and analyzed using Flow Jo (BD Biosciences) software. Apoptotic cells show violet fluorescence, dead cells show violet and red fluorescence, and live cells show little or no fluorescence.

### Flow Cytometry Protocol

All the flow cytometric analyses were done on at least 25000 live cells. For flow cytometry experiments involving cell surface and intracellular staining, the established immunofluorescence staining protocol was executed. Briefly, cells were stained with fluorochromeconjugated antibodies against human PECy7-CD3 (cat#557851), APCCy7-CD3 (cat#557832), APC-Cy7-CD4 (cat#557871), PECy5-CD8 (cat#555636), PE-CD25 (cat#555432) (BD Biosciences). A subset of cells was permeabilized with cytofix/cytoperm fixation and permeabilization solution (BD Biosciences). Intracellular staining was performed with fluorochrome-conjugated antibodies against human PE-IL17 (cat#560486), APC-IFNg (cat#25723.11), and Alexa Fluor 488/Alexa Fluor 647-forkhead box P3 (FOXP3) (cat#560047,560045). The data were analyzed using Flow Jo (BD Biosciences) software.

### Statistical Analysis

Statistical analysis was performed using SAS MIXED procedure (version 9.4, SAS Institute, Inc.). Data were tested for normality with the Kolmogorov-Smirnov test and transformed to natural logarithms and ranks as appropriate when not normally distributed. Multiple groups were analyzed by 1- or 2-way ANOVA with Tukey's multiple comparison test, while differences between the two treatments were compared by a paired comparison (two-tailed) T-test. The statistical significance threshold was set at P≤0.05. Data are presented as the means±standard error of means (SEM).

### Results

CD4+CD25−IFNg+IL17+FOXP3+ Intermediate Treg Cells are Significantly Increased in Patients with Recent-Onset T1D/LADA

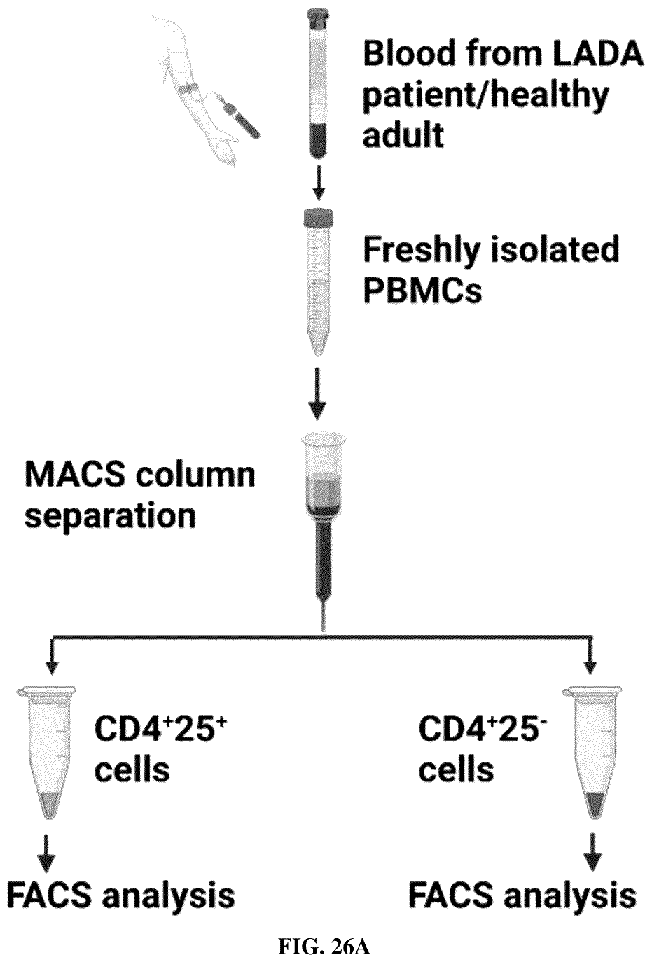

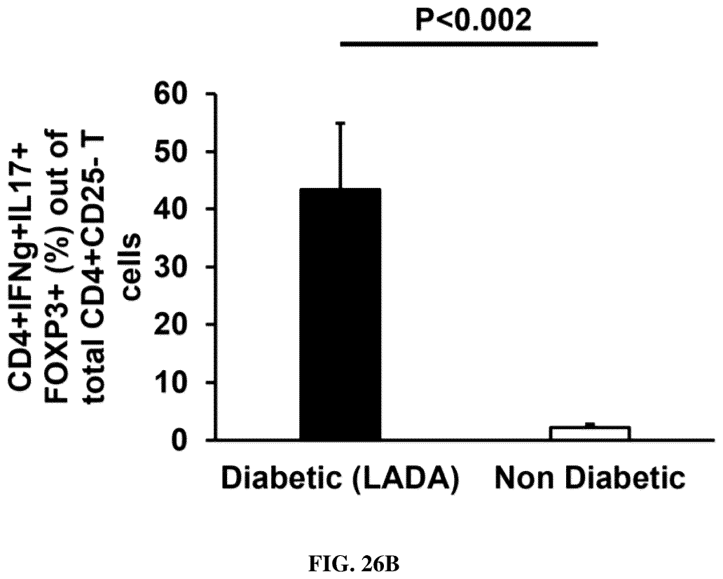

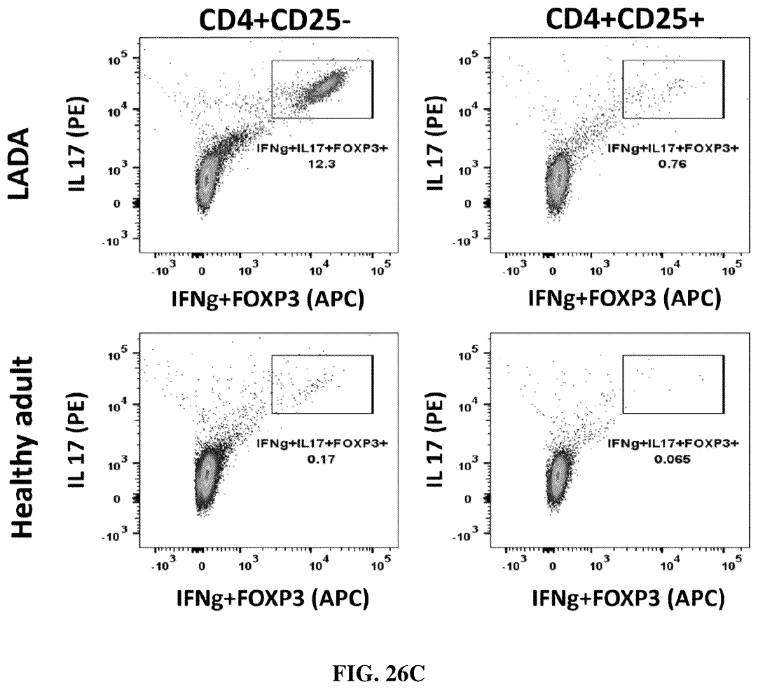

The baseline characteristics of the patients with recent onset of T1D/LADA (n=6) and the healthy controls (n=7) are presented in Table 1. Their age and BMI did not differ significantly (t-test P value >0.50 for both comparisons). Phenotypic immune cell profiling of CD4+CD25+and CD4+ CD25− T cell subsets revealed a significantly increased number of CD4+ T cells expressing IFNg+IL17+FOXP3+ (43.4±11.6% of cells) in Treg deficient (CD4+CD25−) T cell pool of patients with T1D/LADA while 2.23±0.6% were present in healthy controls (FIGS. 26A-26C). This finding is instrumental since in healthy human adults, the percentage of CD4+ T cells expressing FOXP3 in peripheral blood is about 8.6±1.7%, of which 3.2±1.1% express IL-17. These CD4+CD25− expressing IFNg+IL17+FOXP3+ cells can be deemed as transient Tregs (intermediate differentiation subset of unfit Tregs that have acquired T effector (Teffs) phenotype and/or not terminally differentiated) or effector T cells expressing FOXP3 since human effector T cells (Teffs) transiently express intermediate levels of FOXP3. The plasticization of this intermediate population of CD4+CD25−IFNg+IL17+FOXP3+ T cells into Tregs by inhibition of eIF5a and Notch pathways based on the previous studies described in the examples above was then undertaken.

GAD65+GC7+anti-DLL4 Treatment Plasticizes CD4+CD25− T Cells into CD4+CD25+ T Cells

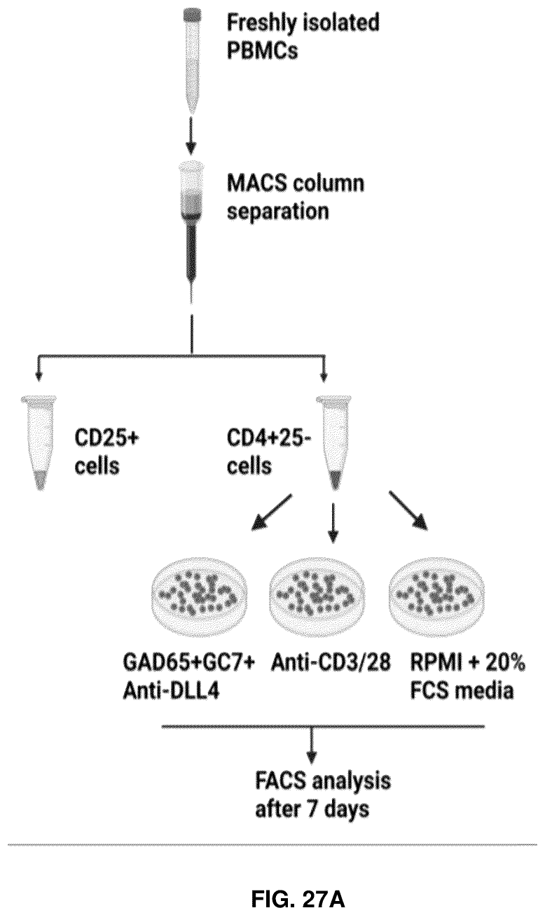

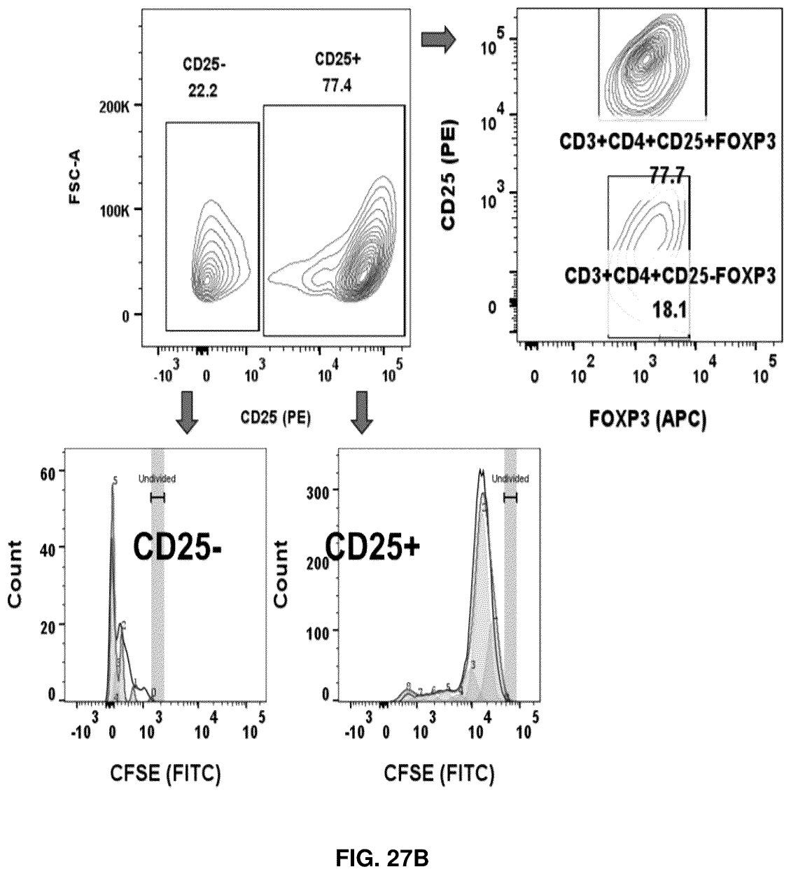

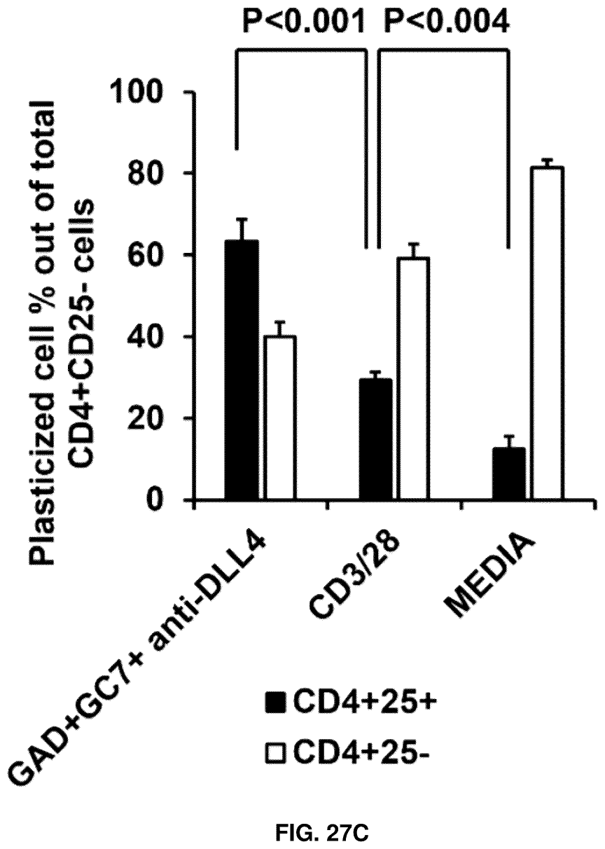

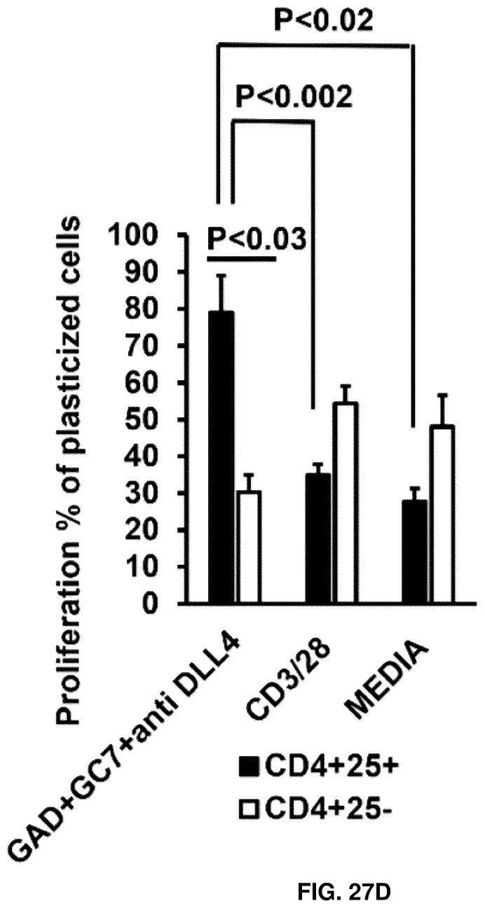

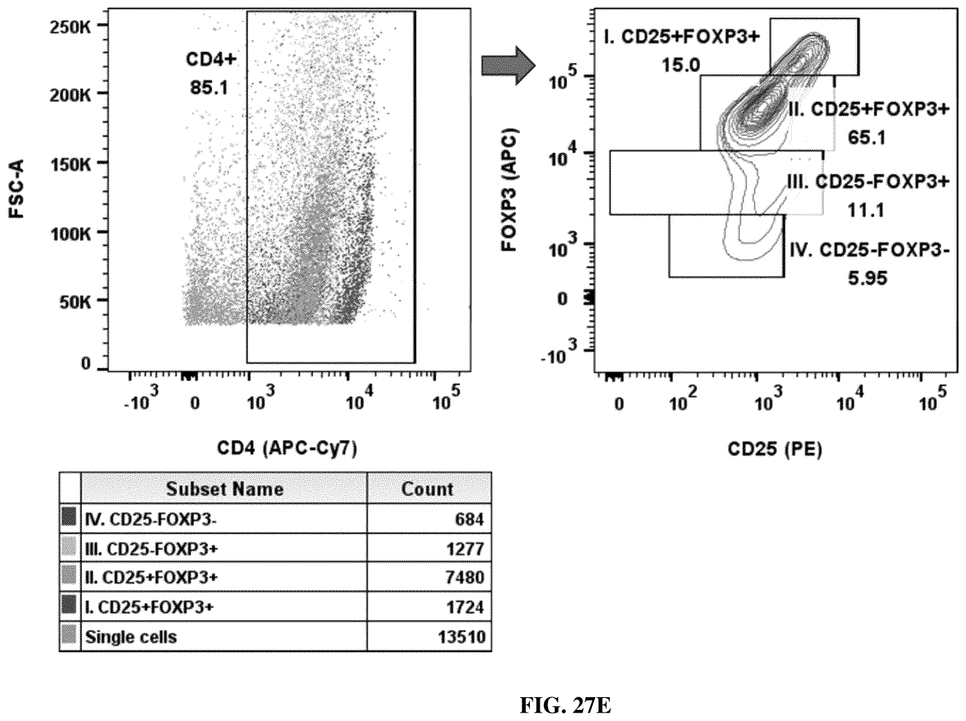

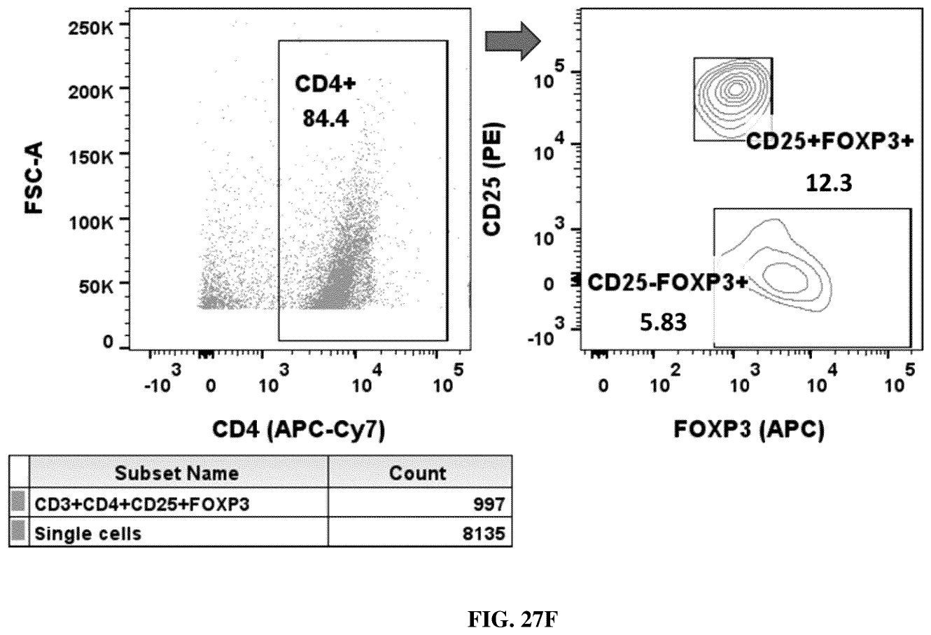

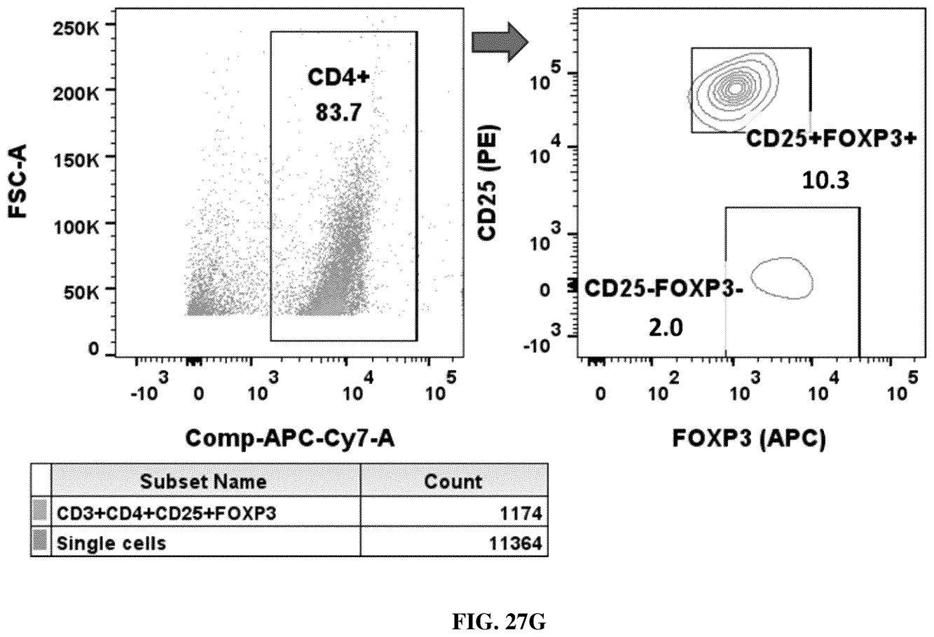

To further ascertain the origin of CD4+CD25−IFNg+IL17+FOXP3+ T cells and induce plasticity in the intermediate cells, PBMCs from patients with recent-onset T1D/LADA were again isolated. The pure population of CD4+ T cells was sorted using a CD4 T cell isolation kit and CD25+ T cells were further purified from CD4+ T cells. Thus, the CD4+cell pool was rendered completely devoid of CD25+ Tregs and it was subjected to simultaneous immunomodulatory treatment with GC7 and anti-DLL4 in the presence of rhGAD65 protein (FIG. 27A). Any CD4+CD25+ cells in this cell pool must have a CD25− origin, and the immunomodulatory treatment rewires the CD4+CD25−IFNg+IL17+FOXP3+ T cells into CD4+CD25+FOXP3+ T cells. After seven days of immunomodulatory treatment, a significantly increased number of CD4+CD25+ T cells was identified in the GAD65+GC7+anti-DLL4 treated group, indicating CD4+CD25− T cells undergo plasticity changes and are in the process of expressing a T regulatory cell phenotype (CD3+CD4+CD25+FOXP3+) (FIG. 27B). As is evident in a representative FIG. 27E, four types of cell populations are present in CD4+CD25− T cells in the GAD65+GC7+anti-DLL4 treated group post 7-day treatment viz. CD25−FOXP3− (IV), CD25−FOXP3+ (III), CD25+FOXP3+ (II), and CD25+highFOXP3+high(I), wherein CD25− T cells (III, IV, green) are transitioning into CD25+FOXP3+ (II. orange color) and CD25+highFOXP3+high (I. red color). Therefore, in the antigen-specific GAD65+GC7+anti-DLL4 treated group, a significant number of CD4+CD25− T cells (63.4±5.4%) plasticized into CD4+CD25+ T cells out of total CD4+CD25− T cells (FIG. 27C) and had significantly higher proliferative capacity (FIG. 27D). On the contrary, in the conventional anti-CD3/28/137 stimulation group, 29.3±2.0% of CD4+CD25− T cells plasticized into CD4+CD25+ T cells (FIG. 27C); however, the proliferative capacity of CD4+CD25− T cells plasticizing into CD4+CD25+ T cells was significantly less (P<0.002) compared to GAD65+GC7+anti-DLL4 treated group (FIG. 27D). Moreover, around 12.5±3.1% of CD4+CD25− T cells in the culture media (RPMI+20% FCS) group also plasticized into CD4+CD25+ T cells (FIG. 27C). Since Tregs constitute only a small fraction of T cells in human peripheral blood (5%), in the culture media (control group) where cells were cultured in a neutral environment (RPMI+20% FCS), these “intermediate” cells were able to revert to their original Treg phenotype. This indicates that the process of differentiation from CD4+CD25− T cells to CD4+CD25+ T cells is a phenomenon of cellular homeostasis but, in response to a proinflammatory milieu of T1D/LADA these cells are not able to sustain the Treg phenotype. This is a widely observed event in T1D/LADA and other autoimmune diseases where Tregs exhibit unstable FOXP3 expression and pathologic conversion of FOXP3+ into FOXP3+1L17+ and FOXP3+IFNg+ T cells occur.

The differential enrichment and plasticization of CD4+CD25− T cells into CD4+CD25+ T cells was further investigated in GAD65+GC7+anti-DLL4, conventional anti-CD3/28/137 stimulation, and culture media conditions. The proliferation index of cells undergoing plasticity was assessed using a CFSE dye after a 7-day treatment (FIG. 27D). The percentage proliferation of plasticized CD4+CD25+ T cells was significantly higher in the GAD65+GC7+anti-DLL4 treatment group compared to other groups (79.0% in the GAD65+GC7+anti-DLL4 group, 34.8%in anti CD3/28/137 stimulation, 27.7%in media conditions (FIG. 27D). Although there was a considerable proportion of plasticized CD4+CD25+ T cells in anti-CD3/28/137 stimulation and the culture media group as well (FIG. 27C), the plasticized (CD4+CD25+) T cells in these groups proliferated less, resulting in significantly higher proliferation index of CD4+CD25− T cells (54.4% in anti-CD3/28/137 stimulation P<0.007, 48.1% in culture media group P<0.06) (FIG. 27D). It is believed that the intermediate Treg cell population (CD4+CD25−IFNg+IL17+FOXP3+) is either a functionally altered T regulatory cell population that has acquired an effector phenotype (gain of IFNg+IL17+ transcription factors) or an activated Teff population with a transient FOXP3 expression. Therefore, these CD4+CD25−IFNg+IL17+FOXP3+ T cells proliferated differentially and significantly under antigenic stimulation in the GAD65+GC7+anti-DLL4 treated group since the cells are activated in an antigen-specific manner and undergo reprogramming towards Treg phenotype (FIG. 27E). In contrast, the blunt activation under anti-CD3/28/137 stimulation resulted in a generalized T cell activation wherein intermediate cells expressed the CD25+ phenotype after strong activation of CD4+ T cells (i.e., physiological feedback expression of CD25+ receptor to inhibit over-activation of CD4+ T cells), as observable in FIGS. 27F, 27G, wherein in the anti-CD3/28/137 stimulation and media group, only two cell populations are noticeable viz. CD25+FOXP3+ and CD25−FOXP3+ T cells. Therefore, the evolution of CD4+CD25+ T cells in the GC7+ anti-DLL4 treated group is different than the anti-CD3/28/137 stimulation and media group, as evident in the dot plot (FIG. 27E) where four different populations of CD4+ cells are visible in the GAD65+GC7+anti-DLL4 group {CD25− (IV, III. green color) transitioning into CD25+FOXP3+ (II. orange color) and CD25+highFOXP3+high (I. red color)}. This reprogramming was again validated in the studies in plasticizing CD4 deficient PBMCs, where CD8+ T cells first dedifferentiated and then re-differentiated into CD4+CD25+FOXP3+ T cells, as explained later in this example.

GAD65+GC7+Anti-DLL4 Treatment Plasticizes CD4 Deficient PBMCs into CD4+CD25+FOXP3+ Tregs Withstanding Proinflammatory Milieu

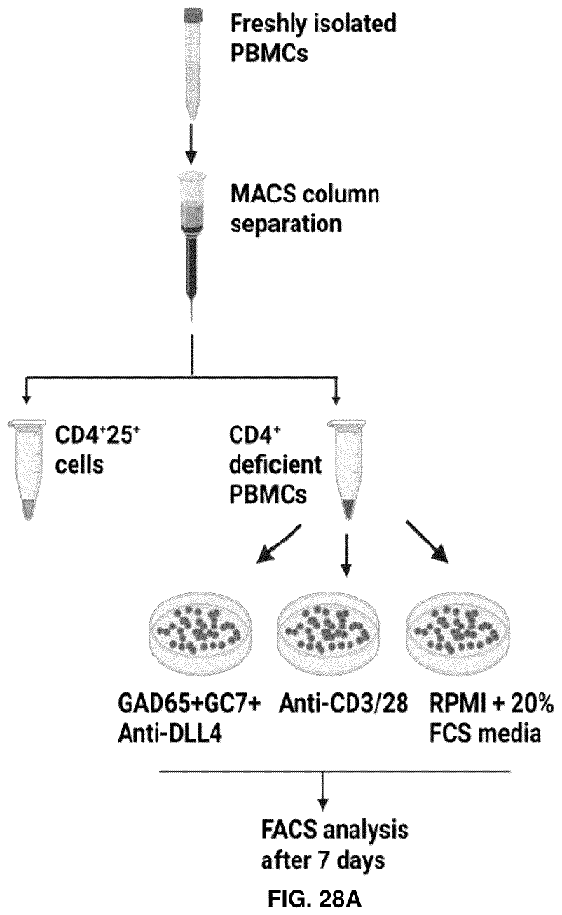

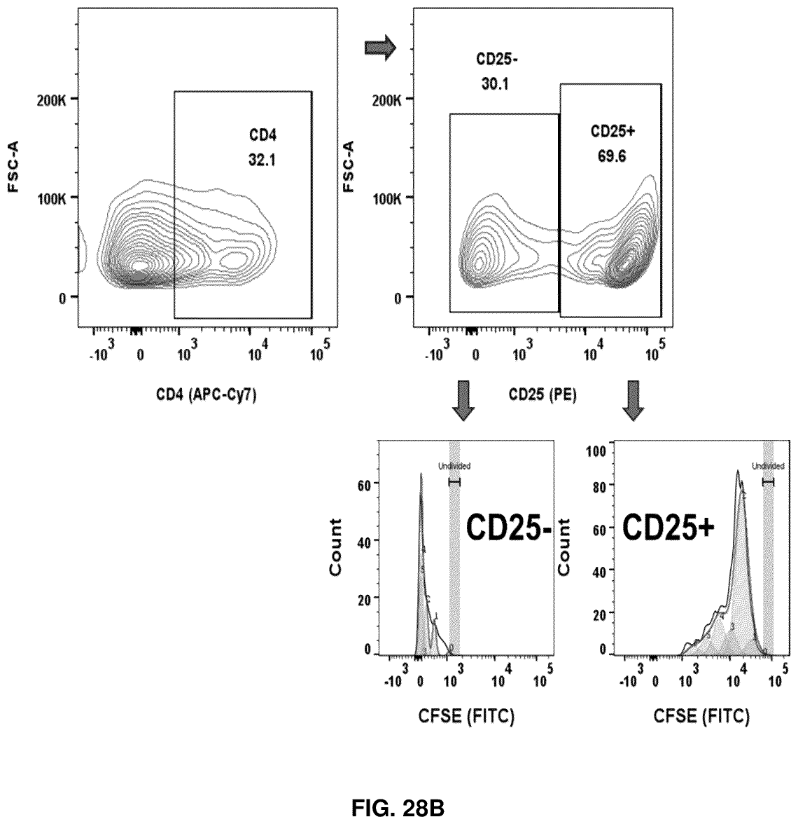

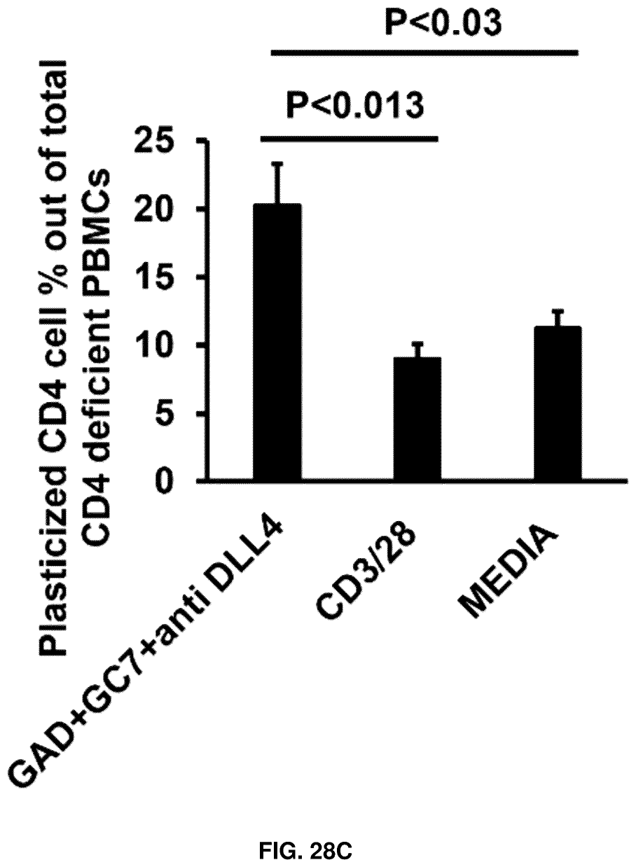

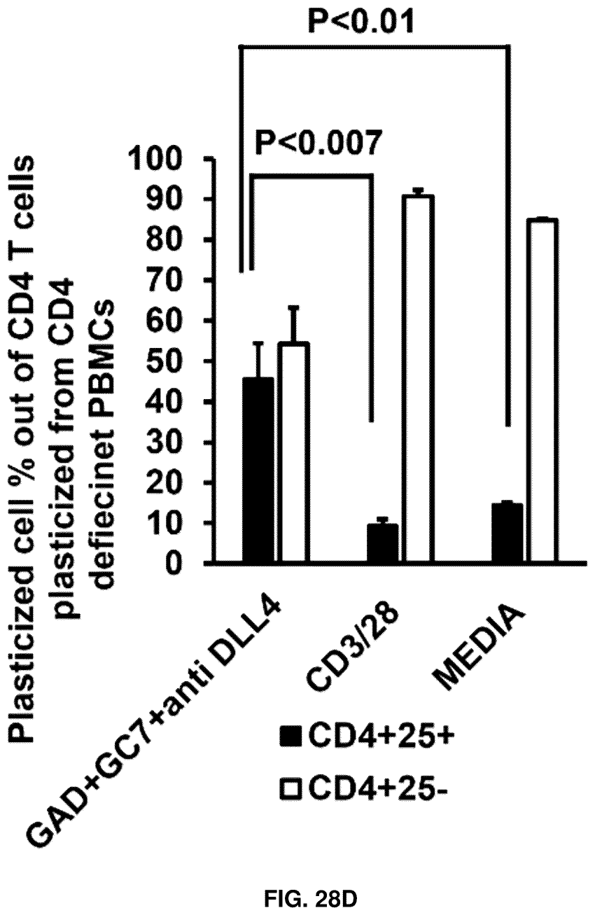

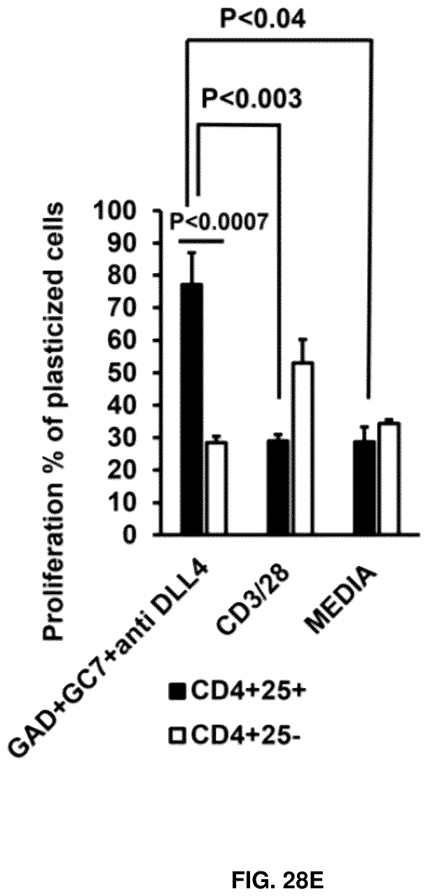

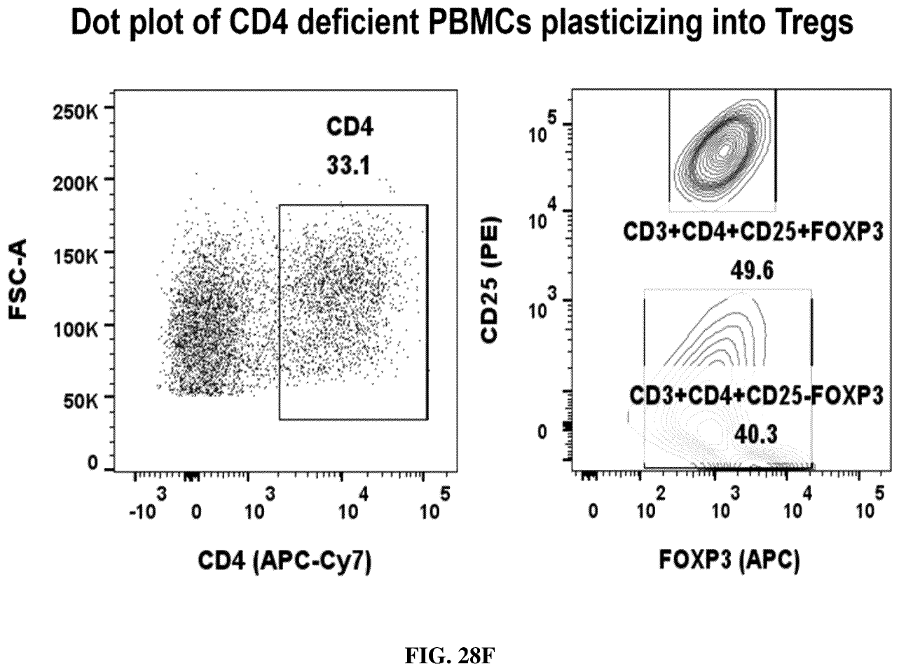

CD4+ is upregulated on CD8+ T cells with some physiological stimulations, including anti-CD3/28 co-stimulation, superantigen, and interaction with antigen-presenting cells (APCs), and these induced/activated cells can signal through the CD4 molecule, leading to preferential production of higher levels of IL-4 and expression of CD25, further indicating their functional reprogramming. The human T1D/LADA proinflammatory microenvironment was mimicked to investigate further whether combination treatment of GC7+anti-DLL4 could plasticize CD8+ T cells from CD4 deficient PBMCs into Tregs, withstand the proinflammatory conditions, and be capable of plasticizing cells independent of the cytokine milieu. CD4+ T cells were sorted from PBMCs of patients with T1D/LADA and a CD4 T cell-deficient cell population was harvested. This cell population was composed of CD8+ T cells along with APCs such as monocytes, neutrophils, dendritic cells, NK cells, etc., which are responsible for the activation of CD8+ T cells into cytotoxic T lymphocytes (CTLs, CD8+ T cells producing IFNg) (FIG. 28A). CD4 deficient PBMCs having CD8+ T cells with APCs were subjected to GAD65+GC7+anti-DLL4 treatment for 7 days wherein GAD65 specific CD8+ T cells and APCs recognized and presented GAD65 antigen to effector T cells as in T1D/LADA condition. Seven days after GAD65+GC7+anti-DLL4 treatment, the phenotype of cells was analyzed by flow cytometry, which clearly revealed that 20.3±3.1% of CD8+ T cells plasticized to CD4+ T cells (FIGS. 28B, 28C, 28F). These plasticized CD4+ T cells further underwent differentiation, and 45.6±8.8% of these CD4+ T cells expressed CD25+FOXP3+ phenotype (FIGS. 28B, 28D, 28F). As explained above, the possible mechanism is believed to be dedifferentiation of CD8+ T cells into double negative or double-positive T cells followed by redifferentiation into single positive CD4+ T cells expressing regulatory phenotype (CD25+FOXP3+). Moreover, 9.0+1.0% and 11.2+1.2% of CD4 deficient PBMCs in anti-CD3/28/137 stimulation and media group also plasticized into CD4+ T cells (FIG. 28C) which is a phenomenon under physiological stimulations. Furthermore, the newly plasticized CD4+CD25+ Tregs had a better proliferation potential than CD4+CD25+ Tregs plasticized from anti-CD3/28/137 stimulation (P<0.003) and media group (P<0.04) (FIG. 28E). This experiment clearly indicates that combination therapy of GC7+anti-DLL4 can withstand proinflammatory conditions and is capable of milieu-independent plasticization of CD4+/CD8+ T cells into functional Tregs cells (FIGS. 27E, 28E). The present data shows that the emergence of T cell plasticity is an evolving paradigm shift in clinical immune cell biology.

### Plasticized Tregs (CD4+CD25+FOXP3+) Cells Exhibited a Functional Regulatory Phenotype

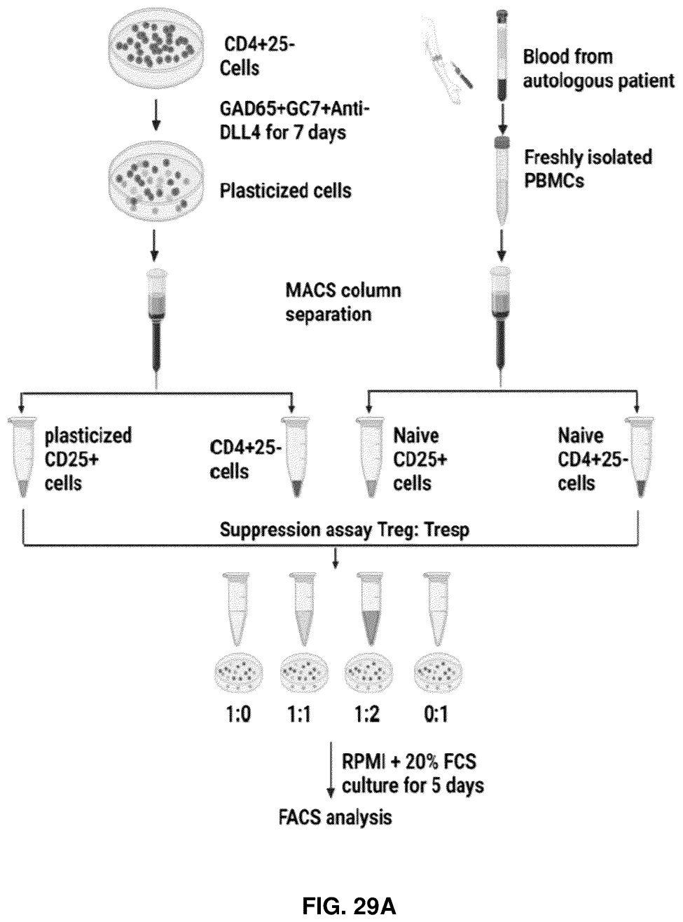

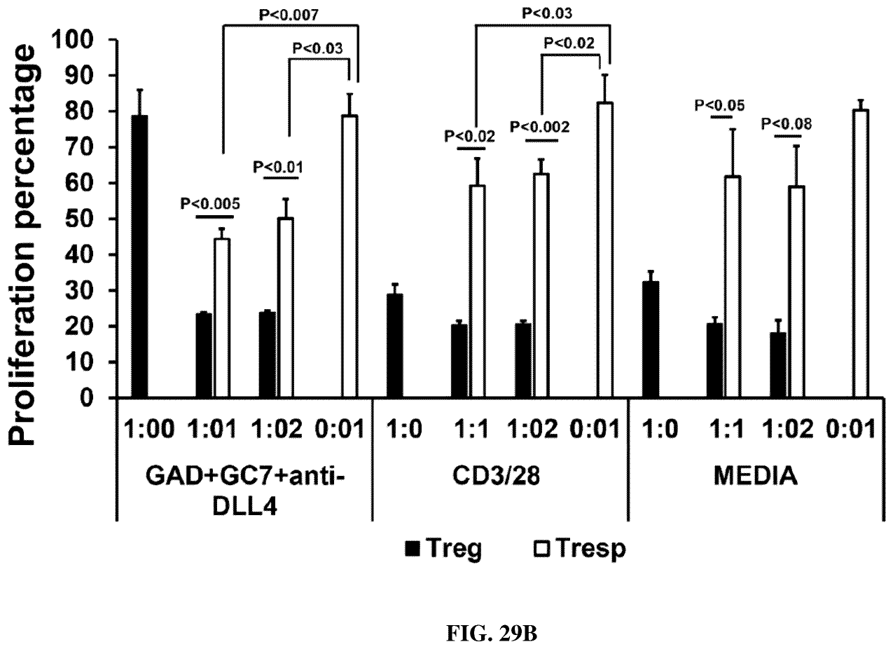

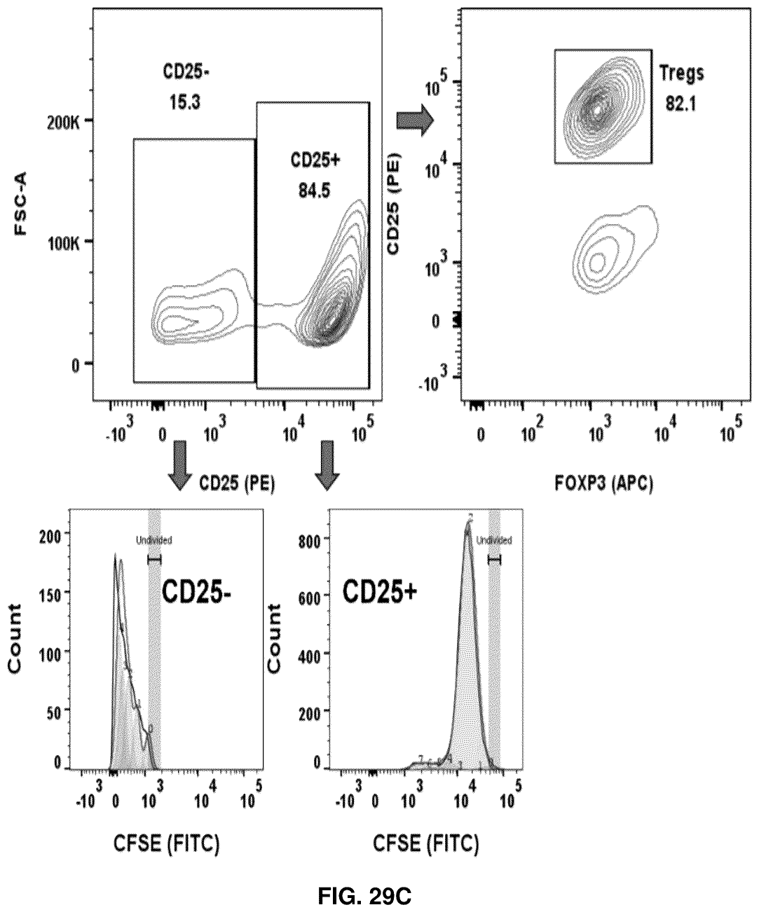

Transforming growth factor β (TGF-β) induced Tregs are unstable in sustaining in vivo suppression of Teffs, which limits their therapeutic application. Moreover, in humans, activated T cells transiently upregulate FOXP3+ without acquiring a Treg phenotype and function. Therefore, the functional regulatory phenotype in newly plasticized CD4+CD25+FOXP3+ cells was investigated from different treatment wells, and their suppressive capacity against freshly isolated autologous naïve CD4+CD25− T effector cells was evaluated. The newly plasticized CD4+CD25+ T cells were column purified, and their Treg phenotype was confirmed as CD4+CD25+FOXP3+ Tregs from different treatment wells; and autologous T effector cells (Tresp, CD4+CD25−) were isolated from freshly drawn peripheral blood (FIG. 29A). Prior to co-culture, cells were stained with CFSE, washed thoroughly, and co-cultured in different ratios of 1:0, 1:1, 1:2, and 0:1 (Treg/Tresp), respectively (FIGS. 29A, 29B). After 5 days of co-culture in RPMI+20% FCS media, cells were analyzed by flow cytometry, Treg phenotype was confirmed, and significant suppression of Teffs was noticed (FIGS. 29B, 29C). Also, the newly plasticized Tregs from GAD65+GC7+anti-DLL4 treatment demonstrated a stable CD25+FOXP3+ expression after co-culture (FIG. 29C, right quarter) and superior functional suppressive capacity against the autologous T effector cells (FIG. 29B). Indeed, antigen-specific Tregs have a superior suppression potential as compared to polyclonal cells or blunt activated Tregs. In a self-regulatory feedback loop of homeostasis, CD4+ naïve T cells produce massive amounts of IL-2 and upregulate CD25, which favors CD4+ T cell activation and proliferation. The upregulation of CD25 polarizes the microenvironment, and Tregs respond to IL-2, get activated, and suppress the Teff population. The newly plasticized CD4+CD25+ Treg cells were cultured with freshly isolated autologous Teff (CD4+CD25−) cells in culture conditions devoid of any IL-2 after thorough washing with RPMI (3 times). Therefore, the proliferation of CD25+ T cells is in response to endogenous IL-2 secreted by CD4+CD25− T cells, and the subsequent suppression of the Teff population simulates the in vivo immune regulatory response. These findings show that newly plasticized Tregs had a stable CD25+FOXP3+ phenotype and functional suppressive capacity.

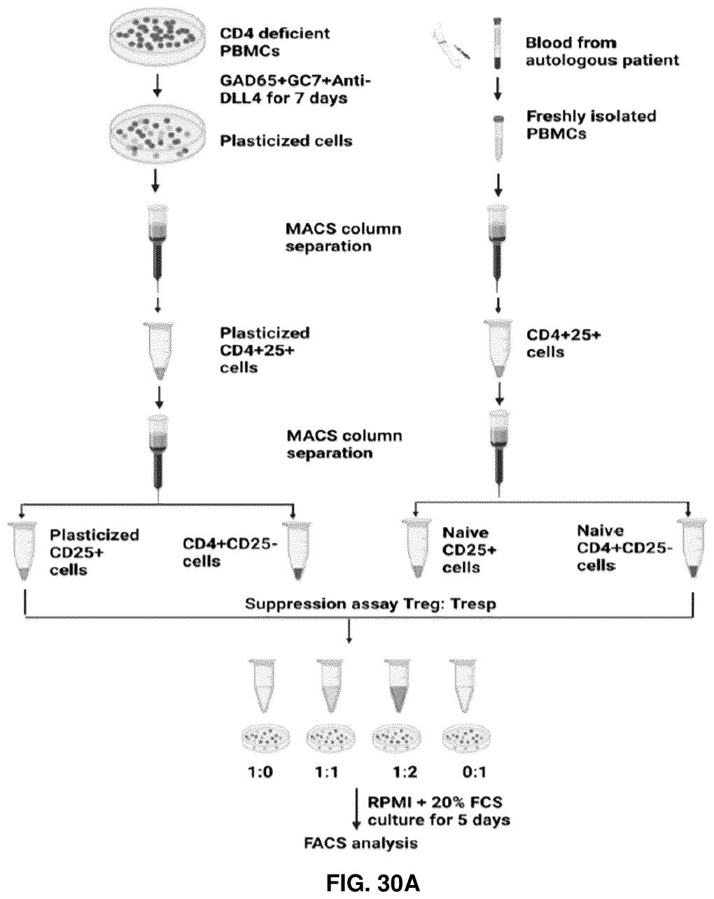

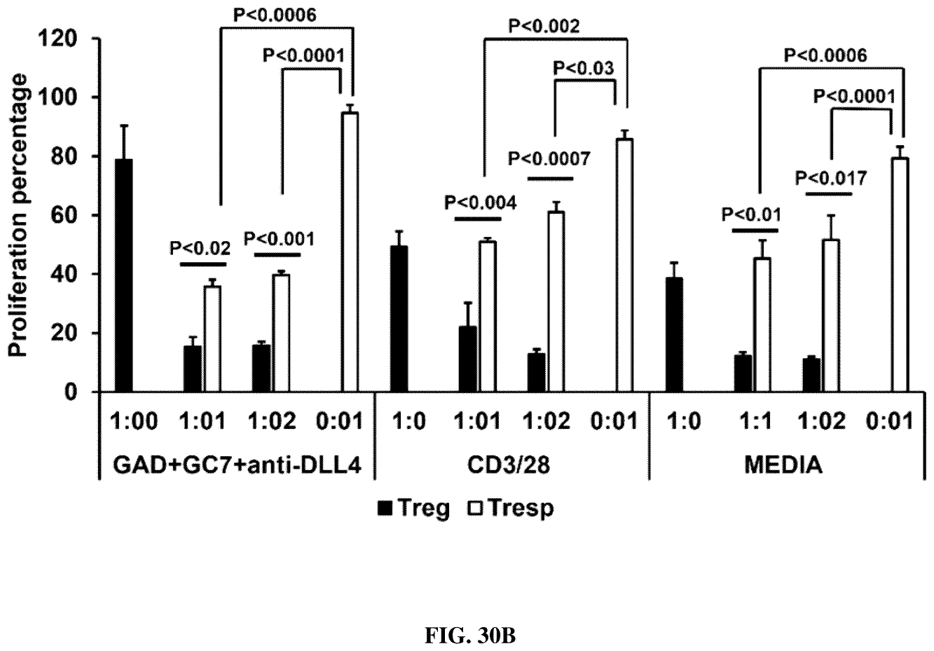

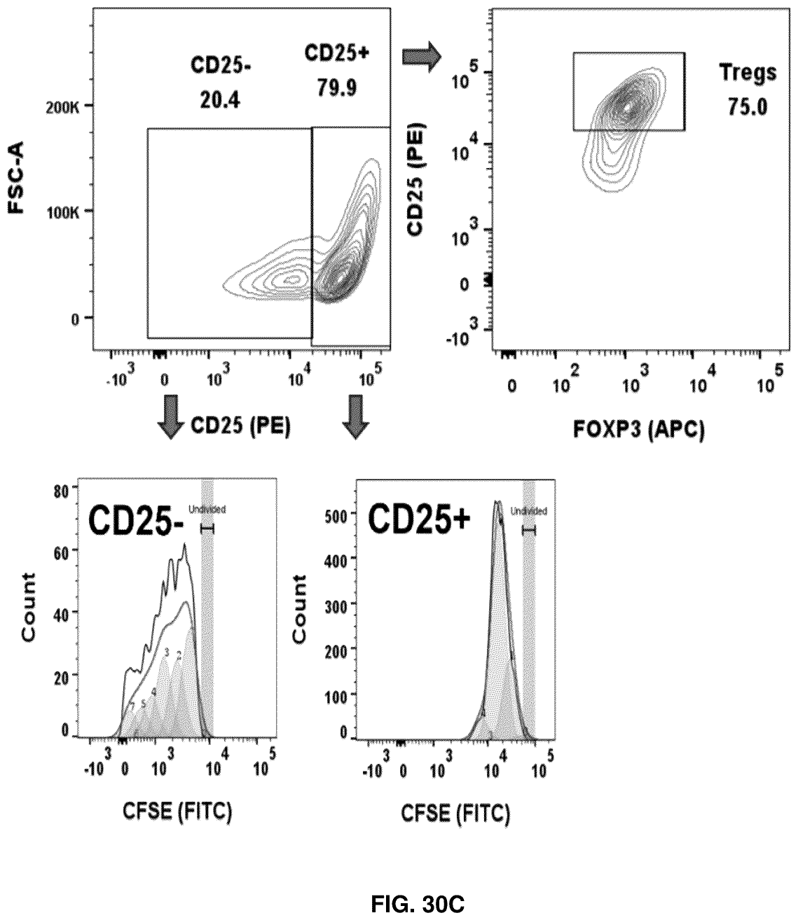

Similarly, the newly plasticized Tregs from CD4 deficient PBMCs (CD8+ T cells along with APCs) were also column purified, confirmed for Treg phenotype (CD4+CD25+FOXP3+), and co-cultured with freshly isolated autologous T effector cells (Tresp, CD4+CD25−) in different ratios of Treg/Tresp 1:0, 1:1, 1:2, and 0:1, respectively (FIG. 30A). Before co-culture, cells were incubated with CFSE for 20 mins and properly washed (3 times). The Tregs that plasticized from GAD65+GC7+anti-DLL4 group were significantly suppressing the T effector population. (FIG. 30B). Of note, the Tregs that were plasticized from the conventional anti-CD3/28/137 stimulation group and control (culture media) group also exhibited significant suppression, which per se is the physiological role of Tregs. However, in pathological conditions, there is derangement of Treg: Tresp ratios; therefore, in that scenario, the Tregs are unable to effectively suppress the Teff population such as in T1D/LADA. Since the ratio of Tresp: Tregs are the same in all three groups, the Tregs that plasticized from anti-CD3/28/137 stimulation group and culture media group also exhibited significant suppression of Tresp cells (FIG. 30B).

Immunomodulatory GC7+anti-DLL4 Treatment did not have any Adverse Effects on the Immune Cells

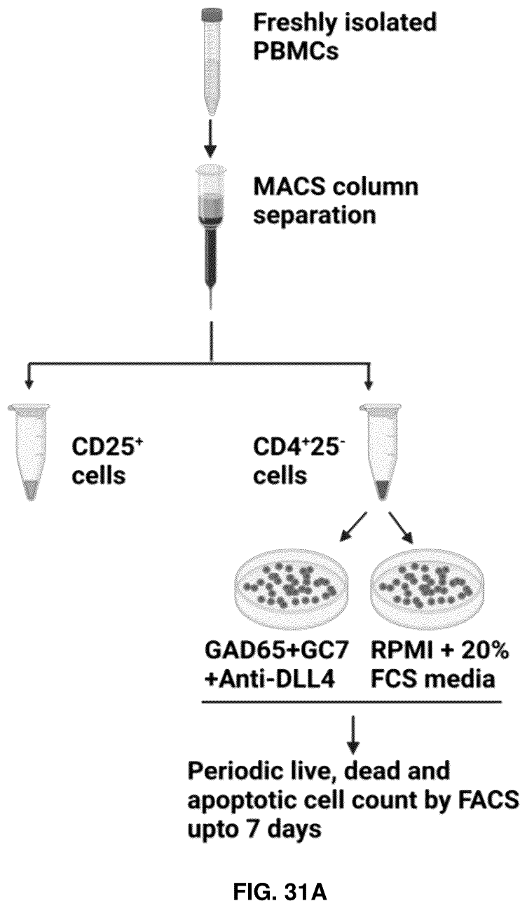

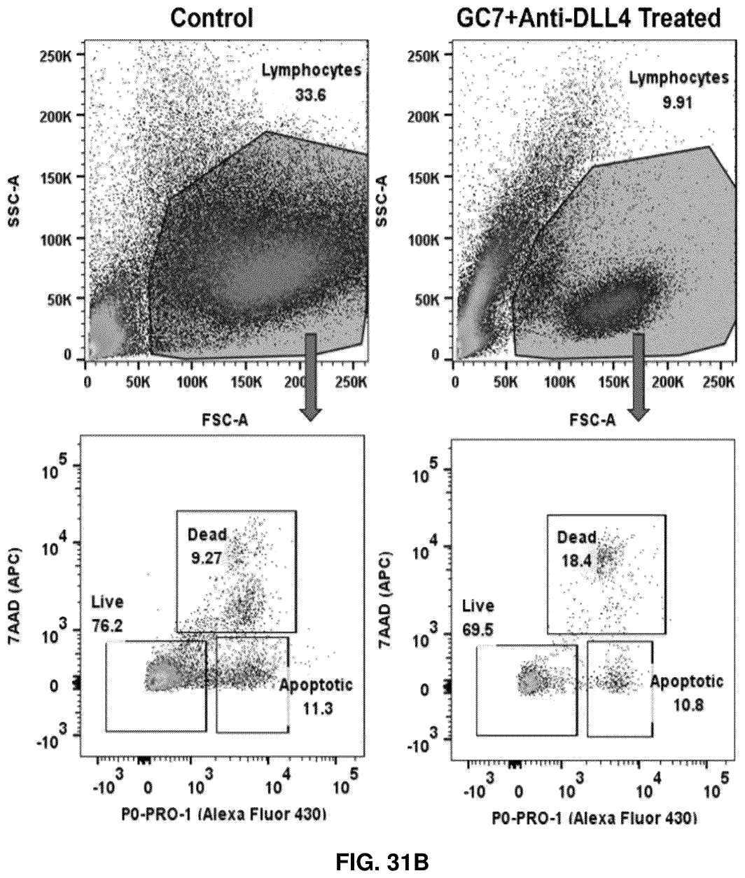

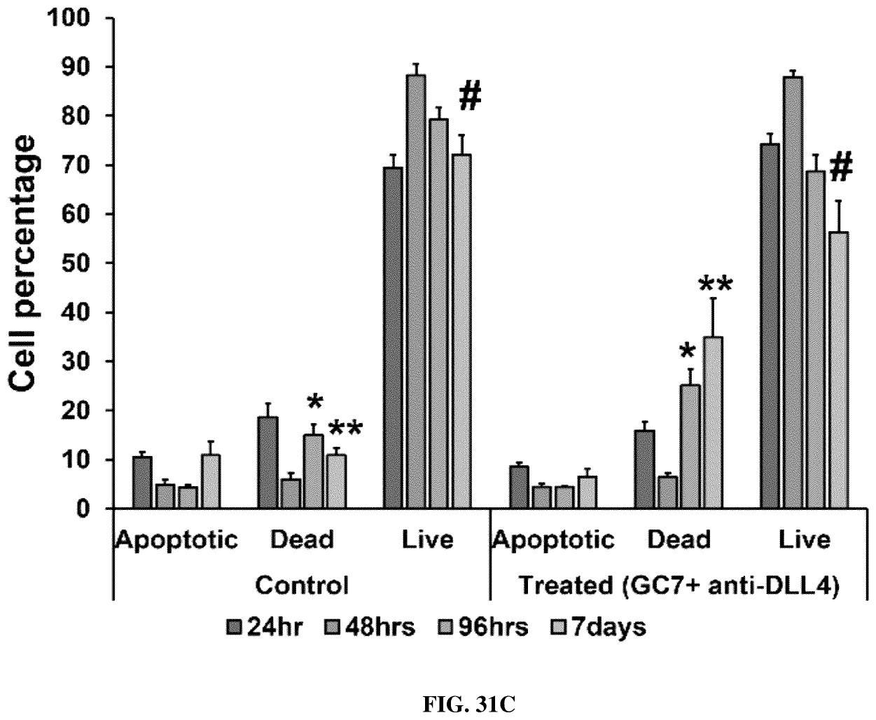

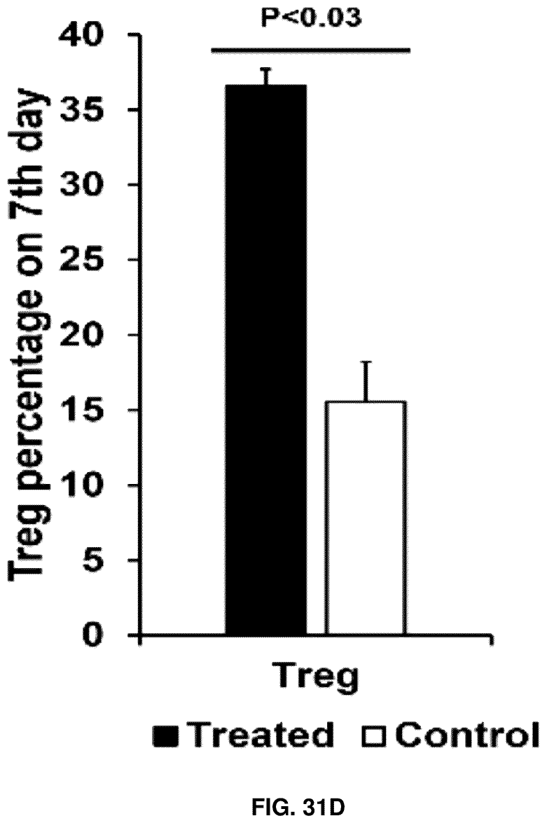

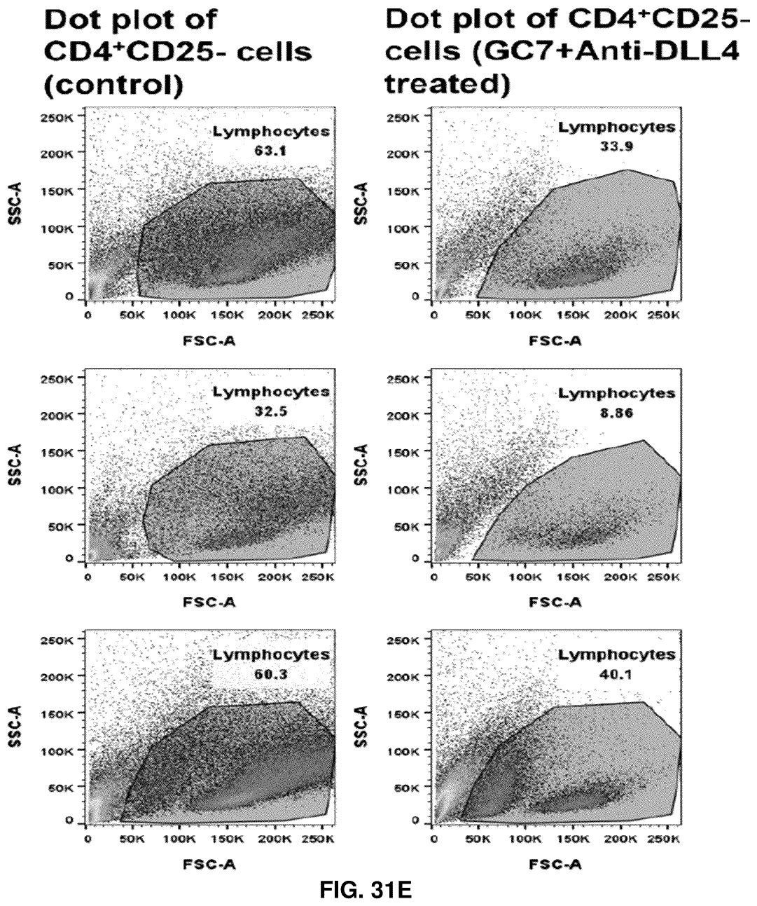

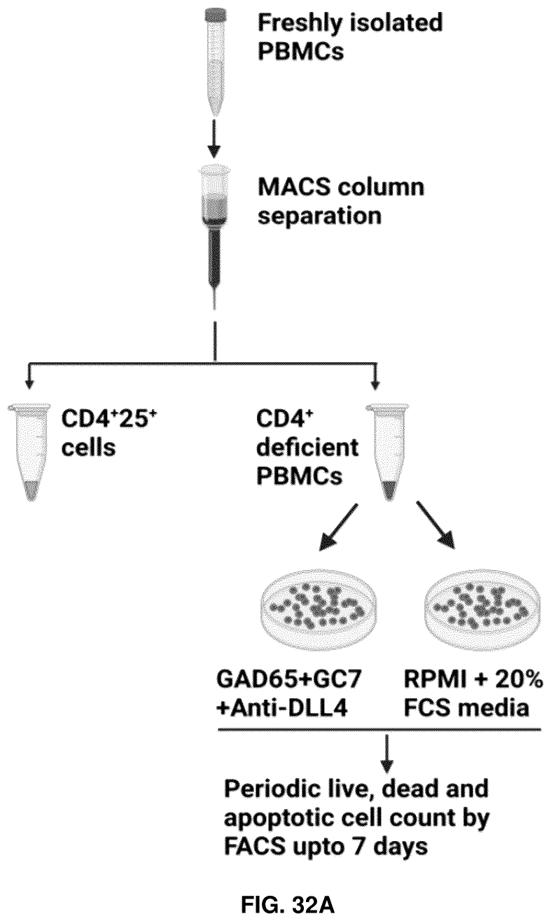

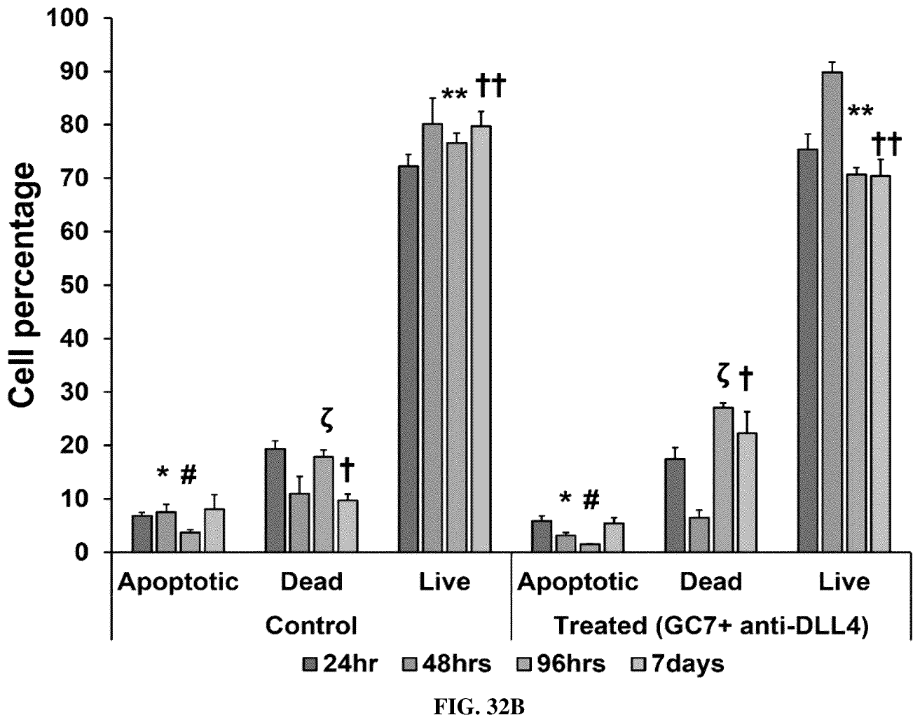

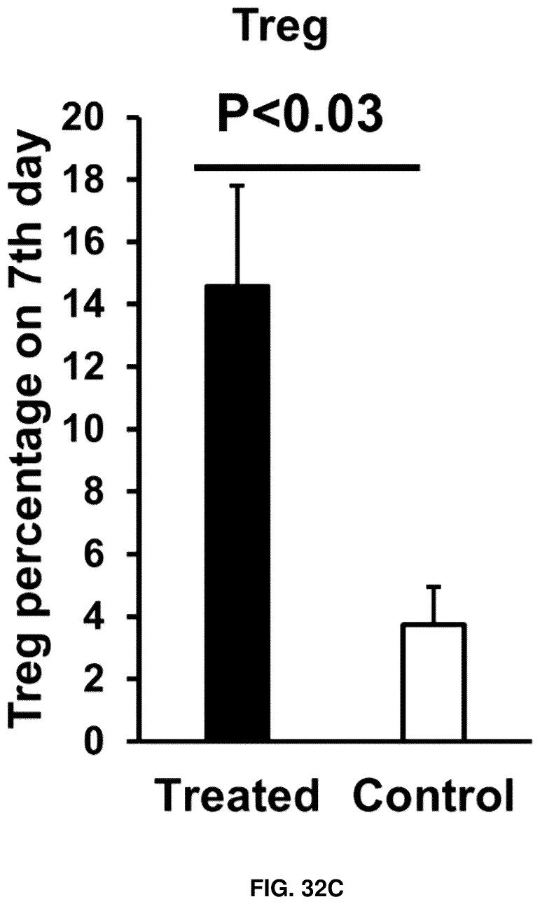

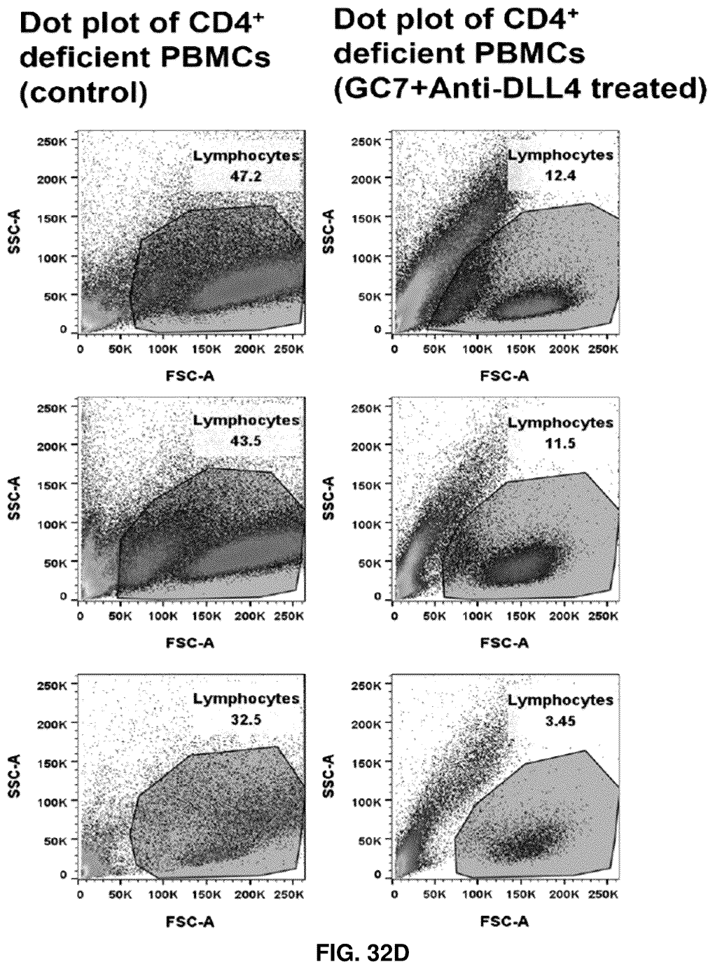

Previous in vivo studies on T1D mice and NOD mice did not reveal any significant adverse effect of using GC7 or anti-DLL4. However, to validate the results on human immune cells, a longitudinal time-based assessment of cell viability and apoptosis was performed in purified CD4+CD25− T cells and CD4 deficient PBMCs undergoing immunomodulation with GAD65+GC7+anti-DLL4 for 7 days (FIGS. 31A-31B, 32A). In Treg deficient CD4+ T cells, no difference in the live, dead, and apoptotic cell populations was observed in treated and control groups until 48 hours of treatment (FIG. 31C). Post 96 hours, the dead cell count was significantly increased (P=0.003) in the GAD65+GC7+anti-DLL4 treated group, wherein 25.1+3.4% of T cells were dead compared to 14.9+2.2% in the control group (FIG. 31C). Similarly, the live T cell percentage decreased significantly (P=0.004) in the treated group (68.7±3.3%) compared to 79.2±2.6% in the control group (FIG. 31C). To investigate the reason for the spike in dead cells in the treated group, the phenotype of cells in both treated and control groups was analyzed on the 7^{th} day. The flow cytometry analysis revealed that 36.6% of live T cells in the treated group (FIGS. 31D-31E) were newly plasticized Tregs due to AD65+GC7+anti-DLL4 treatment expressing their suppressive phenotype and killing the Teff population (FIG. 31E). Hence, the suppression of Teffs was reflected as an increase in dead cell count. This was confirmed by the cell phenotype of live cells in the control group of autologous patient's cells, in which most of the cells were non-regulatory (FIG. 31D) and hence, the comparatively fewer cells observed dead were the ones undergoing physiological cell death. In the CD4 deficient PBMCs, while there were no changes observed in live, dead, and apoptotic cells for the first 24 hours in GAD65+GC7+anti-DLL4 treated and control groups (FIG. 32B), there was a significant increase in apoptotic cells in the control group (7.5±1.5%) as compared to the treated group (3.1±0.5%) at 48 hours (P=0.021) (FIG. 32B). This indicates that the GC7+anti-DLL4 treatment may have apoptosis-delaying effects on the immune cells. Further, like the trend observed during the plasticity of purified CD4+CD25− T cells post 96 hours, there was a significant sudden spike in dead cell count in the CD4 deficient PBMCs in the GAD65+GC7+anti-DLL4 treated group post 96 hours (P=0.00015) which continued until day 7 with a significantly increased dead cell count in the treated group (P=0.0132) (FIG. 32B). Here it was again observed that the phenotype of live cells on the 7^{th} day was T regulatory (14.6+3.2%) in the treated group compared to 3.7±1.2% in the control group (FIGS. 32C-32D). This indicates that plasticized Tregs are exhibiting their functional suppressive phenotype, suppressing the Teff population, which is manifested as an increase in dead cell count in the treated group beyond 96 hours (FIGS. 32B-32D) and not any adverse effect of immunomodulatory treatment on the viability of immune cells. Hence, the use of GAD65+GC7+anti-DLL4 did not exhibit cellular toxicity in the concentration range that plasticizes CD4+ T cells or CD4 deficient PBMCs into Tregs.

### Discussion

For the treatment of autoimmune diseases, attempts for in vivo induction of Tregs with low-dose IL-2 therapy or TGF-β, have encountered significant limitations for use in clinical practice, because of the pleiotropic role of these cytokines and activation of cells other than Tregs (cosinophils, NK cells, CD8+ T cells). In the case of TGF-β, a lower concentration, particularly in the presence of inflammatory cytokines such as IL-6 and IL-21, may preferentially promote Th17 response and hence exacerbate Treg/Th17 imbalance. Similarly, TNF blockers may paradoxically induce Th1/Th17 cells and dysregulate IFN response. In T1D/LADA, therapeutic interventions have focused either on symptomatic treatment, mitigating the beta cell stress or polyclonal expansion of the Tregs; however, truly advantageous combination therapy should have a multipronged approach enriching Tregs, restraining CTLs, and decreasing the ER stress in the beta cells simultaneously.

The immunophenotype of T1D constitutes a reduced number of Treg cells, too low in number to stop perpetuating immune assault or unfit to confine the population of T effectors attacking pancreatic beta cells. Also, aberrant plasticity of Treg cells is observed, with Treg cells expressing pro-inflammatory cytokines, acquiring T helper-like phenotypes, and displaying diminished function in most cases but maintaining FOXP3+ expression levels. The intermediate population of Treg cells maintains the FOXP3+ expression but also expresses proinflammatory cytokines in patients with T1D/LADA (FIGS. 26B-26C). There is an increased frequency of IFNg+FOXP3+ Treg cells in the periphery of patients with autoimmune diseases such as T1D, multiple sclerosis, autoimmune hepatitis, and Sjögren syndrome, that display reduced suppressive capacities as compared to Treg cells from healthy age-matched subjects. This pathological conversion of Tregs into Teff type or Th17 type is instrumental to visualize CD4+IFNg+IL17+FOXP3+ cells significantly increased in patients with recent-onset T1D/LADA (FIG. 26B). These CD4+IFNg+IL17+FOXP3+ intermediate cells are CD25− T cells (FIG. 26C) like in EAE wherein approximately 40% of cells infiltrating the central nervous system are CD4+FOXP3+CD25− T cells. These transient Tregs play a role in promoting inflammation, as in multiple sclerosis, where FOXP3+IFNg+ Tregs are significantly abundant. A similar observation has been made in animal models and patients with inflammatory bowel disease (IBD), wherein Tregs expressing IFNg and IL-17 enhance inflammation. This significantly expanded population of transient Tregs has been previously characterized as unfit Tregs but, with therapeutic intervention (as demonstrated in this example), can be reverted to functionally active Tregs (FIGS. 27E, 28E, 31D, 32C).

This highly plastic intermediate subset originates from CD4+CD25− T cells on encountering an antigen, receiving extrathymic signals, or proinflammatory milieu. Tregs are converted into Teffs or IL17+IFNg+FOXP3+ T cells with subsequent loss of FOXP3+ expression in chronic inflammation conditions. Thus, the inflammatory microenvironment is responsible for tipping these intermediate subsets towards inflammatory phenotype. Contemporary TNFα blockers augment the phosphorylation of FOXP3+ and modulate aberrant trans-differentiation of Tregs in rheumatoid arthritis. In the present example, CD4+CD25− T cells of patients with T1D/LADA showed an enriched population of CD4+CD25−IFNg+IL17+FOXP3+ T intermediate cells, which on treatment with GC7 and anti-DLL4 transdifferentiated into CD4+CD25+FOXP3+ Treg cells (FIGS. 27C, 27E).

Furthermore, plasticization of Tregs from a pool of PBMCs entirely devoid of CD4+ T cell population is shown in this example (FIGS. 28A, 28C, 28D, 28F). This CD4 deficient pool of PBMCs mimics the proinflammatory cytokine milieu typical of T1D/LADA wherein APCs activate GAD65-specific CD8+ T cells. This illustrates combination therapy of GAD65+GC7+anti-DLL4 can induce differentiation of CD8+ T cells into CD4+ T cells and subsequently plasticization into CD4+CD25+FOXP3+ Tregs in proinflammatory conditions (FIG. 28F). It is noteworthy since pro-inflammatory conditions hamper the induction of Tregs in vivo conditions and hence limit the therapeutic potential of previously used immunomodulators such as TGF-β. TGF-β induced FOXP3+ expression of Tregs is hampered in culture conditions containing inflammatory cytokines such as IL-12, IL-4, and IL-6. Notably, in the present example, the induction and expansion of CD25+FOXP3+ T cells was achieved without the use of IL-2 or TGF-β. Supported by the results in the previous examples, from in vivo studies on GC7 and Notch inhibition where the intrathymic differentiation and enrichment of Tregs was elucidated, these in vitro results address the thymic-independent role of adoption of Treg cell fate by CD4+and CD8+ T cells. Although there are reports of plasticity of Tregs and Th17 cells, the existence of CD4+CD25−IFNg+IL17+FOXP3+ T cells in patients with T1D/LADA is not known, and differentiation of these cells into functional Tregs from Treg deficient CD4+ T cells and CD4 deficient PBMCs can be harnessed for therapeutic interventions in autoimmune diseases.

Since inhibition of the polyamine pathway alters overall chromatin accessibility and rewires Th17 specific towards Treg-specific transcriptome and chromatin state, this may be the mechanism of rewiring this intermediate Treg subset into stable Tregs. On the other hand, the effects of Notch inhibition enhancing peripheral Tregs independent of thymus may be attributed to mTOR pathway inhibition and increased STAT5 phosphorylation. In vitro, induction of FOXP3 by pharmacological inhibition of the CDK19 gene leading to enhanced activation of STAT5 also emphasizes the role of the IL-2/STAT5 pathway in acquiring the Treg phenotype. However, considering the web of signaling networks and its complexities, it is hard to decipher the actual changeover of signals in real time. Nevertheless, the newly plasticized Tregs displayed a functional phenotype and significantly suppressed the autologous T effector cells, which indicates a stable expression of CD25+FOXP3+ suppressive phenotype (FIGS. 29B, 29C, 30B, 30C). Flow cytometry analysis revealed they maintained their suppressive phenotype postsuppressive assay, negating interchangeability into T effector Tregs (FIG. 29C, right upper quarter; FIG. 30C, right upper quarter). Moreover, plasticized Tregs from GAD65+GC7+anti-DLL4 had better proliferative potential than conventional CD3/CD28/137 stimulation (FIGS. 27D, 28D).

Hyp-eIF5a is over-expressed in CD4+ activated T cells in T1D and exacerbates diabetic phenotype. GC7 inhibits hyp-eIF5a without changing the basal levels, hence having a restrictive role in the translation of a specific set of proteins. Moreover, it is believed that there is depletion of eIF5a impaired translational elongation of only about 5% mRNA; therefore, depletion of eIF5a with GC7 may not produce any generalized adverse effects. The results in the present example show there were no adverse effects of using GC7 and anti-DLL4 on CD4+ T cells and CD4 deficient PBMCs (FIGS. 31-32). Autophagy in a cancer cell line can be induced within 24 hours of treatment with GC7. However, the observation in the present example of immune cells did not reveal any changes in apoptotic cell percentage and live/dead cells for up to 48 hours, indicating that GC7 does not universally induce autophagy (FIGS. 31C, 32B). The transient nature of autophagy further supports this since it is a short-lived cellular adaptation of cells to stress, evident within hours, and cannot be sustained for long. The phenotype of cells on the 7^{th} day clearly revealed that the Tregs exerted their suppressive phenotype on Teffs cells leading to cell death and an increase in dead cell count post 96 hrs. Further, compounds such as Semapimod which inhibit deoxyhypusine synthase (DHS) with an IC₅₀ around 2 mM, used in clinical trials in humans for Crohn's disease and Endoscopic Retrograde Cholangiopancreatography, were well tolerated by patients; therefore, GC7 displaying an IC₅₀ of 17 nM should not have any significant adverse effects. The experiments in the present example, which revealed no adverse effects on the cell viability with immunomodulatory treatment. and alongside previous studies in the humanized mice model of T1D and DHPS+/− mice which did not lead to any significant suppression of cellular proliferation and maintained normal growth, demonstrate a reassuring safety profile and strategic use of this treatment strategy. Antigen-specific Treg enrichment and depletion of CTLs is the holy grail of LADA/T1D cure. The findings in this example represent the culmination and ultimate synthesis of the preceding findings on the use of GC7 in human and mice islets and spontaneous humanized T1D mice, anti-DLL4 inhibition in NOD mice, and spontaneous humanized T1D mice. The addition of findings from human in vitro studies serves as a definitive endpoint in exploring immune resetting using simultaneous inhibition of Notch and eIF5a in LADA/T1D.

### CONCLUSION

This example identified a new population of CD4+CD25−IFNg+IL17+FOXP3+ T cells in T1D/LADA patients that successfully reverted to functionally active Treg cells suppressing diabetogenic T effector cells. Without wishing to be bound by theory, it is believed that in the GAD65+GC7+anti-DLL4 treated group, CD8+ first dedifferentiate into double-negative/double-positive T cells and then redifferentiate into specific CD4+CD25+ T cells expressing FOXP3. Most importantly, this approach can plasticize Tregs in a proinflammatory microenvironment without adversely affecting the immune cells. Thus, this immunomodulatory treatment with GC7 and anti-DLL4 addresses the immunological cascade of events in T1D as well as metabolic/pathophysiological inflammation-mediated ER stress, and facilitates the design of immunotherapy for LADA/T1D and other autoimmune diseases.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A method for inducing plasticity in intermediate Treg cells, the method comprising:
contacting intermediate Treg cells with an effective amount of an eIF5A inhibitor and an effective amount of a Notch signaling inhibitor so as to induce plasticity in the intermediate Treg cells to exhibit a regulatory T cell phenotype.

2. The method of claim 1, wherein the eIF5A inhibitor comprises GC7.

3. The method of claim 1, wherein the Notch signaling inhibitor comprises an anti-DLL4 antibody.

4. The method of claim 1, wherein the eIF5A inhibitor comprises GC7 and the Notch signaling inhibitor comprises an anti-DLL4 antibody.

5. The method of claim 1, wherein the intermediate Treg cells are CD4+IFNg+IL17+FOXP3+ T cells.

6. The method of claim 1, wherein the regulatory T cell phenotype is CD4+CD25+FOXP3+.

7. The method of claim 1, wherein the eIF5A inhibitor and the Notch signaling inhibitor are administered simultaneously.

8. A method for inducing plasticity in intermediate Treg cells to exhibit a regulatory T cell phenotype, the method comprising:
administering an effective amount of an eIF5A inhibitor to a subject so as to inhibit eIF5A in the subject; and
administering an effective amount of a Notch signaling inhibitor to the subject so as to inhibit Notch signaling in the subject;
wherein eIF5A and Notch signaling in the subject are inhibited simultaneously so as to induce plasticity in intermediate Treg cells in the subject to exhibit a regulatory T cell phenotype.

9. The method of claim 8, wherein the eIF5A inhibitor comprises GC7.

10. The method of claim 8, wherein the Notch signaling inhibitor comprises an anti-DLL4 antibody.

11. The method of claim 8, wherein the eIF5A inhibitor and the Notch signaling inhibitor are administered simultaneously.

12. The method of claim 8, wherein the eIF5A inhibitor comprises GC7 and the Notch signaling inhibitor comprises an anti-DLL4 antibody.

13. The method of claim 8, wherein the intermediate Treg cells are CD4+IFNg+IL17+FOXP3+ T cells.

14. The method of claim 8, further comprising administering a treatment for type 1 diabetes to the subject while eIF5A and Notch signaling are inhibited in the subject.

15. The method of claim 8, wherein the eIF5A inhibitor and the Notch signaling inhibitor are administered simultaneously.

16. The method of claim 8, wherein the regulatory T cell phenotype is CD4+CD25+FOXP3+.

17. A method of diagnosing a subject with T1D or LADA, the method comprising:
obtaining a blood sample from the subject;
analyzing the blood sample to determine an amount of CD4+CD25−IFNg+IL17+FOXP3+ T cells present;
comparing the determined amount of CD4+CD25−IFNg+IL17+FOXP3+ T cells present in the blood sample from the subject to a control amount of CD4+CD25−IFNg+IL17+FOXP3+ T cells present in a control sample from a donor without T1D or LADA; and
diagnosing the subject as having T1D or LADA if the amount of CD4+CD25−IFNg+IL17+FOXP3+ T cells present in the blood sample is greater than the control amount.

18. The method of claim 17, further comprising determining the subject does not have T1D or LADA if the amount of CD4+CD25−IFNg+IL17+FOXP3+ T cells present in the blood sample is less than the control amount.

19. The method of claim 17, wherein the blood sample comprises peripheral blood from the subject.
